(19) United States
(12) Patent Application Publication
(10) Pub. No.: US 2019/0218214 A1
(43) Pub. Date: Jul. 18, 2019

(54) Inhibition of BMP Signaling Compounds, Compositions and Uses Thereof

(71) Applicant: Vanderbilt University, Nashvile, TN (US)

(72) Inventors: Corey R. Hopkins, Nolensville, TN (US); Charles C. Hong, Nolensville, TN (US); Craig W. Lindsley, Brentwood, TN (US); Darren W. Engers, Nashville, TN (US)

(21) Appl. No.: 16/333,201

(22) PCT Filed: Sep. 14, 2017
(86) PCT No.: PCT/US17/51557

§ 371 Date: Mar. 13, 2019

Related U.S. Application Data
(60) Provisional application No. 62/394,584, filed on Sep. 14, 2016.

(51) Int. Cl. C07D 471/04; C07D 519/00; A61P 35/00

(52) U.S. Cl. CPC C07D 471/04; A61P 35/00; C07D 519/00

(57) ABSTRACT

Provided herein are substituted imidazo[1,2-a]pyridines useful as inhibitors of BMP signaling. The invention further provides pharmaceutical compositions of the compounds of the invention. The invention also provides medical uses of substituted imidazo [1,2-a] pyridines.

### RELATED APPLICATION

This application claims the benefit of priority to U.S. Provisional Patent Application Ser. No. 62/394,584, filed Sep. 14, 2016, which is hereby incorporated herein by reference in its entirety.

### BACKGROUND

Signaling involving the Transforming Growth Factor β (TGF-β) superfamily of ligands is central to a wide range of cellular processes, including cell growth, differentiation, and apoptosis. TGF-β signaling involves binding of a TGF-β ligand to a type II receptor (a serine/threonine kinase), which recruits and phosphorylates a type I receptor. The type I receptor then phosphorylates a receptor-regulated SMAD (R-SMAD; e.g., SMAD1, SMAD2, SMAD3, SMAD5, SMAD8 or SMAD9), which binds to SMAD4, and the SMAD complex then enters the nucleus where it plays a role in transcriptional regulation. The TGF superfamily of ligands includes two major branches, characterized by TGF-β/activin/nodal and Bone Morphogenetic Proteins (BMPs).

Signals mediated by bone morphogenetic protein (BMP) ligands serve diverse roles throughout the life of vertebrates. During embryogenesis, the dorsoventral axis is established by BMP signaling gradients formed by the coordinated expression of ligands, receptors, co-receptors, and soluble antagonists. Excess BMP signaling causes ventralization, an expansion of ventral at the expense of dorsal structures, while diminished BMP signaling causes dorsalization, an expansion of dorsal at the expense of ventral structures. BMPs are key regulators of gastrulation, mesoderm induction, organogenesis, and endochondral bone formation, and regulate the fates of multipotent cell populations. BMP signals also play critical roles in physiology and disease, and are implicated, for example, in primary pulmonary hypertension, hereditary hemorrhagic telangiectasia syndrome, fibrodysplasia ossificans progressiva, and juvenile polyposis syndrome among others.

The BMP signaling family is a diverse subset of the TGF-β superfamily. Over twenty known BMP ligands are recognized by three distinct type II (BMPRII, ActRIIa, and ActRIIb) and at least three type I (ALK2, ALK3, and ALK6) receptors. Dimeric ligands facilitate assembly of receptor heteromers, allowing the constitutively-active type II receptor serine/threonine kinases to phosphorylate type I receptor serine/threonine kinases. Activated type I receptors phosphorylate BMP-responsive (BR-) SMAD effectors (SMADs 1, 5, and 8) to facilitate nuclear translocation in complex with SMAD4, a co-SMAD that also facilitates TGF signaling. In addition, BMP signals can activate intracellular effectors such as MAPK p38 in a SMAD-independent manner. Soluble BMP antagonists such as noggin, chordin, gremlin, and follistatin limit BMP signaling by ligand sequestration.

A role for BMP signals in regulating expression of hepcidin, a peptide hormone and central regulator of systemic iron balance, has also been suggested. Hepcidin binds and promotes degradation of ferroportin, the sole iron exporter in vertebrates. Loss of ferroportin activity prevents mobilization of iron to the bloodstream from intracellular stores in enterocytes, macrophages, and hepatocytes. The link between BMP signaling and iron metabolism represents a potential target for therapeutics.

Given the tremendous structural diversity of the BMP and TGF-β superfamily at the level of ligands (>25 distinct ligands at present) and receptors (three type I and three type II receptors that recognize BMPs), and the heterotetrameric manner of receptor binding, traditional approaches for inhibiting BMP signals via soluble receptors, endogenous inhibitors, or neutralizing antibodies are not practical or effective. Endogenous inhibitors such as noggin and follistatin have limited specificity for ligand subclasses. Single receptors have limited affinity for ligand, whereas ligand heterotetramers exhibit rather precise specificity for particular ligands. Neutralizing antibodies are specific for particular ligands or receptors and are also limited by the structural diversity of this signaling system.

Thus, there is a continuing need for pharmacologic agents that antagonize BMP signaling pathways and that can be used to manipulate these pathways in therapeutic or experimental applications.

### SUMMARY OF INVENTION

In one aspect, the invention relates to compounds having the structure of Formula I or a pharmaceutically acceptable salt thereof:

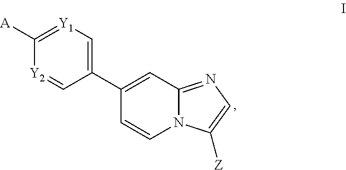

wherein A, Y¹, Y² and Z are defined herein.

In another aspect, the invention relates to pharmaceutical compositions of a compound of Formula I and a pharmaceutically acceptable carrier.

The invention also relates to methods of treating or preventing a disease or condition comprising administering a compound or composition of the invention. In certain embodiments, the disease is cancer. The invention further relates to methods of inhibiting proliferation of a cancer cell, comprising contacting a cancer cell with a compound or composition of the invention.

The invention also relates to methods of modulating the BMP signaling pathway, comprising contacting a cell with a compound or composition of the invention.

The invention also provides methods for propagating, engrafting, or differentiating a progenitor cell, comprising contacting the cell with a compound or composition of the invention in an amount effective to propagate, engraft, or differentiate the progenitor cell.

### BRIEF DESCRIPTION OF THE FIGURES

FIGS. 1A-1D depicts extracted ion chromatograms. FIG. 1A shows the chromatogram of Compound 17 with rat liver microsomes and NADPH. FIG. 1B shows the chromatogram of Compound 17 with rat liver microsomes without NADPH. FIG. 1C shows the chromatogram of Compound 17 with human liver microsomes with NADPH. FIG. 1D shows the chromatogram of Compound 17 with human liver microsomes without NADPH. Ions monitored include: 456.2+470.18+472.2+488.19+504.18+486.17+454.18+452.17+474.21+490.2+454.2+438.21+470.2+452.19+468.18+430.18+446.18+462.17+444.16+431.17+447.16+445.15+463.16+387.14+403.14+419.13+372.13+388.13+404.12+420.22+436.21+452.21+368.2+450.19

FIGS. 2A-2D depicts extracted ultraviolet chromatograms. FIG. 2A shows the chromatogram of Compound 17 with rat liver microsomes and NADPH. FIG. 2B shows the chromatogram of Compound 17 with is rat liver microsomes without NADPH. FIG. 2C B shows the chromatogram of Compound 17 with human liver microsomes with NADPH. FIG. 2D shows the chromatogram of Compound 17 with human liver microsomes without NADPH. The wavelength monitored is 362 nm.

FIG. 3 shows the proposed metabolite structures of Compound 17.

FIG. 4 depicts the extracted ion chromatogram of compound 17 neat standard at a 5 μM. Ions monitored include 456.2+430.18+458.18+387.14.

FIG. 5 is a bar graph showing the effects of Compound 17 on profiled ion channels. The dashed horizontal line associated with each bar indicates the mean effect of vehicle control wells (0.3% DMSO). Compound 17 was assayed at 10 μM.

FIG. 6 is a bar graph showing the inhibition of HAMP/HPRT1 expression by compound 17 alone and in the presence of BMP-6 (10 ng/ml). ****p<0.0001; ***p<0.001; ** p<0.01; *P<0.05; $$$$p<0.0001; $$$ p<0.001; $$ p<0.01; $P<0.05;

FIG. 7 is a bar graph showing the inhibition of HAMP/HPRT1 expression by compound 20 alone and in the presence of BMP-6 (10 ng/ml). ****p<0.0001; ***p<0.001; ** p<0.01; *P<0.05; $$$$p<0.0001; $$$ p<0.001; $$ p<0.01; $P<0.05;

FIG. 8 is a bar graph showing the inhibition of HAMP/HPRT1 expression by compound 25 alone and in the presence of BMP-6 (10 ng/ml). ****p<0.0001; ***p<0.001; ** p<0.01; *P<0.05; $$$$p<0.0001; $$$ p<0.001; $$ p<0.01; $P<0.05;

FIG. 9 is a bar graph showing the inhibition of HAMP/HPRT1 expression by compound 48 alone and in the presence of BMP-6 (10 ng/ml). ****p<0.0001; ***p<0.001; ** p<0.01; *P<0.05; $$$$p<0.0001; $$$ p<0.001; $$ p<0.01; $P<0.05;

FIGS. 10A-10D are bar graphs showing the expression (mRNA) of inflammatory markers (at 2 h, 6 h, 15 h, and 24 h) in the liver in the presence of compounds 17, 20, and 48. FIG. 10A depicts the results with Crp mRNA. FIG. 10B depicts the results with IL6 mRNA. FIG. 10C depicts the results with Saa3 mRNA. FIG. 10D depicts the results with Socs3 mRNA.

FIGS. 11A and 11B are bar graphs showing mRNA expression in the liver at 2 h, 6 h, 15 h, and 24 h in the presence of compounds 17, 20, and 48. FIG. 11A depicts the results with Hamp mRNA. FIG. 11B depicts the results with Id1 mRNA.

FIGS. 12A-12C are images of Western blots of a BMP-Smad signaling assay with compounds 17, 20, and 48 at 2 h, 6 h, 15 h, and 24 h. FIG. 12A depicts the results with compound 17. FIG. 12B depicts the results with compound 20. FIG. 12C depicts the results with compound 48.

FIG. 13 is a bar graph showing HAMP mRNA expression levels in the liver of Tmprss6−/− mice injected with compounds 17, 20, 48 and the sacrificed 2 hours after injection.

FIGS. 14A and 14B are bar graphs showing improvements in iron deficiency Tmprss6−/− mice after 4 weeks of treatment with compound 20.

FIGS. 15A and 15B are bar graphs showing serum iron levels (FIG. 15A) and transferrin saturation in Tmprss6−/− mice injected with compound 20 twice a day for seven weeks compared to wild type mice and Tmprss6−/− mice injected with mock solution.

FIGS. 16A-16D are bar graphs showing improvement in hemoglobin (FIG. 16A), in hematocrit (FIG. 16B), mean corpuscular volume (FIG. 16C), and mean corpuscular hemoglobin (FIG. 16D) in Tmprss6−/− mice treated with compound 20 for 7 weeks as compared to wild type mice and treatment with a mock solution.

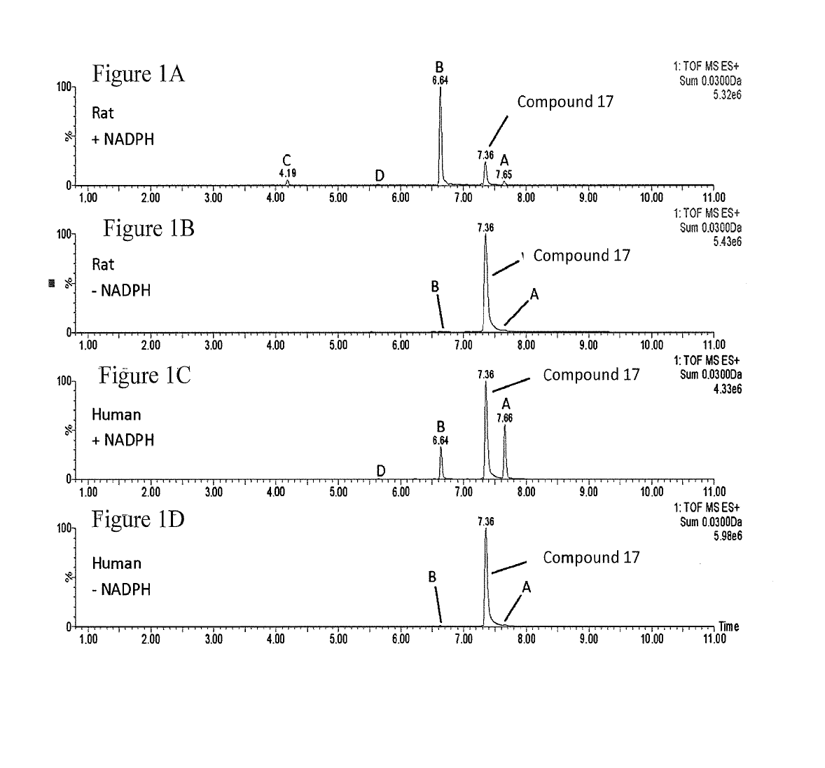

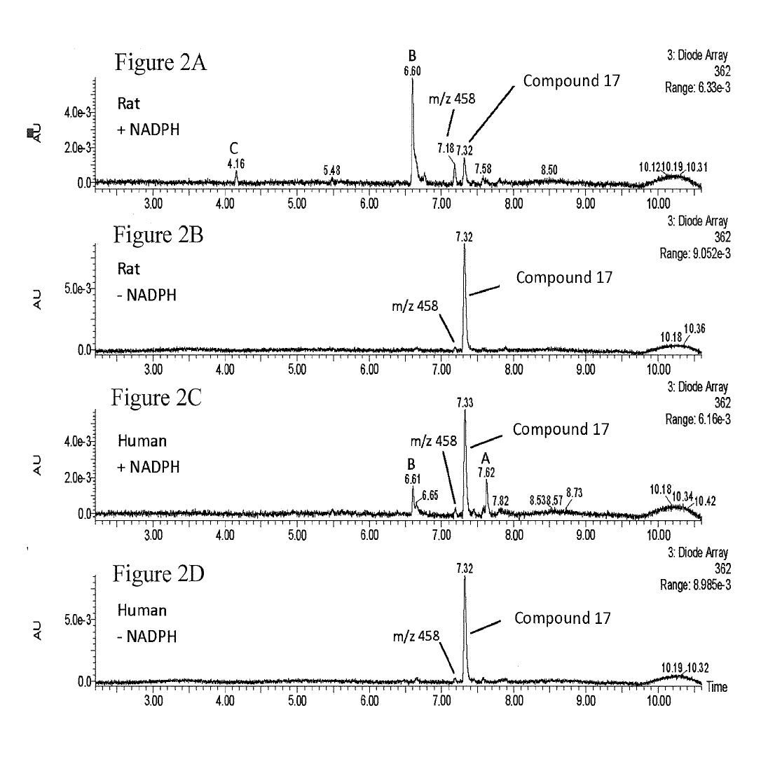

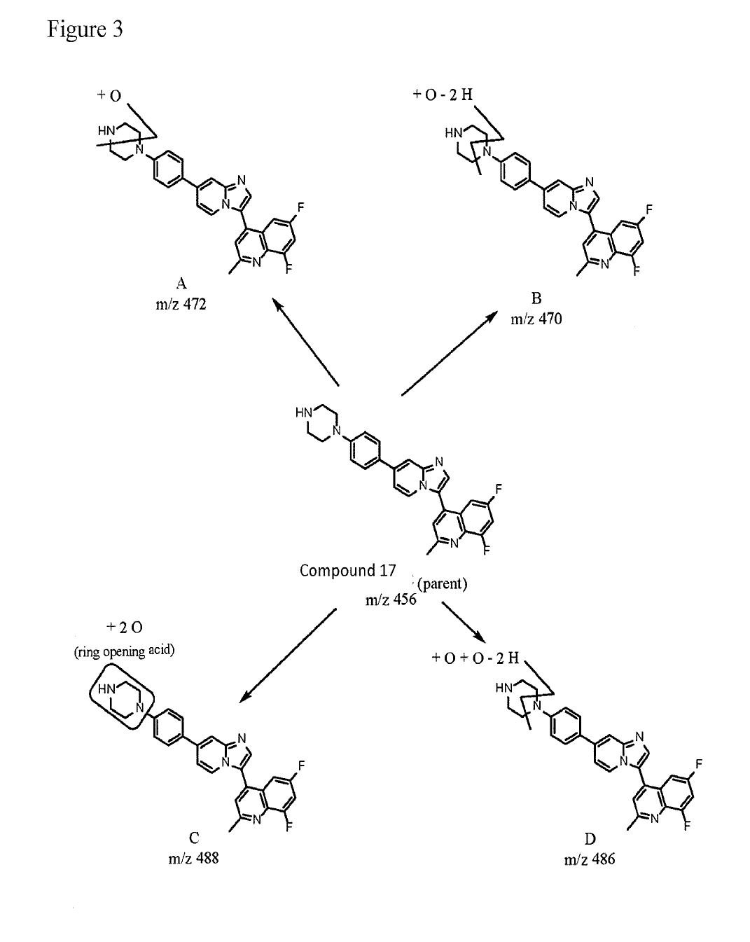

### DETAILED DESCRIPTION OF THE INVENTION

In certain aspects, the invention provides substituted imidazo[1,2-a]pyridine compounds, and pharmaceutical compositions thereof. In particular, such substituted compounds are useful as BMP inhibitors, and thus can be used to treat or prevent a disease or condition.

### I. Compounds

In certain embodiments, the invention relates to compounds having the structure of Formula (I), or a pharmaceutically acceptable salt thereof:

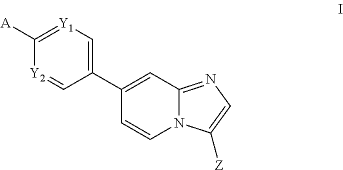

wherein
Y¹ and Y² are each independently CR¹ or N;
R¹ is, independently for each occurrence, H or alkyl;
A is optionally substituted alkoxy, cycloalkylalkoxy, heterocyclyl, heterocyclylalkoxy, or amino; and
Z is optionally substituted heteroaryl.

In certain embodiments of Formula I, Y¹ is N and Y² is CR¹. In other embodiments of Formula I, Y¹ and Y² are CR¹. In yet other embodiments of Formula I, Y¹ and Y² are N. In certain such embodiments, R¹ is H. In other such embodiments, R¹ is alkyl such as lower alkyl.

In certain embodiments of Formula I,
Z is

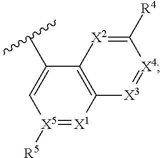

X¹, X², and X³ are each independently CR² or N, provided that at least one of X¹, X², and X³ is N;
X⁴ is CR³ or N;
X⁵ is C or N;
R², independently for each occurrence, is H, halo, or alkyl; and
R³, R⁴, and R⁵ are each independently H, halo, hydroxyl, cyano, optionally substituted alkyl, or optionally substituted alkoxy.

In some embodiments, X¹ is N. In some embodiments, X² is N. In some embodiments, X³ is N. In some embodiments, X⁴ is N. In some embodiments, X⁵ is N.

In certain embodiments of Formula I, Z is

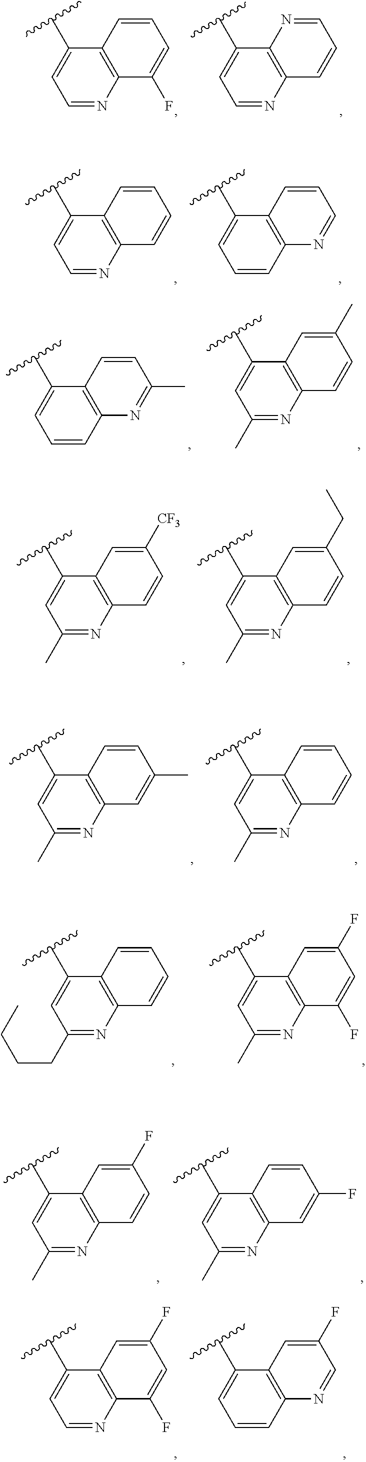

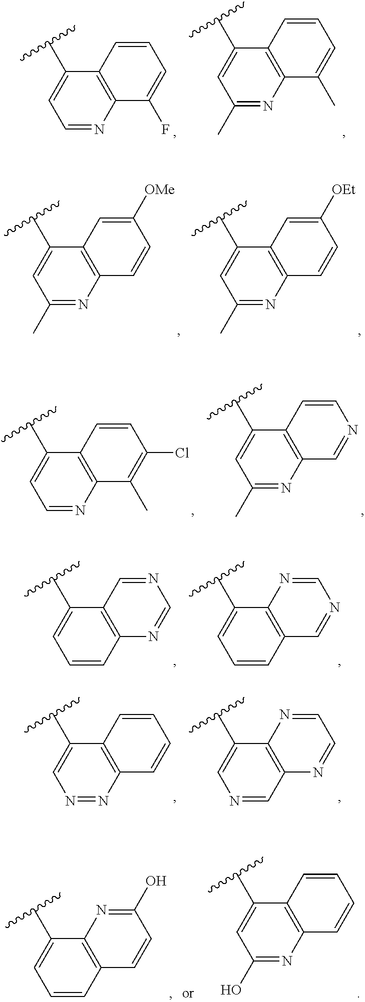

In certain embodiments of Formula I, Z is

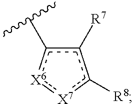

X⁶ and X⁷ are each independently CR⁵, S, or O, provided that one of X⁶ and X⁷ is CR⁵;
each R⁵ is independently H, halo, cyano, or optionally substituted alkyl, acyl, carboxy, or carbonyl; and
R⁷ and R⁸ are each independently H, halo, cyano, optionally substituted alkyl, or amide; or
R⁷ and R⁸ combine to form an optionally substituted 6-membered heteroaryl ring.

In some embodiments, X⁶ is S. In other embodiments, X⁷ is S. In yet other embodiments, X⁷ is O.

In certain embodiments of Formula I, Z is

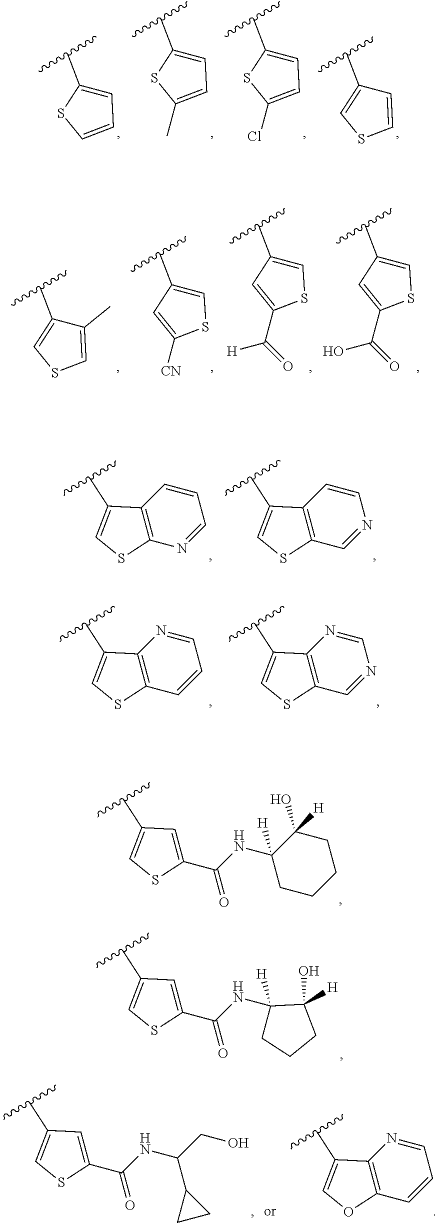

In certain embodiments of Formula I, Z is

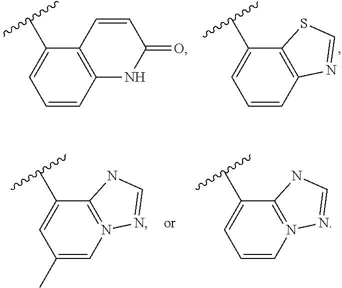

In certain embodiments of Formula I, A is optionally substituted alkoxy, cycloalkylalkoxy, or heterocyclalkoxy.

In certain embodiments of Formula I, A is

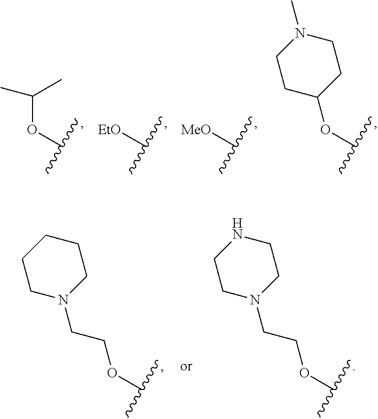

In certain embodiments of Formula I, A is amino, alkylamino, heteroalkylamino, cycloalkylamino, cycloalkylalkylamino, heterocyclylamino, or heterocycloalkylamino.

In certain embodiments of Formula I, A is an optionally substituted nitrogen-containing heterocyclyl.

In certain embodiments of Formula I,

### A is

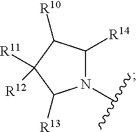

and
R¹⁰, R¹¹, R¹², R¹³, and R¹⁴ are each independently H, optionally substituted alkyl, or optionally substituted heterocyclyl, alkyl aminoalkyl, or heterocycloalkyl; or
R¹⁰ and R¹² combine to form an optionally substituted 5-membered ring; or
R¹⁰ and R¹⁴ combine to form an optionally substituted 5-membered ring; or
R¹¹ and R¹² combine to form an optionally substituted 4-, 5-, or 6-membered ring. In some embodiments, the optionally substituted 4-, 5-, or 6-membered ring comprises a heteroatom. In some embodiments, the heteroatom is N.

In certain embodiments of Formula I, A is

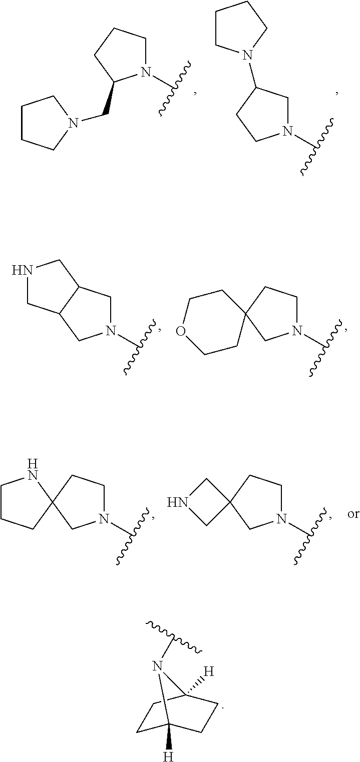

In certain embodiments of Formula I,

### A is

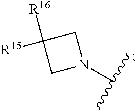

and
R¹⁵ and R¹⁶ are each independently H, halo, cyano, or alkyl; or
R¹⁵ and R¹⁶ combine to form an optionally substituted 4-, 5-, or 6-membered ring.

In certain embodiments of Formula I, A is

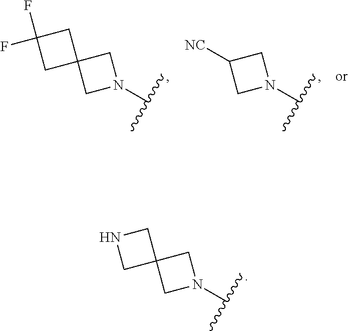 [SMILES: *N1CC2(C1)CC(F)(F)C2 ; *N1CC(C#N)C1 ; *N1CC2(CNC2)C1]

In certain embodiments of Formula I, A is

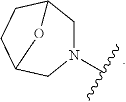 [SMILES: *N1CC2CCC(C1)O2]

In certain embodiments of Formula I, A is

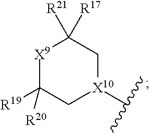

wherein
X⁹ is CHR¹⁸, NR¹⁸, or O;
X¹⁰ is CH or N;
R¹⁸ is H, optionally substituted alkyl, aryl, heteroaryl, cycloalkyl, heterocyclyl, cycloalkylalkyl, heterocycloalkyl, alkoxyalkyl, aralkyl, heteroaralkyl, —C(O)-alkyl, or sulfone; and
R²¹, R¹⁷, R¹⁸, and R¹⁹ are each independently H, halo, cyano, or alkyl; or
R²¹ and R¹⁸ combine to form an optionally substituted 4-, 5-, or 6-membered ring; or
R²¹ and R¹⁷ combine to form a carbonyl.

In some embodiments, X⁹ is N. In some embodiments, X¹⁰ is N. In some embodiments, X⁹ is O and R¹⁸ is absent.

In certain embodiments of Formula I, A is

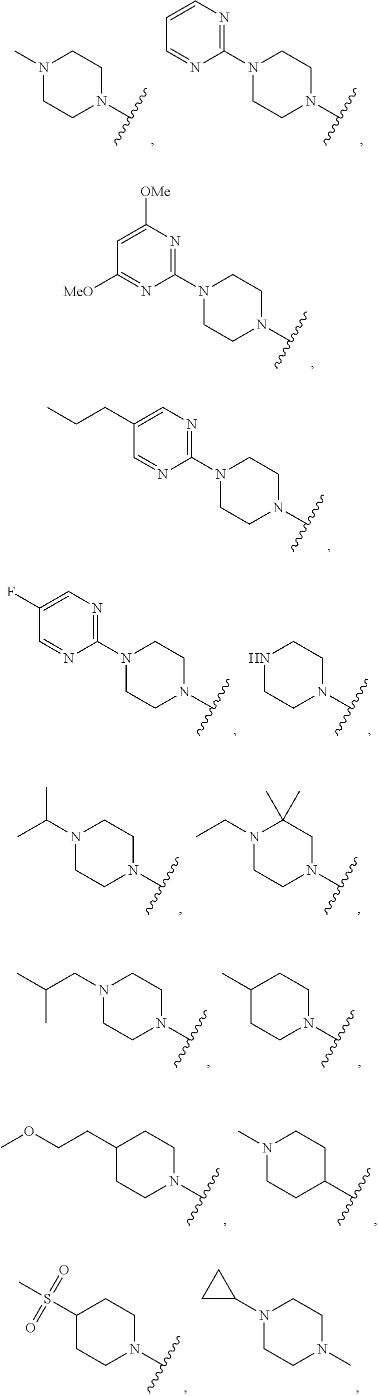

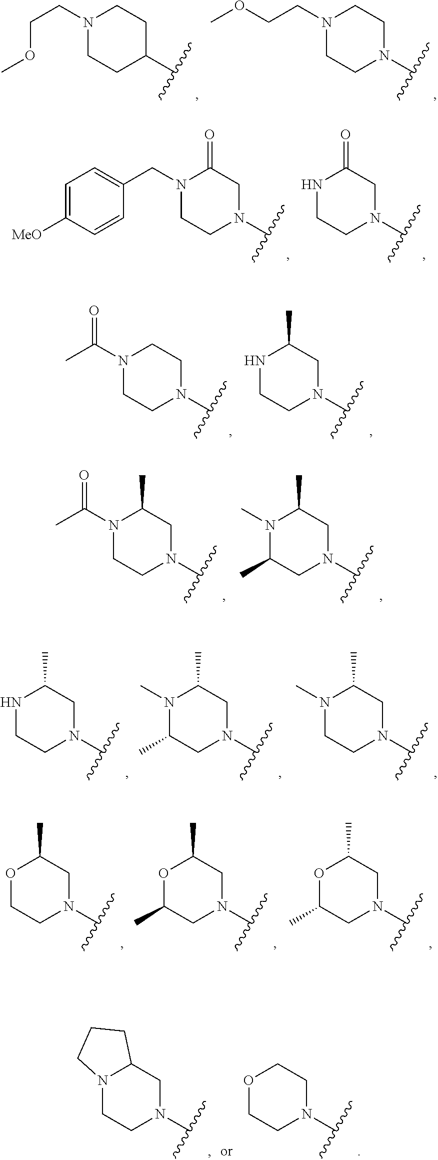

In another aspect, the invention relates to a compound selected from Table 1 or a pharmaceutically acceptable salt thereof, such as a compound having the structure:

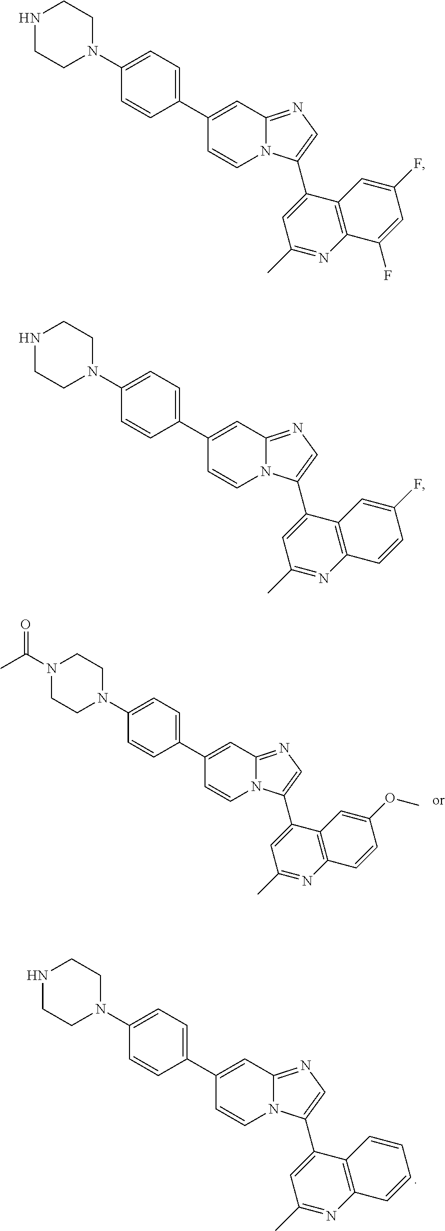 [SMILES: Cc1cc(-c2cnc3cc(-c4ccc(N5CCNCC5)cc4)ccn23)c2cc(F)cc(F)c2n1 ; Cc1cc(-c2cnc3cc(-c4ccc(N5CCNCC5)cc4)ccn23)c2cc(F)ccc2n1 ; COc1ccc2nc(C)cc(-c3cnc4cc(-c5ccc(N6CCN(C(C)=O)CC6)cc5)ccn34)c2c1 ; Cc1cc(-c2cnc3cc(-c4ccc(N5CCNCC5)cc4)ccn23)c2ccccc2n1]

In certain embodiments, compounds of the invention may be racemic. In certain embodiments, compounds of the invention may be enriched in one enantiomer. For example, a compound of the invention may have greater than 30% ee, 40% ee, 50% ee, 60% ee, 70% ee, 80% ee, 90% ee, or even 95% or greater ee. The compounds of the invention have more than one stereocenter. Consequently, compounds of the invention may be enriched in one or more diastereomer. For example, a compound of the invention may have greater than 30% de, 40% de, 50% de, 60% de, 70% de. 80% de, 90% de, or even 95% or greater de.

In certain embodiments, as will be described in detail below, the present invention relates to methods of treating or preventing a disease or condition with a compound of Formula I, or a pharmaceutically acceptable salt thereof. In certain embodiments, the therapeutic preparation may be enriched to provide predominantly one enantiomer of a compound of Formula I. An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, the therapeutic preparation may be enriched to provide predominantly one diastereomer of a compound of Formula I. A diastereomerically enriched mixture may comprise, for example, at least 60 mol percent of one diastereomer, or more preferably at least 75, 90, 95, or even 99 mol percent.

In certain embodiments, the present invention provides a pharmaceutical preparation suitable for use in a human patient in the treatment of a disease or condition, comprising an effective amount of any compound of Formula I, and one or more pharmaceutically acceptable excipients. In certain embodiments, the pharmaceutical preparations may be for use in treating or preventing a condition or disease as described herein. In certain embodiments, the pharmaceutical preparations have a low enough pyrogen activity to be suitable for use in a human patient.

Compounds of any of the above structures may be used in the manufacture of medicaments for the treatment of any diseases or conditions disclosed herein.

Exemplary compounds of Formula I are depicted in Table 1. The compounds of Table 1 are understood to encompass both the free base and the conjugate acid. For example, the compounds in Table 1 may be depicted as complexes or salts with trifluoroacetic acid or hydrochloric acid, but the compounds in their corresponding free base forms or as salts with other acids are equally within the scope of the invention. Compounds may be isolated in either the free base form, as a salt (e.g., a hydrochloride salt) or in both forms. In the chemical structures shown below, standard chemical abbreviations are sometimes used.

TABLE 1
Exemplary Compounds of Formula I

| Structure | Cmpd |
|---|---|
| 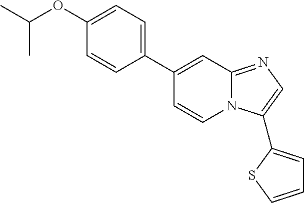 [SMILES: CC(C)Oc1ccc(-c2ccn3c(-c4cccs4)cnc3c2)cc1] | 1 |
| 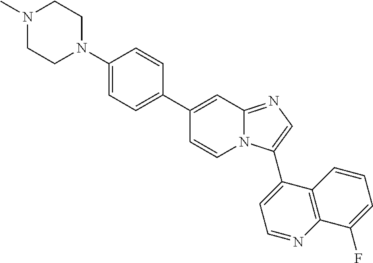 [SMILES: CN1CCN(c2ccc(-c3ccn4c(-c5ccnc6c(F)cccc56)cnc4c3)cc2)CC1] | 2 |
| 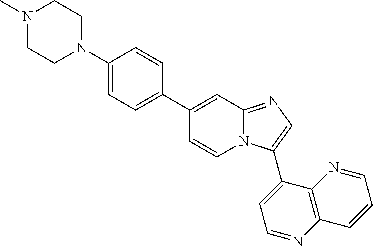 [SMILES: CN1CCN(c2ccc(-c3ccn4c(-c5ccnc6cccnc56)cnc4c3)cc2)CC1] | 3 |
| 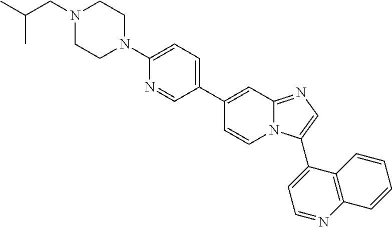 [SMILES: CC(C)CN1CCN(c2ccc(-c3ccn4c(-c5ccnc6ccccc56)cnc4c3)cn2)CC1] | 4 |
| 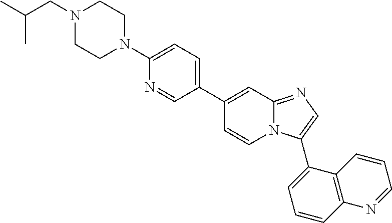 [SMILES: CC(C)CN1CCN(c2ccc(-c3ccn4c(-c5cccc6ncccc56)cnc4c3)cn2)CC1] | 5 |
| 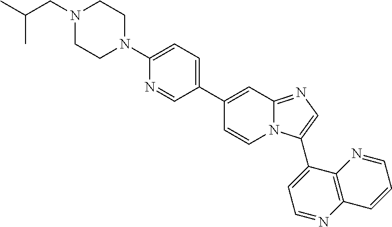 [SMILES: CC(C)CN1CCN(c2ccc(-c3ccn4c(-c5ccnc6cccnc56)cnc4c3)cn2)CC1] | 6 |
| 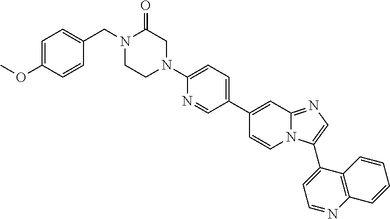 [SMILES: COc1ccc(CN2CCN(c3ccc(-c4ccn5c(-c6ccnc7ccccc67)cnc5c4)cn3)CC2=O)cc1] | 7 |
| 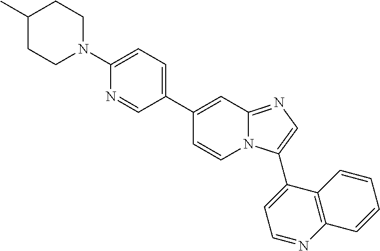 [SMILES: CC1CCN(c2ccc(-c3ccn4c(-c5ccnc6ccccc56)cnc4c3)cn2)CC1] | 8 |
| 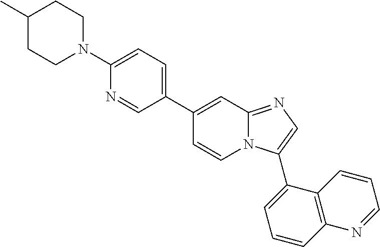 [SMILES: CC1CCN(c2ccc(-c3ccn4c(-c5cccc6ncccc56)cnc4c3)cn2)CC1] | 9 |
| 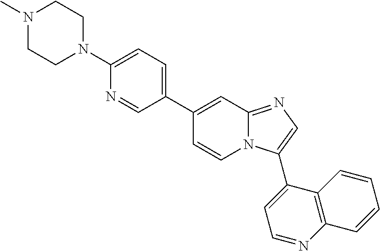 [SMILES: CN1CCN(c2ccc(-c3ccn4c(-c5ccnc6ccccc56)cnc4c3)cn2)CC1] | 10 |
| 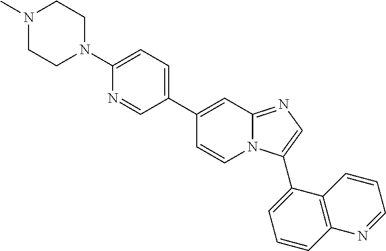 [SMILES: CN1CCN(c2ccc(-c3ccn4c(-c5cccc6ncccc56)cnc4c3)cn2)CC1] | 11 |
| 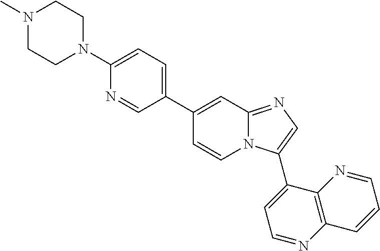 [SMILES: CN1CCN(c2ccc(-c3ccn4c(-c5ccnc6cccnc56)cnc4c3)cn2)CC1] | 12 |
| 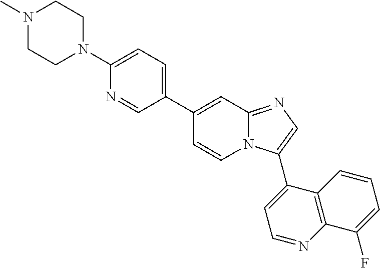 [SMILES: CN1CCN(c2ccc(-c3ccn4c(-c5ccnc6c(F)cccc56)cnc4c3)cn2)CC1] | 13 |
| 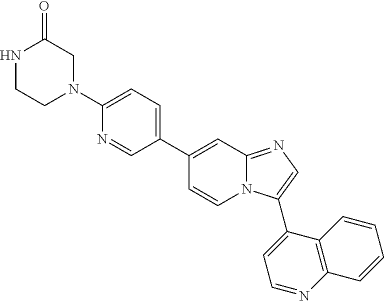 [SMILES: O=C1CN(c2ccc(-c3ccn4c(-c5ccnc6ccccc56)cnc4c3)cn2)CCN1] | 14 |
| 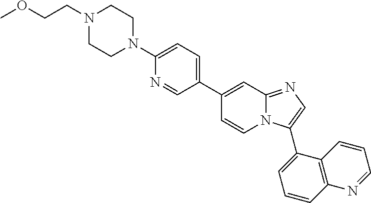 [SMILES: COCCN1CCN(c2ccc(-c3ccn4c(-c5cccc6ncccc56)cnc4c3)cn2)CC1] | 15 |
| 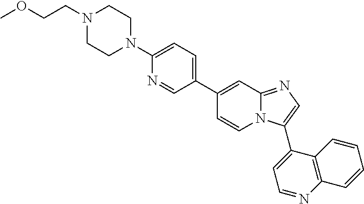 [SMILES: COCCN1CCN(c2ccc(-c3ccn4c(-c5ccnc6ccccc56)cnc4c3)cn2)CC1] | 16 |
| 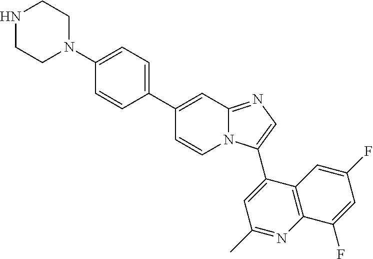 [SMILES: Cc1cc(-c2cnc3cc(-c4ccc(N5CCNCC5)cc4)ccn23)c2cc(F)cc(F)c2n1] | 17 |
| 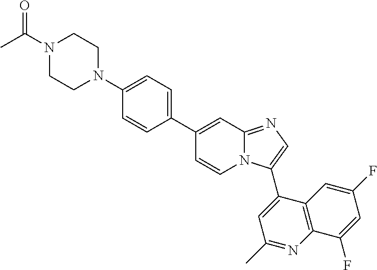 [SMILES: CC(=O)N1CCN(c2ccc(-c3ccn4c(-c5cc(C)nc6c(F)cc(F)cc56)cnc4c3)cc2)CC1] | 18 |
| 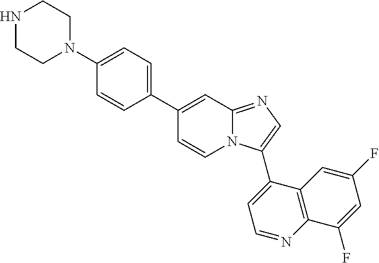 [SMILES: Fc1cc(F)c2nccc(-c3cnc4cc(-c5ccc(N6CCNCC6)cc5)ccn34)c2c1] | 19 |
| 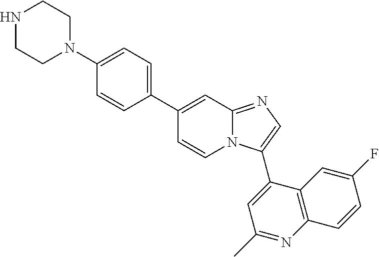 [SMILES: Cc1cc(-c2cnc3cc(-c4ccc(N5CCNCC5)cc4)ccn23)c2cc(F)ccc2n1] | 20 |
| 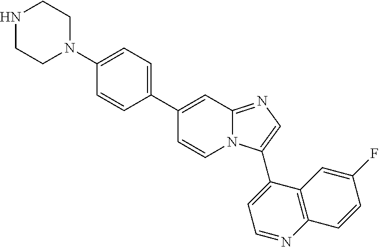 [SMILES: Fc1ccc2nccc(-c3cnc4cc(-c5ccc(N6CCNCC6)cc5)ccn34)c2c1] | 21 |
| 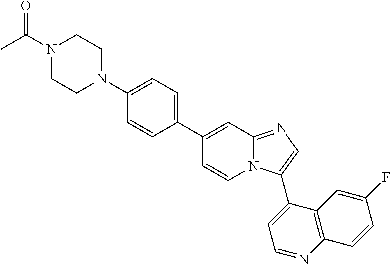 [SMILES: CC(=O)N1CCN(c2ccc(-c3ccn4c(-c5ccnc6ccc(F)cc56)cnc4c3)cc2)CC1] | 22 |
| 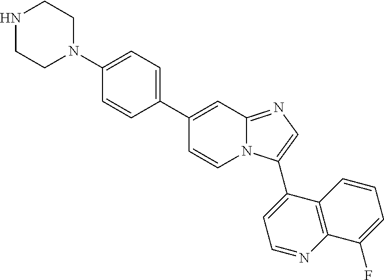 [SMILES: Fc1cccc2c(-c3cnc4cc(-c5ccc(N6CCNCC6)cc5)ccn34)ccnc12] | 23 |
| 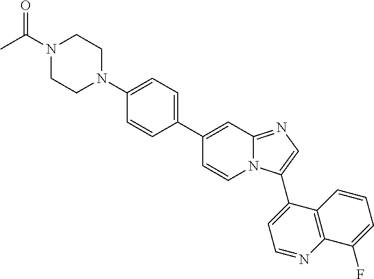 [SMILES: CC(=O)N1CCN(c2ccc(-c3ccn4c(-c5ccnc6c(F)cccc56)cnc4c3)cc2)CC1] | 24 |
| 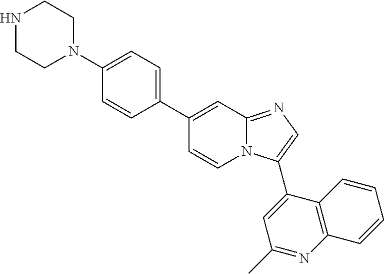 [SMILES: Cc1cc(-c2cnc3cc(-c4ccc(N5CCNCC5)cc4)ccn23)c2ccccc2n1] | 25 |
| 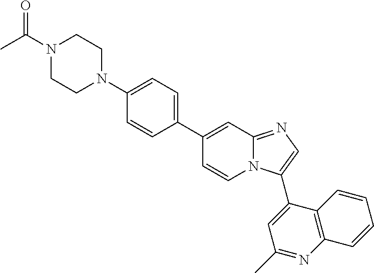 [SMILES: CC(=O)N1CCN(c2ccc(-c3ccn4c(-c5cc(C)nc6ccccc56)cnc4c3)cc2)CC1] | 26 |
| 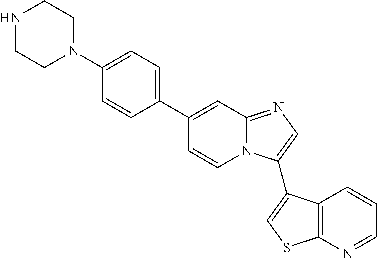 [SMILES: c1cnc2scc(-c3cnc4cc(-c5ccc(N6CCNCC6)cc5)ccn34)c2c1] | 27 |
| 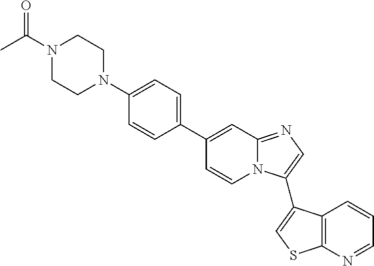 [SMILES: CC(=O)N1CCN(c2ccc(-c3ccn4c(-c5csc6ncccc56)cnc4c3)cc2)CC1] | 28 |
| 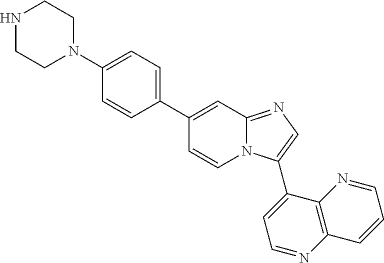 [SMILES: c1cnc2c(-c3cnc4cc(-c5ccc(N6CCNCC6)cc5)ccn34)ccnc2c1] | 29 |
| 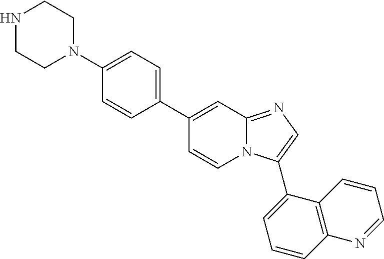 [SMILES: c1cc(-c2cnc3cc(-c4ccc(N5CCNCC5)cc4)ccn23)c2cccnc2c1] | 30 |
| 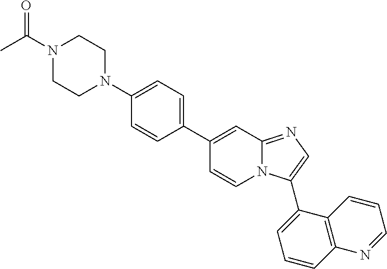 [SMILES: CC(=O)N1CCN(c2ccc(-c3ccn4c(-c5cccc6ncccc56)cnc4c3)cc2)CC1] | 31 |
| 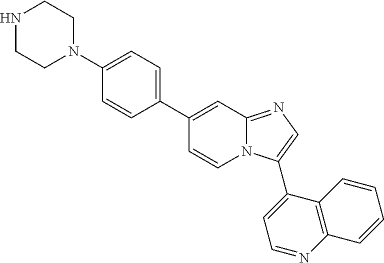 [SMILES: c1ccc2c(-c3cnc4cc(-c5ccc(N6CCNCC6)cc5)ccn34)ccnc2c1] | 32 |
| 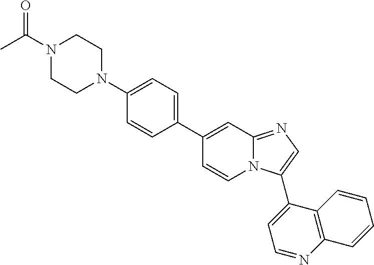 [SMILES: CC(=O)N1CCN(c2ccc(-c3ccn4c(-c5ccnc6ccccc56)cnc4c3)cc2)CC1] | 33 |
| 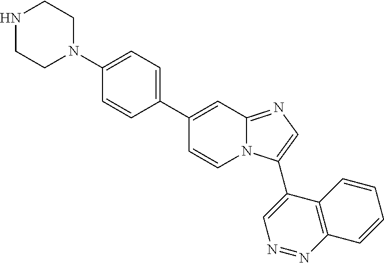 [SMILES: c1ccc2c(-c3cnc4cc(-c5ccc(N6CCNCC6)cc5)ccn34)cnnc2c1] | 34 |
| 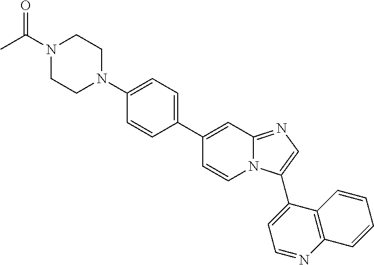 [SMILES: CC(=O)N1CCN(c2ccc(-c3ccn4c(-c5ccnc6ccccc56)cnc4c3)cc2)CC1] | 35 |
| 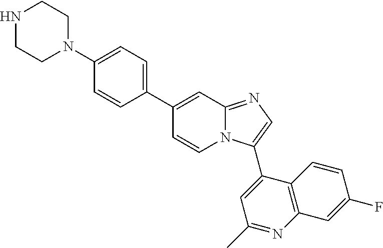 [SMILES: Cc1cc(-c2cnc3cc(-c4ccc(N5CCNCC5)cc4)ccn23)c2ccc(F)cc2n1] | 36 |
| 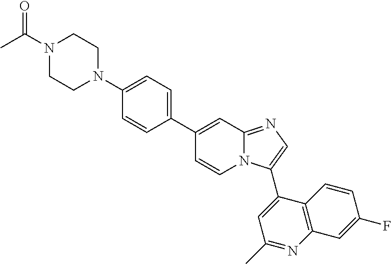 [SMILES: CC(=O)N1CCN(c2ccc(-c3ccn4c(-c5cc(C)nc6cc(F)ccc56)cnc4c3)cc2)CC1] | 37 |
| 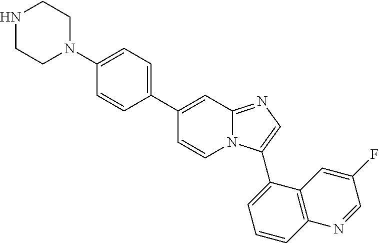 [SMILES: Fc1cnc2cccc(-c3cnc4cc(-c5ccc(N6CCNCC6)cc5)ccn34)c2c1] | 38 |
| 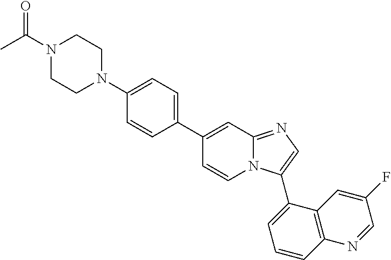 [SMILES: CC(=O)N1CCN(c2ccc(-c3ccn4c(-c5cccc6ncc(F)cc56)cnc4c3)cc2)CC1] | 39 |
| 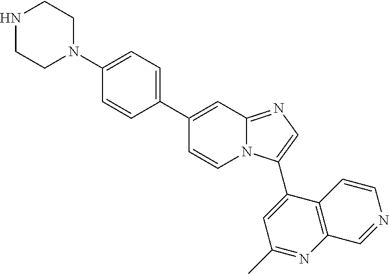 [SMILES: Cc1cc(-c2cnc3cc(-c4ccc(N5CCNCC5)cc4)ccn23)c2ccncc2n1] | 40 |
| 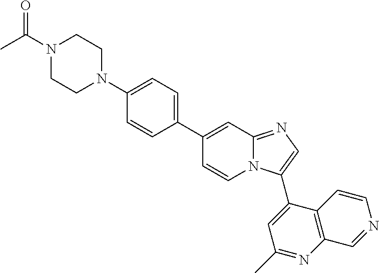 [SMILES: CC(=O)N1CCN(c2ccc(-c3ccn4c(-c5cc(C)nc6cnccc56)cnc4c3)cc2)CC1] | 41 |
| 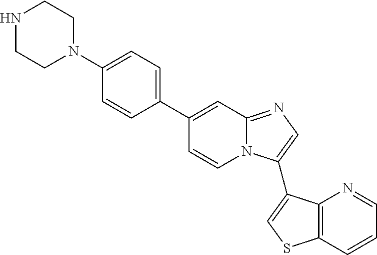 [SMILES: c1cnc2c(-c3cnc4cc(-c5ccc(N6CCNCC6)cc5)ccn34)csc2c1] | 42 |
| 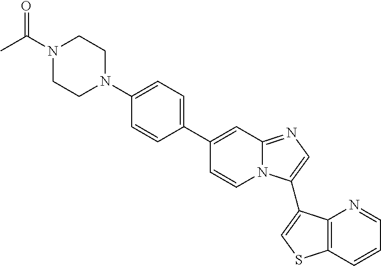 [SMILES: CC(=O)N1CCN(c2ccc(-c3ccn4c(-c5csc6cccnc56)cnc4c3)cc2)CC1] | 43 |
| 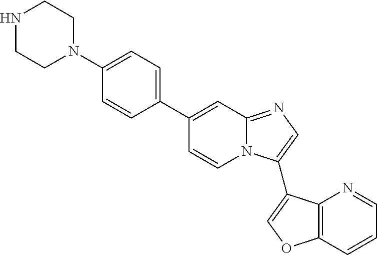 [SMILES: c1cnc2c(-c3cnc4cc(-c5ccc(N6CCNCC6)cc5)ccn34)coc2c1] | 44 |
| 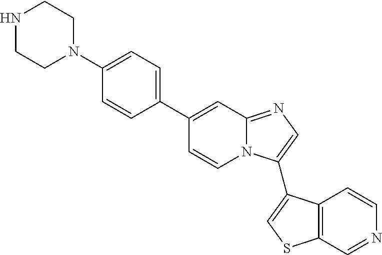 [SMILES: c1cc2c(-c3cnc4cc(-c5ccc(N6CCNCC6)cc5)ccn34)csc2cn1] | 45 |
| 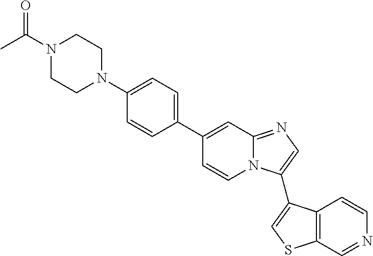 [SMILES: CC(=O)N1CCN(c2ccc(-c3ccn4c(-c5csc6cnccc56)cnc4c3)cc2)CC1] | 46 |
| 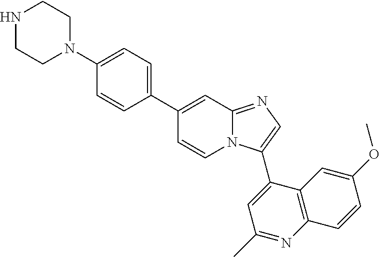 [SMILES: COc1ccc2nc(C)cc(-c3cnc4cc(-c5ccc(N6CCNCC6)cc5)ccn34)c2c1] | 47 |
| 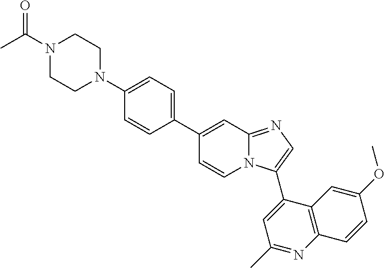 [SMILES: COc1ccc2nc(C)cc(-c3cnc4cc(-c5ccc(N6CCN(C(C)=O)CC6)cc5)ccn34)c2c1] | 48 |
| 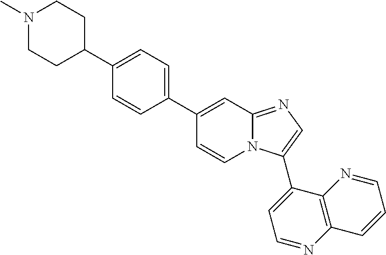 [SMILES: CN1CCC(c2ccc(-c3ccn4c(-c5ccnc6cccnc56)cnc4c3)cc2)CC1] | 49 |
| 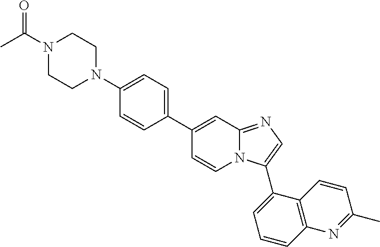 [SMILES: CC(=O)N1CCN(c2ccc(-c3ccn4c(-c5cccc6nc(C)ccc56)cnc4c3)cc2)CC1] | 50 |
| 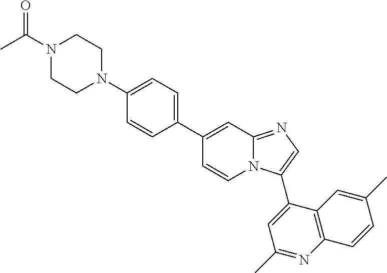 [SMILES: CC(=O)N1CCN(c2ccc(-c3ccn4c(-c5cc(C)nc6ccc(C)cc56)cnc4c3)cc2)CC1] | 51 |
| 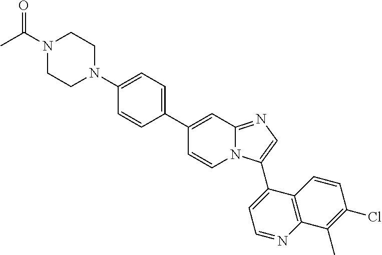 [SMILES: CC(=O)N1CCN(c2ccc(-c3ccn4c(-c5ccnc6c(C)c(Cl)ccc56)cnc4c3)cc2)CC1] | 52 |
| 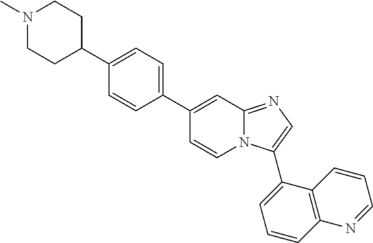 [SMILES: CN1CCC(c2ccc(-c3ccn4c(-c5cccc6ncccc56)cnc4c3)cc2)CC1] | 53 |
| 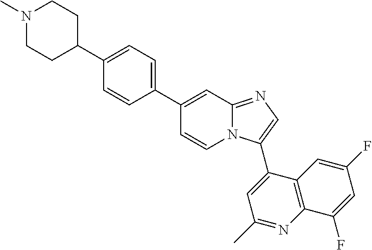 [SMILES: Cc1cc(-c2cnc3cc(-c4ccc(C5CCN(C)CC5)cc4)ccn23)c2cc(F)cc(F)c2n1] | 54 |
| 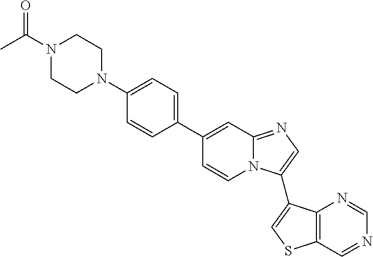 [SMILES: CC(=O)N1CCN(c2ccc(-c3ccn4c(-c5csc6cncnc56)cnc4c3)cc2)CC1] | 55 |
| 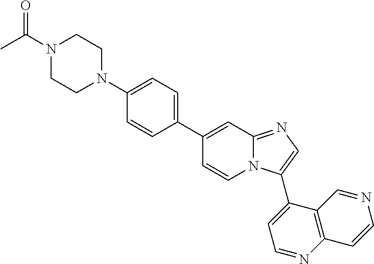 [SMILES: CC(=O)N1CCN(c2ccc(-c3ccn4c(-c5ccnc6ccncc56)cnc4c3)cc2)CC1] | 56 |
| 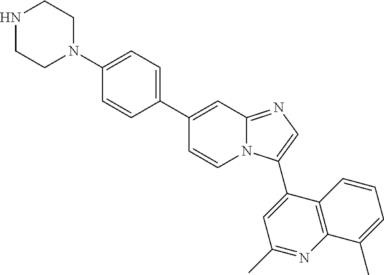 [SMILES: Cc1cc(-c2cnc3cc(-c4ccc(N5CCNCC5)cc4)ccn23)c2cccc(C)c2n1] | 57 |
| 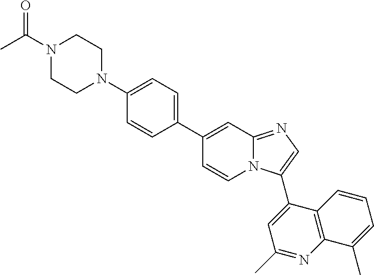 [SMILES: CC(=O)N1CCN(c2ccc(-c3ccn4c(-c5cc(C)nc6c(C)cccc56)cnc4c3)cc2)CC1] | 58 |
| 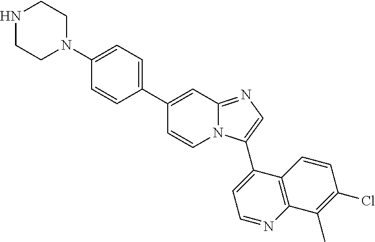 [SMILES: Cc1c(Cl)ccc2c(-c3cnc4cc(-c5ccc(N6CCNCC6)cc5)ccn34)ccnc12] | 59 |
| 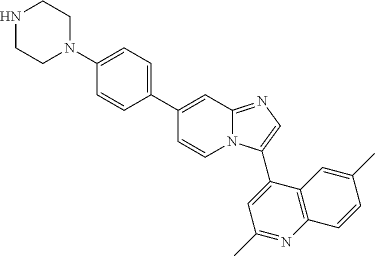 [SMILES: Cc1ccc2nc(C)cc(-c3cnc4cc(-c5ccc(N6CCNCC6)cc5)ccn34)c2c1] | 60 |
| 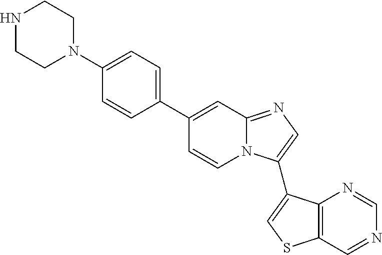 [SMILES: c1ncc2scc(-c3cnc4cc(-c5ccc(N6CCNCC6)cc5)ccn34)c2n1] | 61 |
| 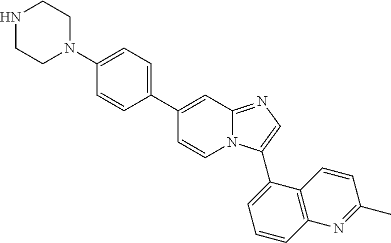 [SMILES: Cc1ccc2c(-c3cnc4cc(-c5ccc(N6CCNCC6)cc5)ccn34)cccc2n1] | 62 |
| 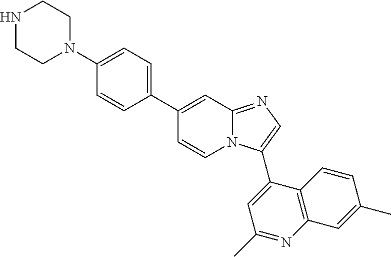 [SMILES: Cc1ccc2c(-c3cnc4cc(-c5ccc(N6CCNCC6)cc5)ccn34)cc(C)nc2c1] | 63 |
| 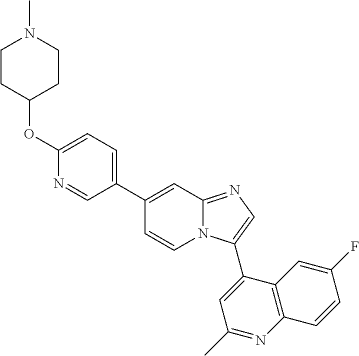 [SMILES: Cc1cc(-c2cnc3cc(-c4ccc(OC5CCN(C)CC5)nc4)ccn23)c2cc(F)ccc2n1] | 64 |
| 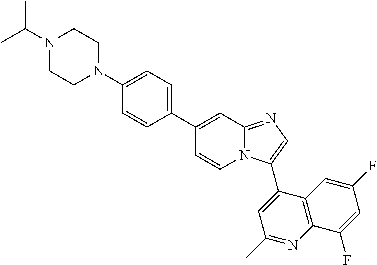 [SMILES: Cc1cc(-c2cnc3cc(-c4ccc(N5CCN(C(C)C)CC5)cc4)ccn23)c2cc(F)cc(F)c2n1] | 65 |
| 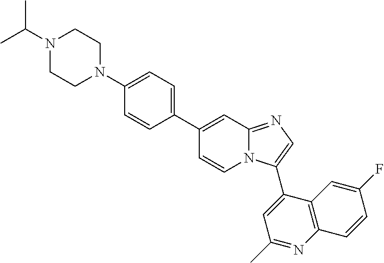 [SMILES: Cc1cc(-c2cnc3cc(-c4ccc(N5CCN(C(C)C)CC5)cc4)ccn23)c2cc(F)ccc2n1] | 66 |
| 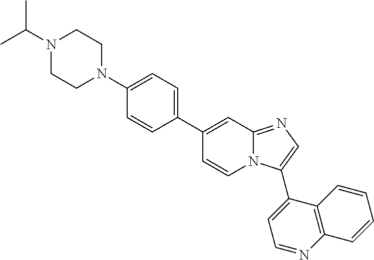 [SMILES: CC(C)N1CCN(c2ccc(-c3ccn4c(-c5ccnc6ccccc56)cnc4c3)cc2)CC1] | 67 |
| 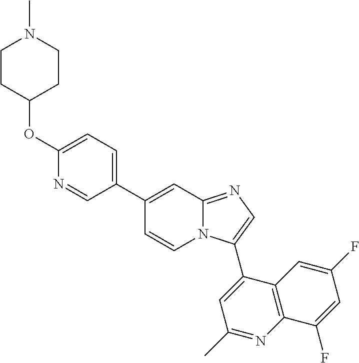 [SMILES: Cc1cc(-c2cnc3cc(-c4ccc(OC5CCN(C)CC5)nc4)ccn23)c2cc(F)cc(F)c2n1] | 68 |
| 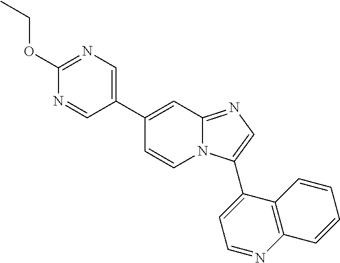 [SMILES: CCOc1ncc(-c2ccn3c(-c4ccnc5ccccc45)cnc3c2)cn1] | 69 |
| 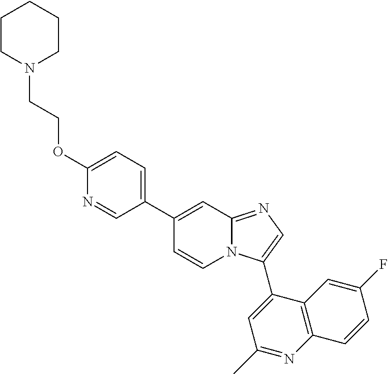 [SMILES: Cc1cc(-c2cnc3cc(-c4ccc(OCCN5CCCCC5)nc4)ccn23)c2cc(F)ccc2n1] | 70 |
| 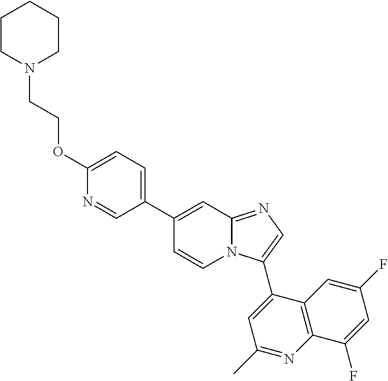 [SMILES: Cc1cc(-c2cnc3cc(-c4ccc(OCCN5CCCCC5)nc4)ccn23)c2cc(F)cc(F)c2n1] | 71 |
| 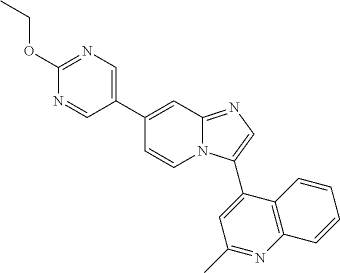 [SMILES: CCOc1ncc(-c2ccn3c(-c4cc(C)nc5ccccc45)cnc3c2)cn1] | 72 |
| 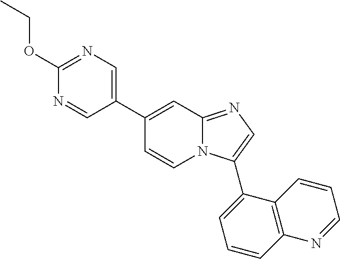 [SMILES: CCOc1ncc(-c2ccn3c(-c4cccc5ncccc45)cnc3c2)cn1] | 73 |
| 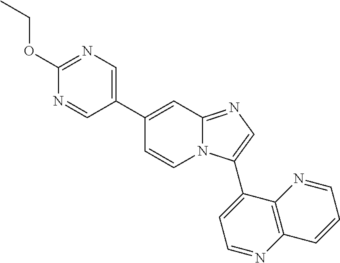 [SMILES: CCOc1ncc(-c2ccn3c(-c4ccnc5cccnc45)cnc3c2)cn1] | 74 |
| 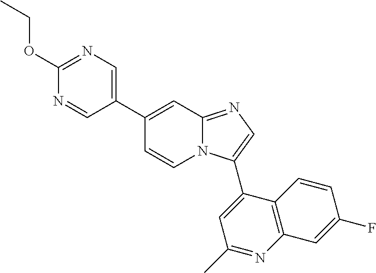 [SMILES: CCOc1ncc(-c2ccn3c(-c4cc(C)nc5cc(F)ccc45)cnc3c2)cn1] | 75 |
| 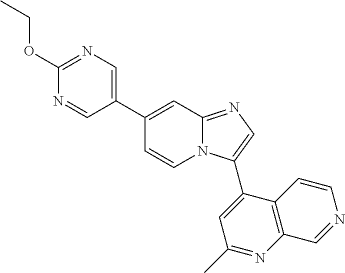 [SMILES: CCOc1ncc(-c2ccn3c(-c4cc(C)nc5cnccc45)cnc3c2)cn1] | 76 |
| 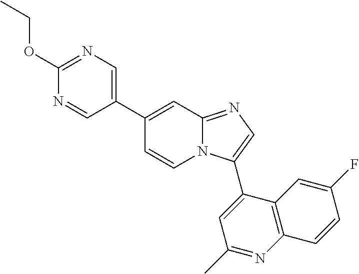 [SMILES: CCOc1ncc(-c2ccn3c(-c4cc(C)nc5ccc(F)cc45)cnc3c2)cn1] | 77 |
| 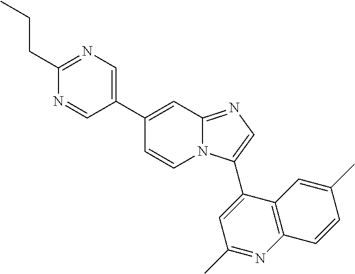 [SMILES: CCCc1ncc(-c2ccn3c(-c4cc(C)nc5ccc(C)cc45)cnc3c2)cn1] | 78 |
| 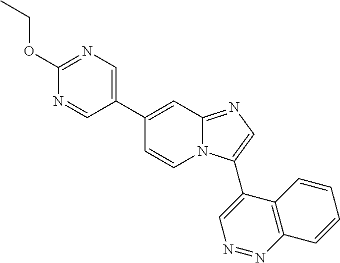 [SMILES: CCOc1ncc(-c2ccn3c(-c4cnnc5ccccc45)cnc3c2)cn1] | 79 |
| 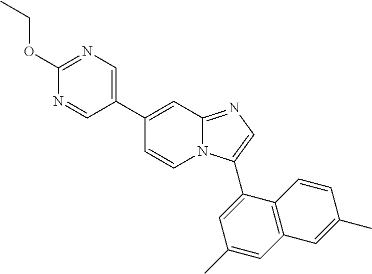 [SMILES: CCOc1ncc(-c2ccn3c(-c4cc(C)cc5cc(C)ccc45)cnc3c2)cn1] | 80 |
| 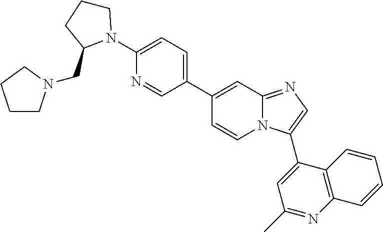 [SMILES: Cc1cc(-c2cnc3cc(-c4ccc(N5CCC[C@@H]5CN5CCCC5)nc4)ccn23)c2ccccc2n1] | 81 |
| 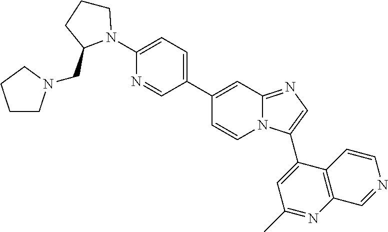 [SMILES: Cc1cc(-c2cnc3cc(-c4ccc(N5CCC[C@@H]5CN5CCCC5)nc4)ccn23)c2ccncc2n1] | 82 |
| 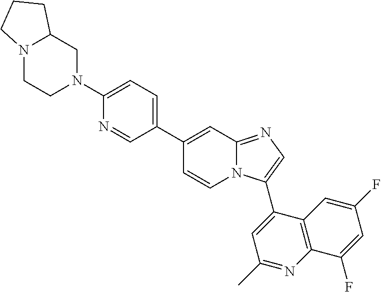 [SMILES: Cc1cc(-c2cnc3cc(-c4ccc(N5CCN6CCCC6C5)nc4)ccn23)c2cc(F)cc(F)c2n1] | 83 |
| 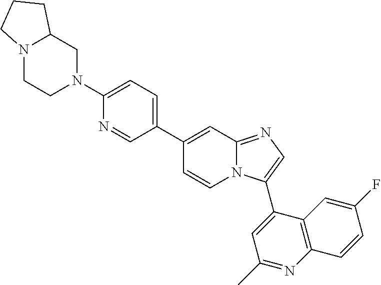 [SMILES: Cc1cc(-c2cnc3cc(-c4ccc(N5CCN6CCCC6C5)nc4)ccn23)c2cc(F)ccc2n1] | 84 |
| 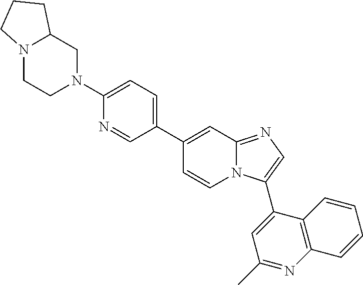 [SMILES: Cc1cc(-c2cnc3cc(-c4ccc(N5CCN6CCCC6C5)nc4)ccn23)c2ccccc2n1] | 85 |
| 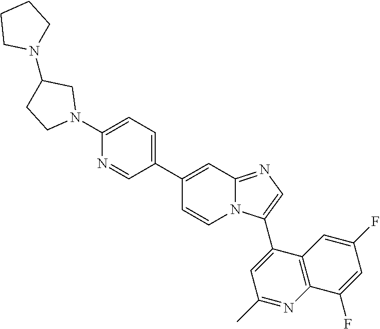 [SMILES: Cc1cc(-c2cnc3cc(-c4ccc(N5CCC(N6CCCC6)C5)nc4)ccn23)c2cc(F)cc(F)c2n1] | 86 |
| 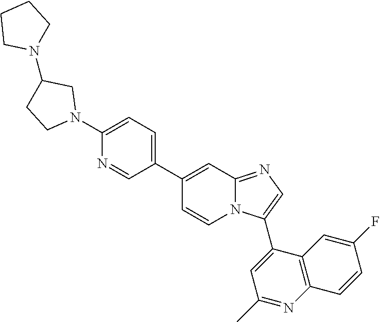 [SMILES: Cc1cc(-c2cnc3cc(-c4ccc(N5CCC(N6CCCC6)C5)nc4)ccn23)c2cc(F)ccc2n1] | 87 |
| 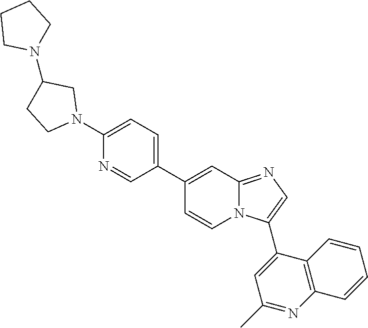 [SMILES: Cc1cc(-c2cnc3cc(-c4ccc(N5CCC(N6CCCC6)C5)nc4)ccn23)c2ccccc2n1] | 88 |
| 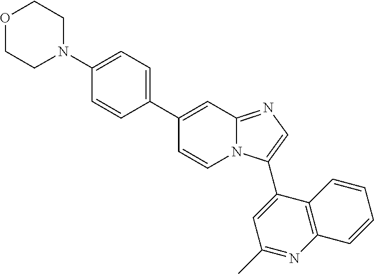 [SMILES: Cc1cc(-c2cnc3cc(-c4ccc(N5CCOCC5)cc4)ccn23)c2ccccc2n1] | 89 |
| 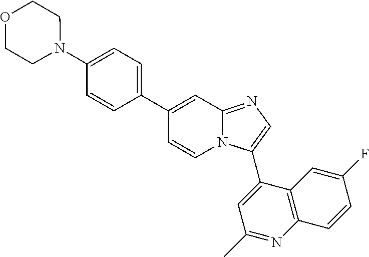 [SMILES: Cc1cc(-c2cnc3cc(-c4ccc(N5CCOCC5)cc4)ccn23)c2cc(F)ccc2n1] | 90 |
| 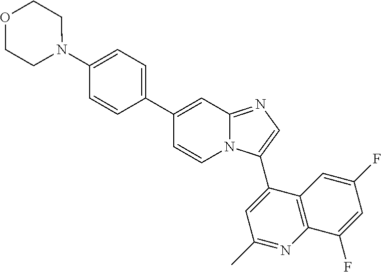 [SMILES: Cc1cc(-c2cnc3cc(-c4ccc(N5CCOCC5)cc4)ccn23)c2cc(F)cc(F)c2n1] | 91 |
| 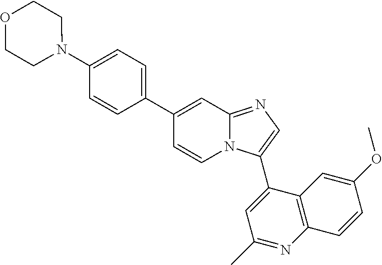 [SMILES: COc1ccc2nc(C)cc(-c3cnc4cc(-c5ccc(N6CCOCC6)cc5)ccn34)c2c1] | 92 |
| 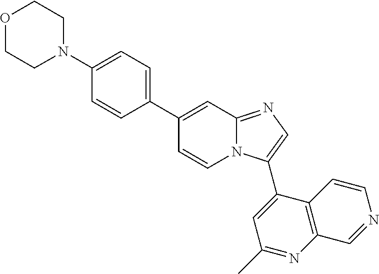 [SMILES: Cc1cc(-c2cnc3cc(-c4ccc(N5CCOCC5)cc4)ccn23)c2ccncc2n1] | 93 |
| 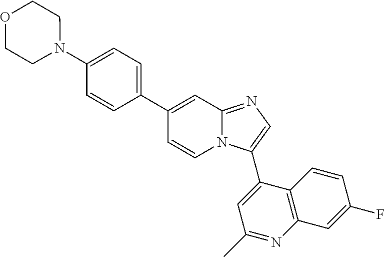 [SMILES: Cc1cc(-c2cnc3cc(-c4ccc(N5CCOCC5)cc4)ccn23)c2ccc(F)cc2n1] | 94 |
| 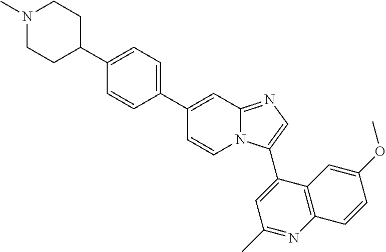 [SMILES: COc1ccc2nc(C)cc(-c3cnc4cc(-c5ccc(C6CCN(C)CC6)cc5)ccn34)c2c1] | 95 |
| 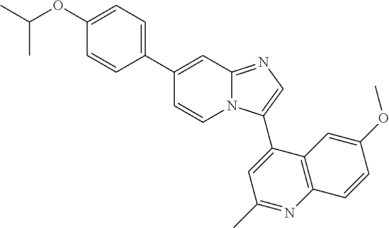 [SMILES: COc1ccc2nc(C)cc(-c3cnc4cc(-c5ccc(OC(C)C)cc5)ccn34)c2c1] | 96 |
| 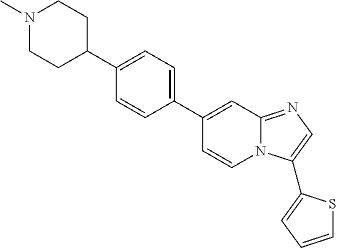 [SMILES: CN1CCC(c2ccc(-c3ccn4c(-c5cccs5)cnc4c3)cc2)CC1] | 97 |
| 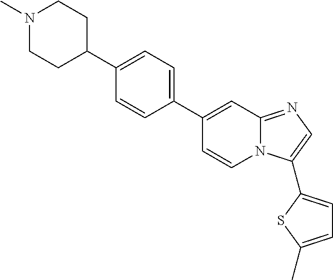 [SMILES: Cc1ccc(-c2cnc3cc(-c4ccc(C5CCN(C)CC5)cc4)ccn23)s1] | 98 |
| 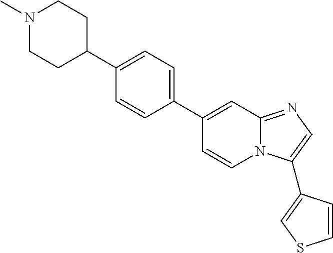 [SMILES: CN1CCC(c2ccc(-c3ccn4c(-c5ccsc5)cnc4c3)cc2)CC1] | 99 |
| 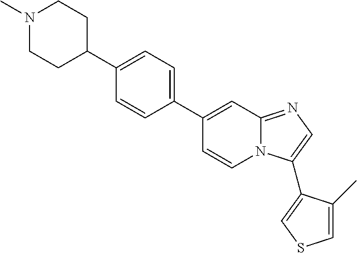 | 100 |
| 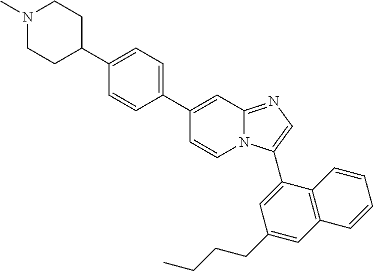 [SMILES: CCCCc1cc(-c2cnc3cc(-c4ccc(C5CCN(C)CC5)cc4)ccn23)c2ccccc2c1] | 101 |
| 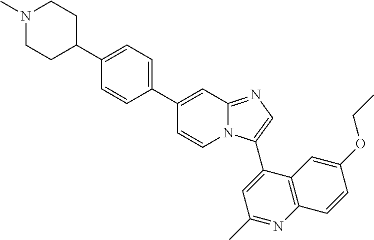 [SMILES: CCOc1ccc2nc(C)cc(-c3cnc4cc(-c5ccc(C6CCN(C)CC6)cc5)ccn34)c2c1] | 102 |
| 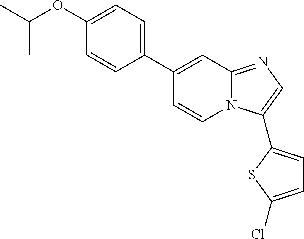 [SMILES: CC(C)Oc1ccc(-c2ccn3c(-c4ccc(Cl)s4)cnc3c2)cc1] | 103 |
| 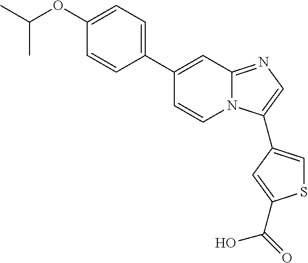 [SMILES: CC(C)Oc1ccc(-c2ccn3c(-c4csc(C(=O)O)c4)cnc3c2)cc1] | 104 |
| 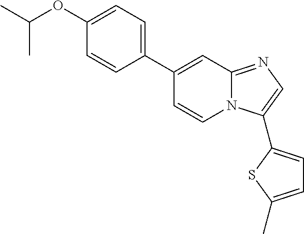 [SMILES: Cc1ccc(-c2cnc3cc(-c4ccc(OC(C)C)cc4)ccn23)s1] | 105 |
| 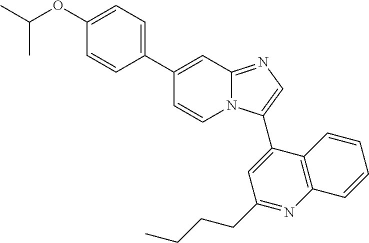 [SMILES: CCCCc1cc(-c2cnc3cc(-c4ccc(OC(C)C)cc4)ccn23)c2ccccc2n1] | 106 |
| 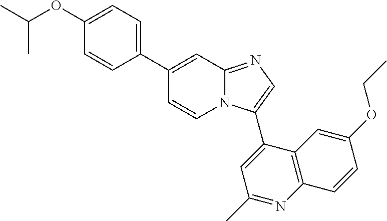 [SMILES: CCOc1ccc2nc(C)cc(-c3cnc4cc(-c5ccc(OC(C)C)cc5)ccn34)c2c1] | 107 |
| 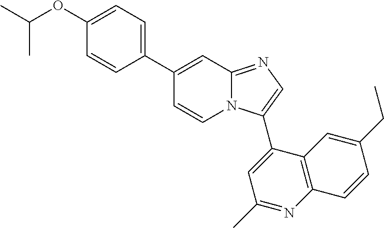 [SMILES: CCc1ccc2nc(C)cc(-c3cnc4cc(-c5ccc(OC(C)C)cc5)ccn34)c2c1] | 108 |
| 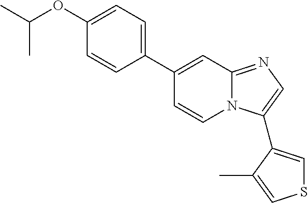 [SMILES: Cc1cscc1-c1cnc2cc(-c3ccc(OC(C)C)cc3)ccn12] | 109 |
| 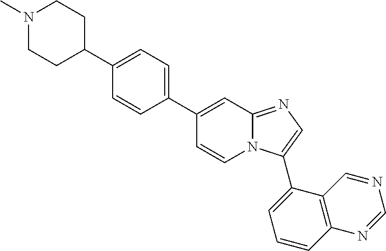 [SMILES: CN1CCC(c2ccc(-c3ccn4c(-c5cccc6ncncc56)cnc4c3)cc2)CC1] | 110 |
| 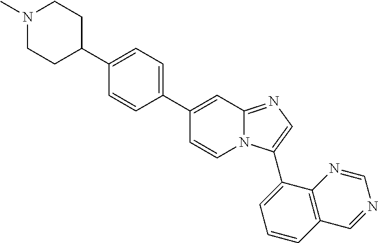 [SMILES: CN1CCC(c2ccc(-c3ccn4c(-c5cccc6cncnc56)cnc4c3)cc2)CC1] | 111 |
| 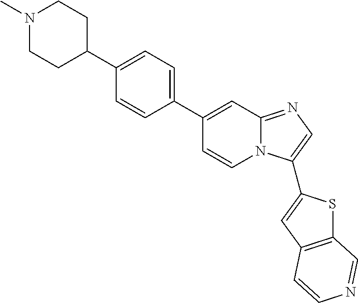 [SMILES: CN1CCC(c2ccc(-c3ccn4c(-c5cc6ccncc6s5)cnc4c3)cc2)CC1] | 112 |
| 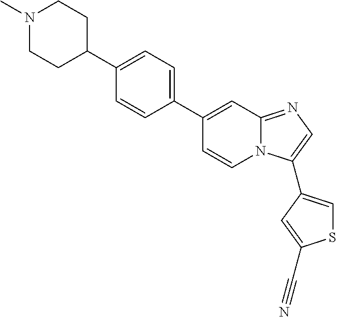 [SMILES: CN1CCC(c2ccc(-c3ccn4c(-c5csc(C#N)c5)cnc4c3)cc2)CC1] | 113 |
| 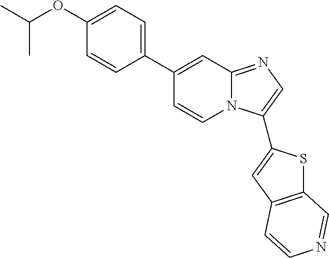 [SMILES: CC(C)Oc1ccc(-c2ccn3c(-c4cc5ccncc5s4)cnc3c2)cc1] | 114 |
| 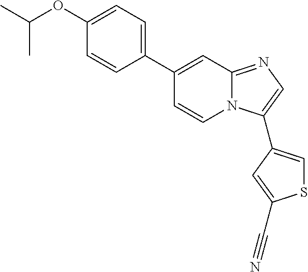 [SMILES: CC(C)Oc1ccc(-c2ccn3c(-c4csc(C#N)c4)cnc3c2)cc1] | 115 |
| 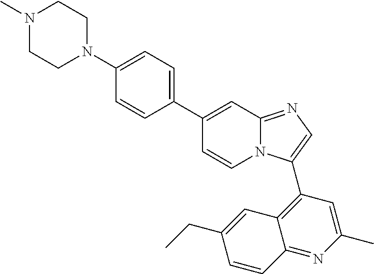 | 116 |
| 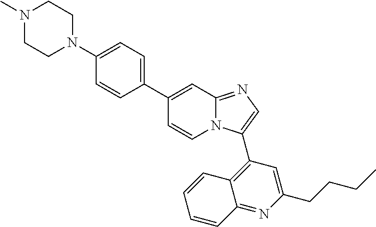 [SMILES: CCCCc1cc(-c2cnc3cc(-c4ccc(N5CCN(C)CC5)cc4)ccn23)c2ccccc2n1] | 117 |
| 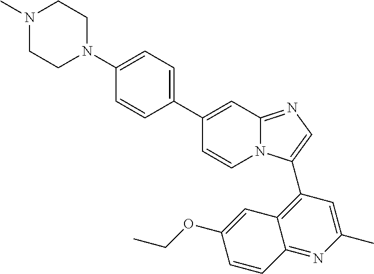 | 118 |
| 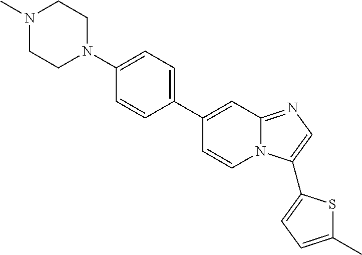 | 119 |
| 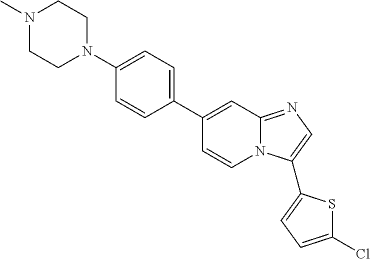 [SMILES: CN1CCN(c2ccc(-c3ccn4c(-c5ccc(Cl)s5)cnc4c3)cc2)CC1] | 120 |
| 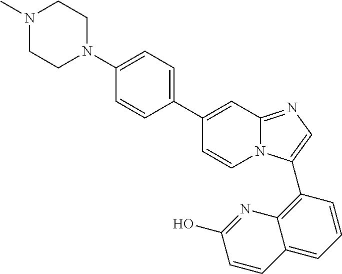 [SMILES: CN1CCN(c2ccc(-c3ccn4c(-c5cccc6ccc(O)nc56)cnc4c3)cc2)CC1] | 121 |
| 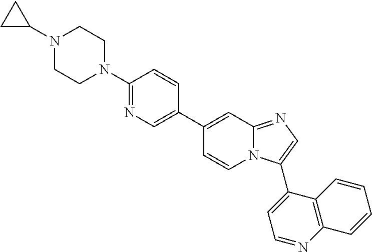 [SMILES: c1ccc2c(-c3cnc4cc(-c5ccc(N6CCN(C7CC7)CC6)nc5)ccn34)ccnc2c1] | 122 |
| 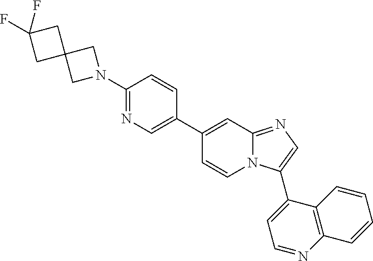 [SMILES: FC1(F)CC2(CN(c3ccc(-c4ccn5c(-c6ccnc7ccccc67)cnc5c4)cn3)C2)C1] | 123 |
| 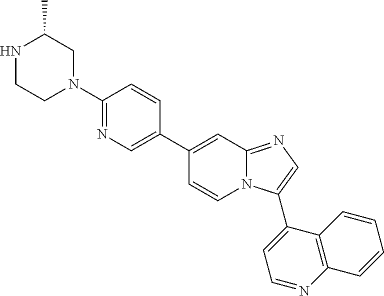 [SMILES: C[C@@H]1CN(c2ccc(-c3ccn4c(-c5ccnc6ccccc56)cnc4c3)cn2)CCN1] | 124 |
| 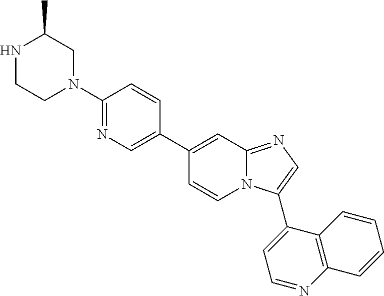 [SMILES: C[C@H]1CN(c2ccc(-c3ccn4c(-c5ccnc6ccccc56)cnc4c3)cn2)CCN1] | 125 |
| 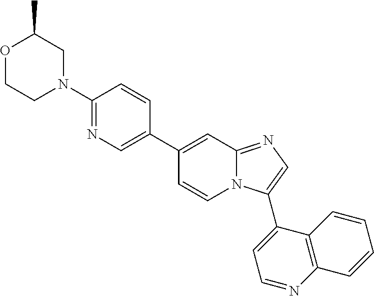 [SMILES: C[C@H]1CN(c2ccc(-c3ccn4c(-c5ccnc6ccccc56)cnc4c3)cn2)CCO1] | 126 |
| 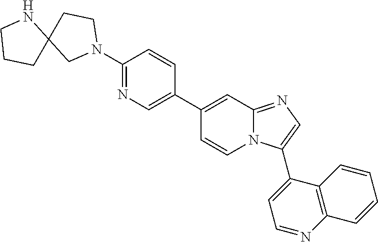 [SMILES: c1ccc2c(-c3cnc4cc(-c5ccc(N6CCC7(CCCN7)C6)nc5)ccn34)ccnc2c1] | 127 |
| 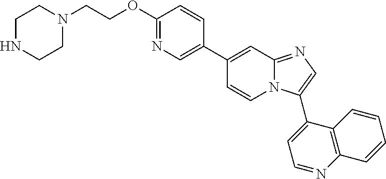 [SMILES: c1ccc2c(-c3cnc4cc(-c5ccc(OCCN6CCNCC6)nc5)ccn34)ccnc2c1] | 128 |
| 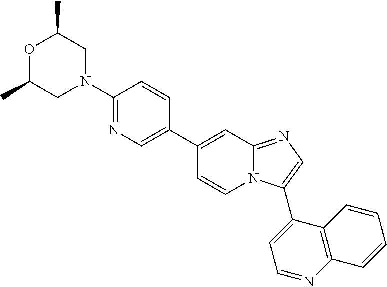 [SMILES: C[C@@H]1CN(c2ccc(-c3ccn4c(-c5ccnc6ccccc56)cnc4c3)cn2)C[C@H](C)O1] | 129 |
| 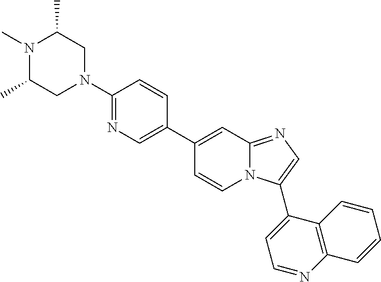 [SMILES: C[C@@H]1CN(c2ccc(-c3ccn4c(-c5ccnc6ccccc56)cnc4c3)cn2)C[C@H](C)N1C] | 130 |
| 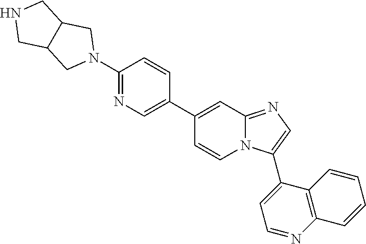 [SMILES: c1ccc2c(-c3cnc4cc(-c5ccc(N6CC7CNCC7C6)nc5)ccn34)ccnc2c1] | 131 |
| 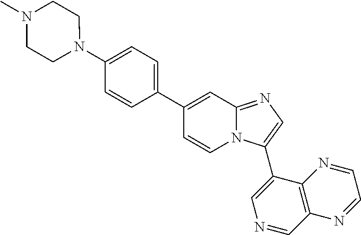 [SMILES: CN1CCN(c2ccc(-c3ccn4c(-c5cncc6nccnc56)cnc4c3)cc2)CC1] | 132 |
| 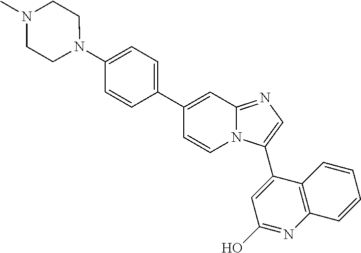 [SMILES: CN1CCN(c2ccc(-c3ccn4c(-c5cc(O)nc6ccccc56)cnc4c3)cc2)CC1] | 133 |
| 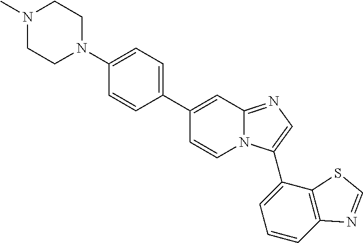 [SMILES: CN1CCN(c2ccc(-c3ccn4c(-c5cccc6ncsc56)cnc4c3)cc2)CC1] | 134 |
| 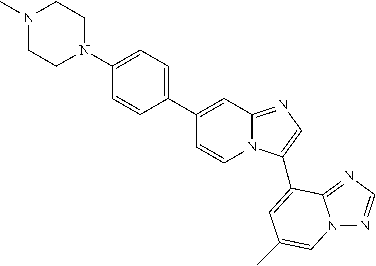 [SMILES: Cc1cc(-c2cnc3cc(-c4ccc(N5CCN(C)CC5)cc4)ccn23)c2ncnn2c1] | 135 |
| 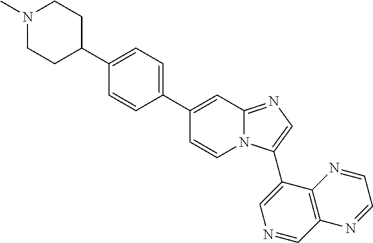 [SMILES: CN1CCC(c2ccc(-c3ccn4c(-c5cncc6nccnc56)cnc4c3)cc2)CC1] | 136 |
| 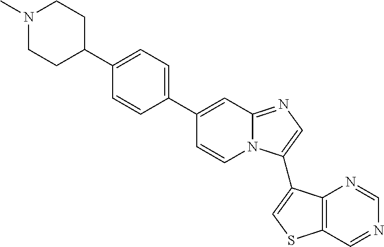 [SMILES: CN1CCC(c2ccc(-c3ccn4c(-c5csc6cncnc56)cnc4c3)cc2)CC1] | 137 |
| 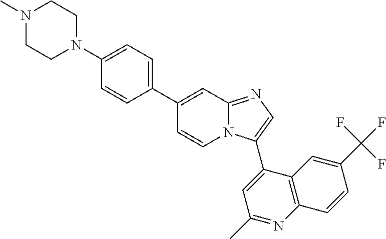 [SMILES: Cc1cc(-c2cnc3cc(-c4ccc(N5CCN(C)CC5)cc4)ccn23)c2cc(C(F)(F)F)ccc2n1] | 138 |
| 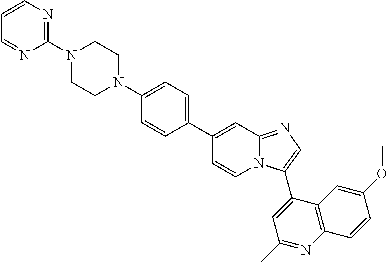 [SMILES: COc1ccc2nc(C)cc(-c3cnc4cc(-c5ccc(N6CCN(c7ncccn7)CC6)cc5)ccn34)c2c1] | 139 |
| 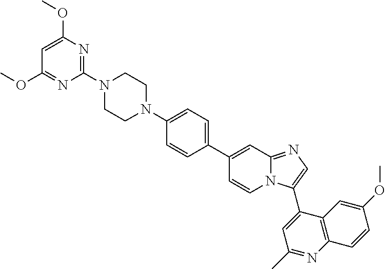 | 140 |
| 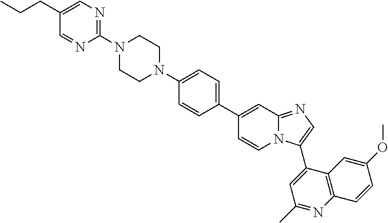 [SMILES: CCCc1cnc(N2CCN(c3ccc(-c4ccn5c(-c6cc(C)nc7ccc(OC)cc67)cnc5c4)cc3)CC2)nc1] | 141 |
| 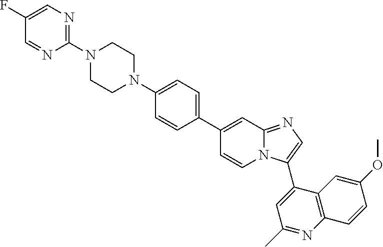 [SMILES: COc1ccc2nc(C)cc(-c3cnc4cc(-c5ccc(N6CCN(c7ncc(F)cn7)CC6)cc5)ccn34)c2c1] | 142 |
| 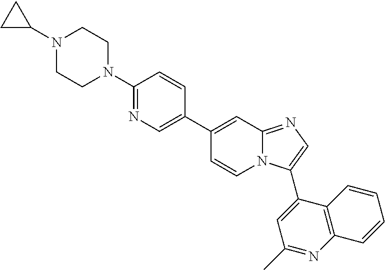 [SMILES: Cc1cc(-c2cnc3cc(-c4ccc(N5CCN(C6CC6)CC5)nc4)ccn23)c2ccccc2n1] | 143 |
| 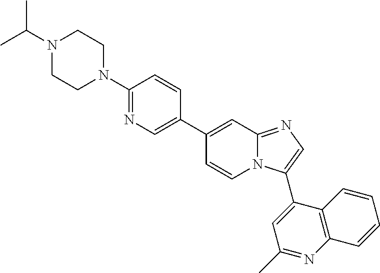 [SMILES: Cc1cc(-c2cnc3cc(-c4ccc(N5CCN(C(C)C)CC5)nc4)ccn23)c2ccccc2n1] | 144 |
| 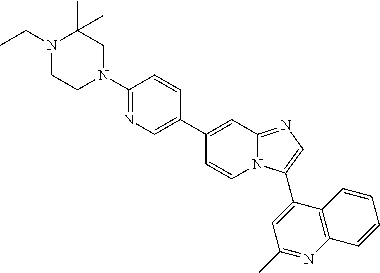 [SMILES: CCN1CCN(c2ccc(-c3ccn4c(-c5cc(C)nc6ccccc56)cnc4c3)cn2)CC1(C)C] | 145 |
| 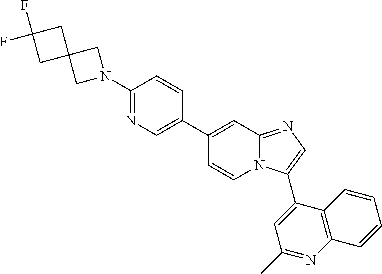 [SMILES: Cc1cc(-c2cnc3cc(-c4ccc(N5CC6(C5)CC(F)(F)C6)nc4)ccn23)c2ccccc2n1] | 146 |
| 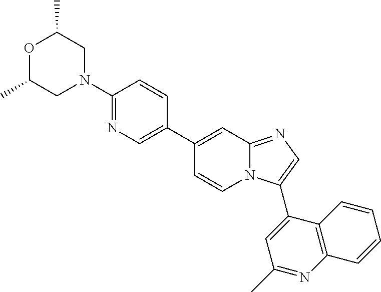 [SMILES: Cc1cc(-c2cnc3cc(-c4ccc(N5C[C@@H](C)O[C@@H](C)C5)nc4)ccn23)c2ccccc2n1] | 147 |
| 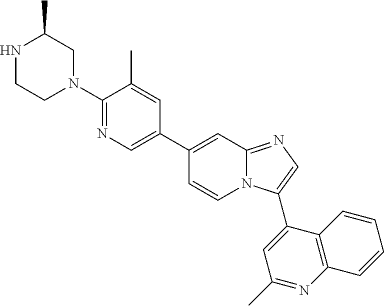 [SMILES: Cc1cc(-c2cnc3cc(-c4cnc(N5CCN[C@@H](C)C5)c(C)c4)ccn23)c2ccccc2n1] | 148 |
| 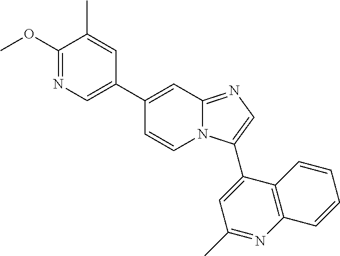 [SMILES: COc1ncc(-c2ccn3c(-c4cc(C)nc5ccccc45)cnc3c2)cc1C] | 149 |
| 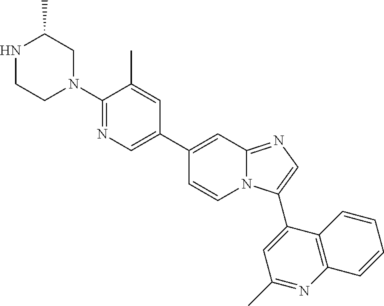 [SMILES: Cc1cc(-c2cnc3cc(-c4cnc(N5CCN[C@H](C)C5)c(C)c4)ccn23)c2ccccc2n1] | 150 |
| 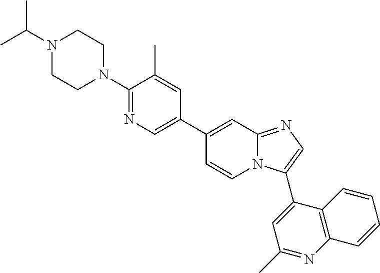 [SMILES: Cc1cc(-c2cnc3cc(-c4cnc(N5CCN(C(C)C)CC5)c(C)c4)ccn23)c2ccccc2n1] | 151 |
| 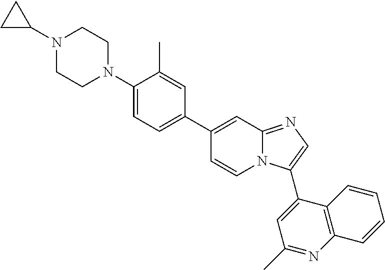 [SMILES: Cc1cc(-c2cnc3cc(-c4ccc(N5CCN(C6CC6)CC5)c(C)c4)ccn23)c2ccccc2n1] | 152 |
| 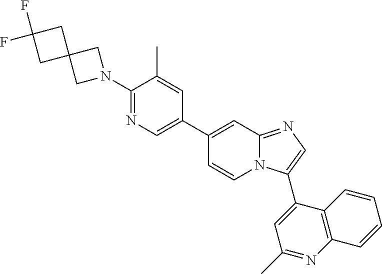 [SMILES: Cc1cc(-c2cnc3cc(-c4cnc(N5CC6(C5)CC(F)(F)C6)c(C)c4)ccn23)c2ccccc2n1] | 153 |
| 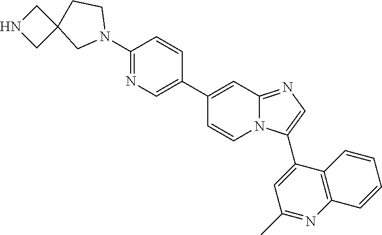 [SMILES: Cc1cc(-c2cnc3cc(-c4ccc(N5CCC6(CNC6)C5)nc4)ccn23)c2ccccc2n1] | 154 |
| 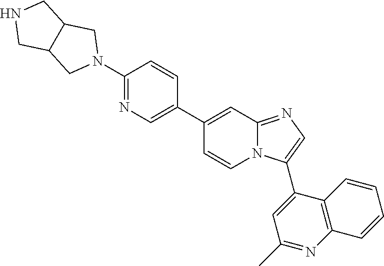 [SMILES: Cc1cc(-c2cnc3cc(-c4ccc(N5CC6CNCC6C5)nc4)ccn23)c2ccccc2n1] | 155 |
| 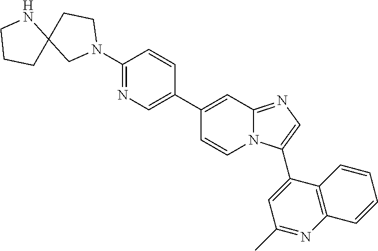 [SMILES: Cc1cc(-c2cnc3cc(-c4ccc(N5CCC6(CCCN6)C5)nc4)ccn23)c2ccccc2n1] | 156 |
| 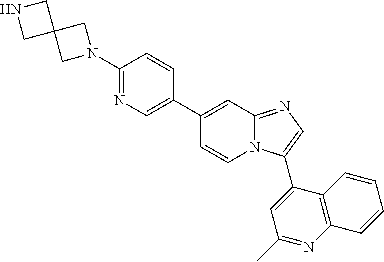 [SMILES: Cc1cc(-c2cnc3cc(-c4ccc(N5CC6(CNC6)C5)nc4)ccn23)c2ccccc2n1] | 157 |
| 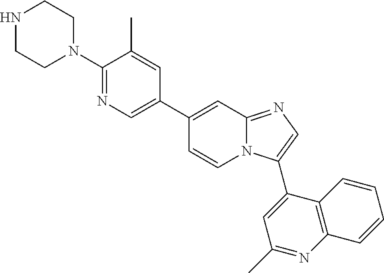 [SMILES: Cc1cc(-c2cnc3cc(-c4cnc(N5CCNCC5)c(C)c4)ccn23)c2ccccc2n1] | 158 |
| 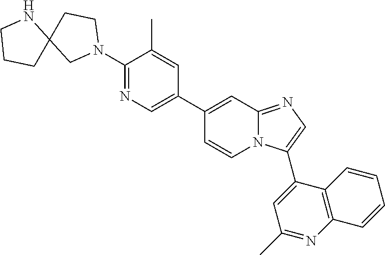 [SMILES: Cc1cc(-c2cnc3cc(-c4cnc(N5CCC6(CCCN6)C5)c(C)c4)ccn23)c2ccccc2n1] | 159 |
| 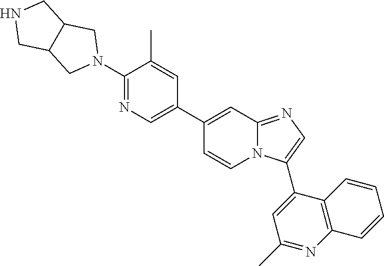 [SMILES: Cc1cc(-c2cnc3cc(-c4cnc(N5CC6CNCC6C5)c(C)c4)ccn23)c2ccccc2n1] | 160 |
| 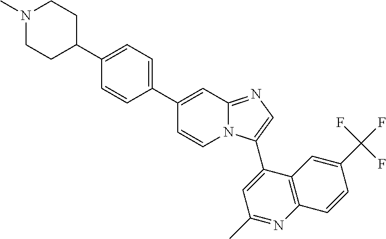 [SMILES: Cc1cc(-c2cnc3cc(-c4ccc(C5CCN(C)CC5)cc4)ccn23)c2cc(C(F)(F)F)ccc2n1] | 161 |
| 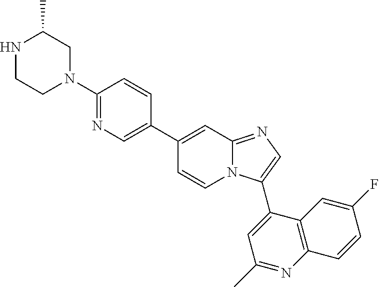 [SMILES: Cc1cc(-c2cnc3cc(-c4ccc(N5CCN[C@H](C)C5)nc4)ccn23)c2cc(F)ccc2n1] | 162 |
| 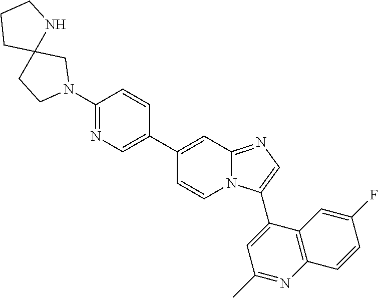 [SMILES: Cc1cc(-c2cnc3cc(-c4ccc(N5CCC6(CCCN6)C5)nc4)ccn23)c2cc(F)ccc2n1] | 163 |
| 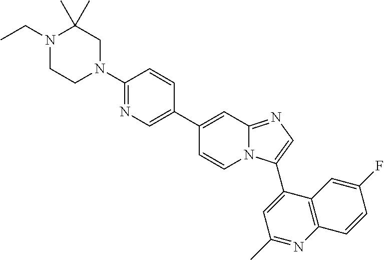 [SMILES: CCN1CCN(c2ccc(-c3ccn4c(-c5cc(C)nc6ccc(F)cc56)cnc4c3)cn2)CC1(C)C] | 164 |
| 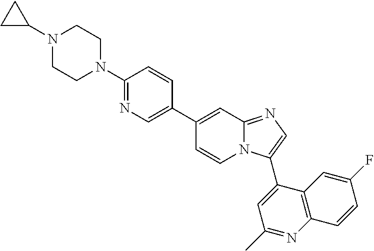 [SMILES: Cc1cc(-c2cnc3cc(-c4ccc(N5CCN(C6CC6)CC5)nc4)ccn23)c2cc(F)ccc2n1] | 165 |
| 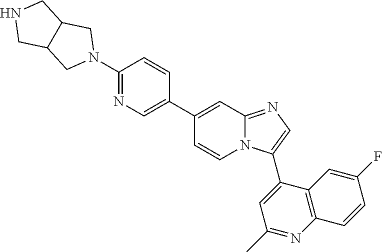 [SMILES: Cc1cc(-c2cnc3cc(-c4ccc(N5CC6CNCC6C5)nc4)ccn23)c2cc(F)ccc2n1] | 166 |
| 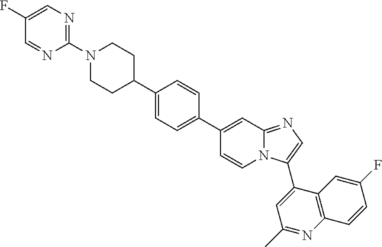 [SMILES: Cc1cc(-c2cnc3cc(-c4ccc(C5CCN(c6ncc(F)cn6)CC5)cc4)ccn23)c2cc(F)ccc2n1] | 167 |
| 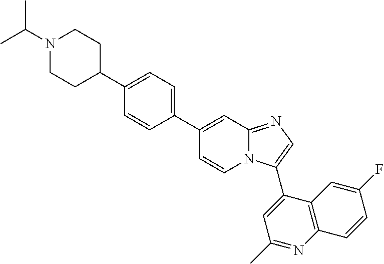 [SMILES: Cc1cc(-c2cnc3cc(-c4ccc(C5CCN(C(C)C)CC5)cc4)ccn23)c2cc(F)ccc2n1] | 168 |
| 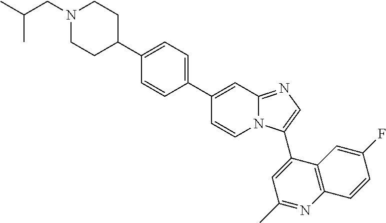 [SMILES: Cc1cc(-c2cnc3cc(-c4ccc(C5CCN(CC(C)C)CC5)cc4)ccn23)c2cc(F)ccc2n1] | 169 |
| 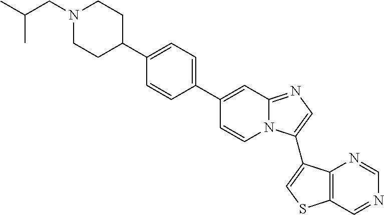 [SMILES: CC(C)CN1CCC(c2ccc(-c3ccn4c(-c5csc6cncnc56)cnc4c3)cc2)CC1] | 170 |
| 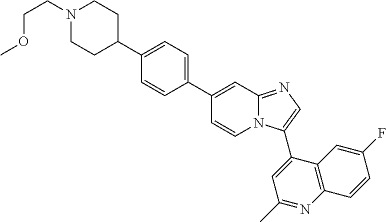 [SMILES: COCCN1CCC(c2ccc(-c3ccn4c(-c5cc(C)nc6ccc(F)cc56)cnc4c3)cc2)CC1] | 171 |
| 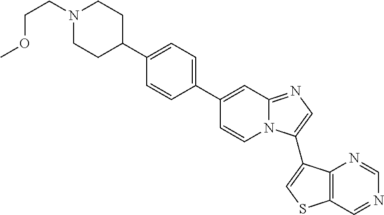 [SMILES: COCCN1CCC(c2ccc(-c3ccn4c(-c5csc6cncnc56)cnc4c3)cc2)CC1] | 172 |
| 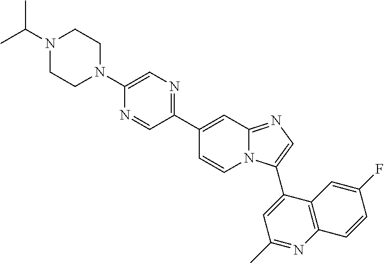 [SMILES: Cc1cc(-c2cnc3cc(-c4cnc(N5CCN(C(C)C)CC5)cn4)ccn23)c2cc(F)ccc2n1] | 173 |
| 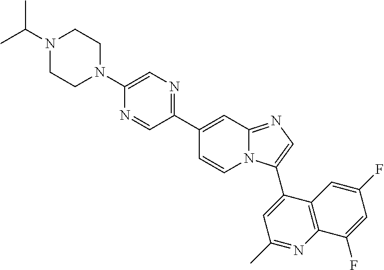 [SMILES: Cc1cc(-c2cnc3cc(-c4cnc(N5CCN(C(C)C)CC5)cn4)ccn23)c2cc(F)cc(F)c2n1] | 174 |
| 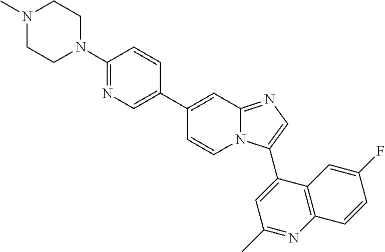 [SMILES: Cc1cc(-c2cnc3cc(-c4ccc(N5CCN(C)CC5)nc4)ccn23)c2cc(F)ccc2n1] | 175 |
| 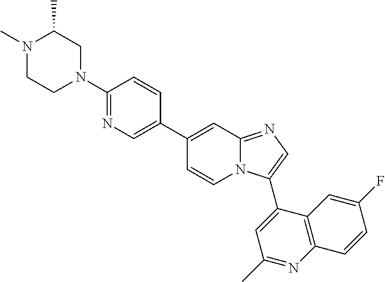 [SMILES: Cc1cc(-c2cnc3cc(-c4ccc(N5CCN(C)[C@H](C)C5)nc4)ccn23)c2cc(F)ccc2n1] | 176 |
| 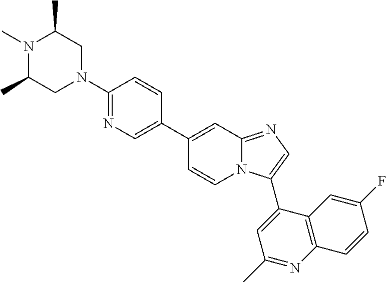 [SMILES: Cc1cc(-c2cnc3cc(-c4ccc(N5C[C@@H](C)N(C)[C@@H](C)C5)nc4)ccn23)c2cc(F)ccc2n1] | 177 |
| 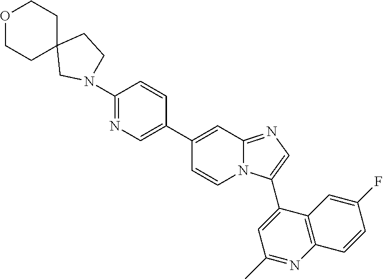 [SMILES: Cc1cc(-c2cnc3cc(-c4ccc(N5CCC6(CCOCC6)C5)nc4)ccn23)c2cc(F)ccc2n1] | 178 |
| 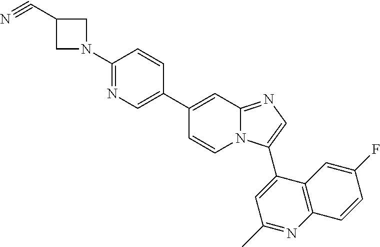 [SMILES: Cc1cc(-c2cnc3cc(-c4ccc(N5CC(C#N)C5)nc4)ccn23)c2cc(F)ccc2n1] | 179 |
| 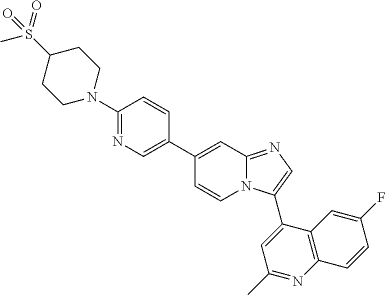 [SMILES: Cc1cc(-c2cnc3cc(-c4ccc(N5CCC(S(C)(=O)=O)CC5)nc4)ccn23)c2cc(F)ccc2n1] | 180 |
| 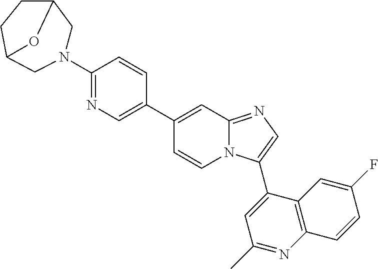 [SMILES: Cc1cc(-c2cnc3cc(-c4ccc(N5CC6CCC(C5)O6)nc4)ccn23)c2cc(F)ccc2n1] | 181 |
| 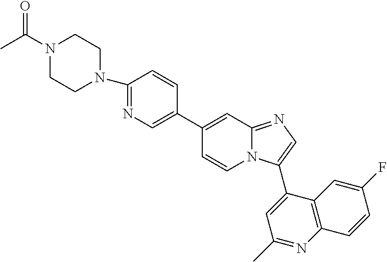 [SMILES: CC(=O)N1CCN(c2ccc(-c3ccn4c(-c5cc(C)nc6ccc(F)cc56)cnc4c3)cn2)CC1] | 182 |
| 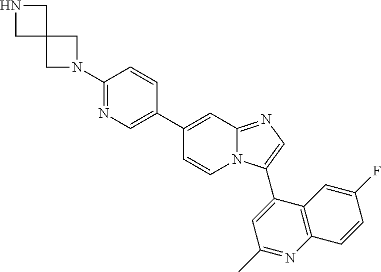 [SMILES: Cc1cc(-c2cnc3cc(-c4ccc(N5CC6(CNC6)C5)nc4)ccn23)c2cc(F)ccc2n1] | 183 |
| 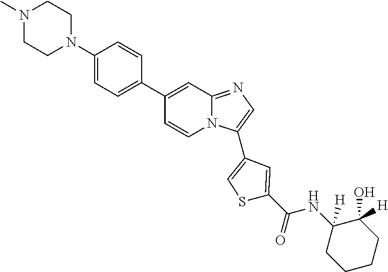 [SMILES: CN1CCN(c2ccc(-c3ccn4c(-c5csc(C(=O)N[C@@H]6CCCC[C@H]6O)c5)cnc4c3)cc2)CC1] | 184 |
| 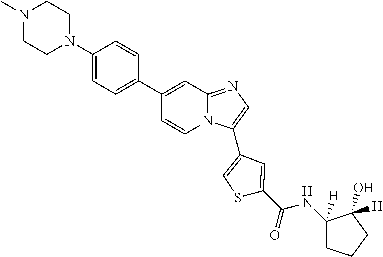 [SMILES: CN1CCN(c2ccc(-c3ccn4c(-c5csc(C(=O)N[C@@H]6CCC[C@H]6O)c5)cnc4c3)cc2)CC1] | 185 |
| 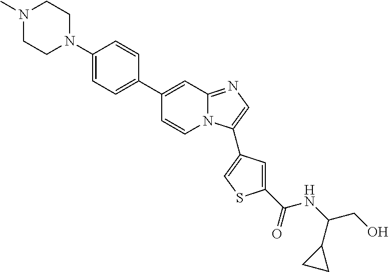 [SMILES: CN1CCN(c2ccc(-c3ccn4c(-c5csc(C(=O)NC(CO)C6CC6)c5)cnc4c3)cc2)CC1] | 186 |
| 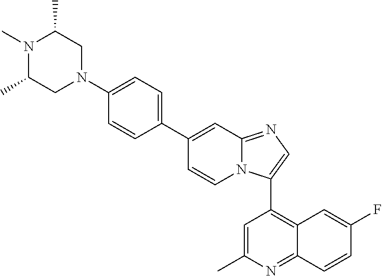 [SMILES: Cc1cc(-c2cnc3cc(-c4ccc(N5C[C@@H](C)N(C)[C@@H](C)C5)cc4)ccn23)c2cc(F)ccc2n1] | 187 |
| 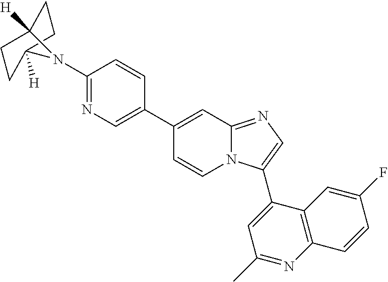 [SMILES: Cc1cc(-c2cnc3cc(-c4ccc(N5[C@H]6CC[C@@H]5CC6)nc4)ccn23)c2cc(F)ccc2n1] | 188 |
| 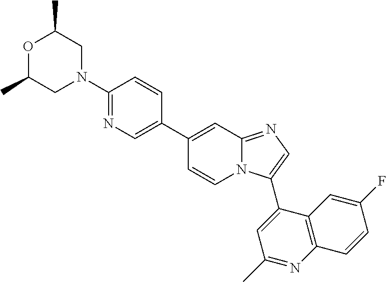 [SMILES: Cc1cc(-c2cnc3cc(-c4ccc(N5C[C@@H](C)O[C@@H](C)C5)nc4)ccn23)c2cc(F)ccc2n1] | 189 |
| 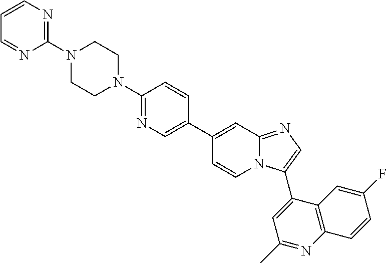 [SMILES: Cc1cc(-c2cnc3cc(-c4ccc(N5CCN(c6ncccn6)CC5)nc4)ccn23)c2cc(F)ccc2n1] | 190 |
| 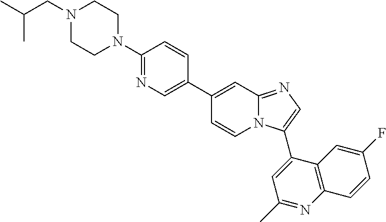 [SMILES: Cc1cc(-c2cnc3cc(-c4ccc(N5CCN(CC(C)C)CC5)nc4)ccn23)c2cc(F)ccc2n1] | 191 |
| 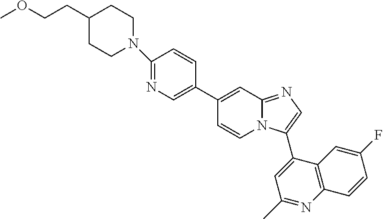 [SMILES: COCCC1CCN(c2ccc(-c3ccn4c(-c5cc(C)nc6ccc(F)cc56)cnc4c3)cn2)CC1] | 192 |
| 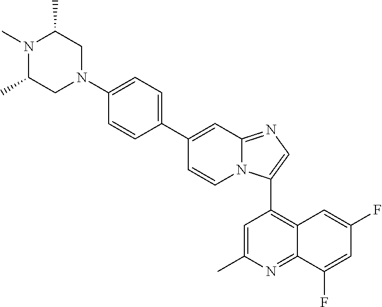 [SMILES: Cc1cc(-c2cnc3cc(-c4ccc(N5C[C@@H](C)N(C)[C@@H](C)C5)cc4)ccn23)c2cc(F)cc(F)c2n1] | 193 |
| 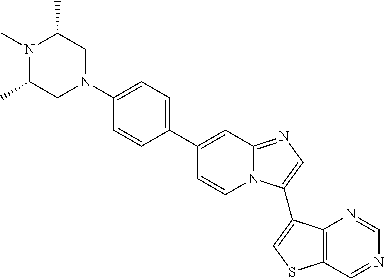 [SMILES: C[C@@H]1CN(c2ccc(-c3ccn4c(-c5csc6cncnc56)cnc4c3)cc2)C[C@H](C)N1C] | 194 |
| 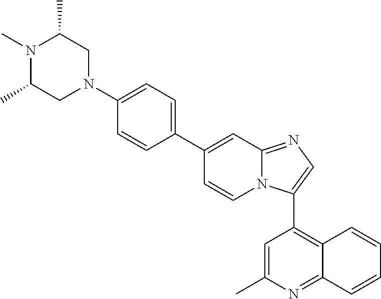 [SMILES: Cc1cc(-c2cnc3cc(-c4ccc(N5C[C@@H](C)N(C)[C@@H](C)C5)cc4)ccn23)c2ccccc2n1] | 195 |
| 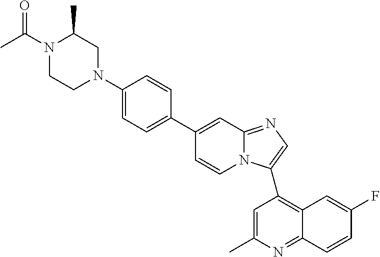 [SMILES: CC(=O)N1CCN(c2ccc(-c3ccn4c(-c5cc(C)nc6ccc(F)cc56)cnc4c3)cc2)C[C@@H]1C] | 196 |
| 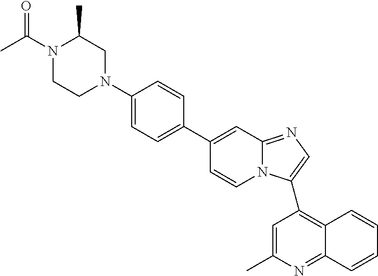 [SMILES: CC(=O)N1CCN(c2ccc(-c3ccn4c(-c5cc(C)nc6ccccc56)cnc4c3)cc2)C[C@@H]1C] | 197 |
| 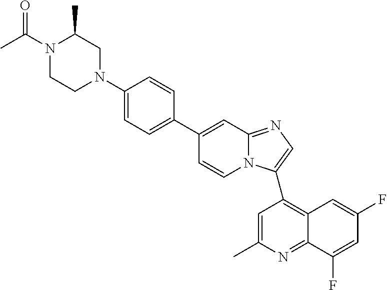 [SMILES: CC(=O)N1CCN(c2ccc(-c3ccn4c(-c5cc(C)nc6c(F)cc(F)cc56)cnc4c3)cc2)C[C@@H]1C] | 198 |
| 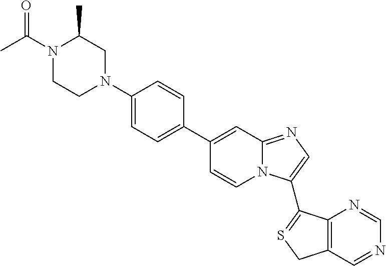 | 199 |
| 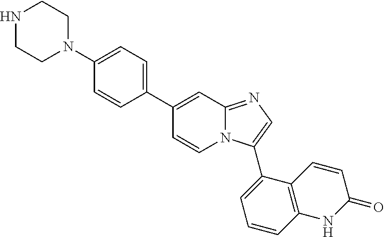 [SMILES: O=c1ccc2c(-c3cnc4cc(-c5ccc(N6CCNCC6)cc5)ccn34)cccc2[nH]1] | 200 |
| 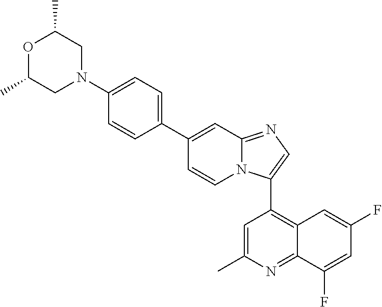 [SMILES: Cc1cc(-c2cnc3cc(-c4ccc(N5C[C@@H](C)O[C@@H](C)C5)cc4)ccn23)c2cc(F)cc(F)c2n1] | 201 |
| 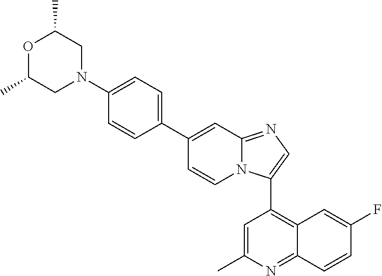 [SMILES: Cc1cc(-c2cnc3cc(-c4ccc(N5C[C@@H](C)O[C@@H](C)C5)cc4)ccn23)c2cc(F)ccc2n1] | 202 |
| 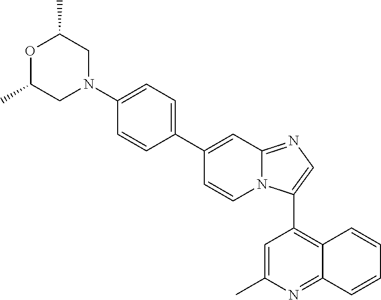 [SMILES: Cc1cc(-c2cnc3cc(-c4ccc(N5C[C@@H](C)O[C@@H](C)C5)cc4)ccn23)c2ccccc2n1] | 203 |
| 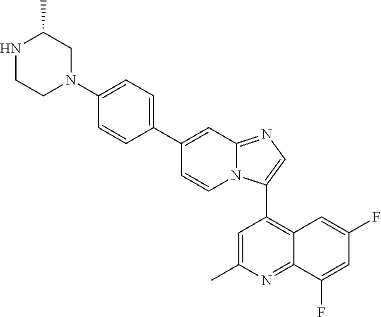 [SMILES: Cc1cc(-c2cnc3cc(-c4ccc(N5CCN[C@H](C)C5)cc4)ccn23)c2cc(F)cc(F)c2n1] | 204 |
| 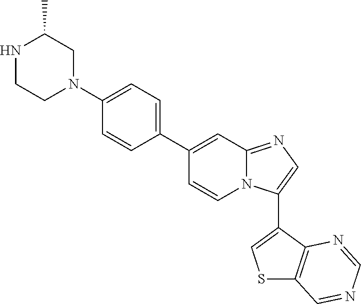 [SMILES: C[C@@H]1CN(c2ccc(-c3ccn4c(-c5csc6cncnc56)cnc4c3)cc2)CCN1] | 205 |

### II. Pharmaceutical Compositions

In certain embodiments, the present invention provides pharmaceutical compositions comprising a compound of Formula I and a pharmaceutically acceptable carrier.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

This invention includes the use of pharmaceutically acceptable salts of compounds of the invention in the compositions and methods of the present invention. The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound of Formula I or Formula II. As another example, the salt may comprise less than one inorganic or organic acid molecule per molecule of base, such as two molecules of compound of Formula I or Formula II per molecule of tartaric acid.

In further embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### III. Uses of Compounds and Compositions

In certain aspects, the invention provides methods of treating or preventing a disease or condition, comprising administering to a subject a compound of Formula I, e.g., in a therapeutically effective amount or a composition comprising a compound of Formula I.

In some embodiments, the disease is cancer. In some embodiments, the cancer is colorectal cancer, juvenile polyposis syndrome, sporadic colorectal cancer, leukemia, acute myeloid leukemia, acute megakaryoblastic leukemia (AMKL), non-Down syndrome AMKL, Down syndrome AMKL, chronic myelogenous leukemia, lung cancer, non-small cell lung cancer (NSCLC), pancreatic cancer, ovarian cancer, serous ovarian cancer, epithelial ovarian cancer, osteosarcomas, prostate cancer, bone cancer, renal cell cancer, breast cancer, melanoma, or head and neck squamous cell carcinoma (HNSCC).

In some embodiments, the cancer is a cancer of the central nervous system. In some embodiments, the cancer is a glioma, astrocytic glioma, diffuse intrinsic pontine glioma (DIPG), high grade glioma (HGG), germ cell tumor, glioblastoma multiform (GBM), oligodendroglioma, pituitary tumor, or ependymoma.

In certain embodiments, the cancer is a solid tumor. The subject is generally one who has been diagnosed as having a cancerous tumor or one who has been previously treated for a cancerous tumor (e.g., where the tumor has been previously removed by surgery). The cancerous tumor may be a primary tumor and/or a secondary (e.g., metastatic) tumor.

In certain embodiments, the subject is a mammal, e.g., a human.

In some embodiments, the disease is anemia, iron-refractory iron-deficient anemia (IRIDA), iron deficiency anemia, anemia of chronic disease, heterotopic ossification, nonhereditary myositis ossificans, myositis ossificans traumatica, myositis ossificans circumscripta, fibrodysplasia ossificans progressiva (FOP), inflammation, pathologic bone function, ectopic or maladaptive bone formation, a skin disease, hypertension, ventricular hypertrophy, atherosclerosis, spinal cord injury and neuropathy, heart disease, heart damage, liver damage, or liver disease.

In certain embodiments, the invention provides methods of inhibiting proliferation of a cancerous cell comprising contacting a cancerous cell with an effective amount of a compound of Formula I.

The invention also provides methods of inhibiting proliferation of a cancer cell, comprising contacting a cancer cell with a compound of Formula I or a composition comprising a compound of Formula I.

The invention also provides method for propagating, engrafting, or differentiating a progenitor cell, comprising contacting the cell with a compound of Formula I or a composition comprising a compound of Formula I in an amount effective to propagate, engraft, or differentiate the progenitor cell.

The invention also provides methods of modulating the BMP signaling pathway in a cell, comprising contacting a cell with a compound of Formula I. Such methods may be performed in vivo or in vitro.

### IV. Definitions

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, —OCF₃, ethoxy, propoxy, tert-butoxy and the like.

The term “cycloalkyloxy” refers to a cycloakyl group having an oxygen attached thereto.

The term “alkoxyalkyl” refers to an alkyl group substituted with an alkoxy group and may be represented by the general formula alkyl-O-alkyl.

The term “alkylaminoalkyl” refers to an alkyl group substituted with an alkylamino group.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

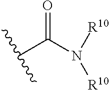

wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

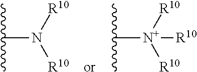

wherein each R¹⁰ independently represents a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

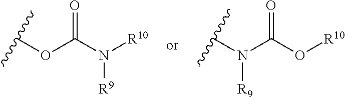

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The term “heteroalkylamino”, as used herein, refers to an amino group substituted with a heteralkyl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, benzimidazole, quinoline, isoquinoline, quinoxaline, quinazoline, indole, isoindole, indazole, benzoxazole, pyrazine, pyridazine, purine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like. Heterocyclyl groups can also be substituted by oxo groups. For example, “heterocyclyl” encompasses both pyrrolidine and pyrrolidinone.

The term “heterocycloalkyl”, as used herein, refers to an alkyl group substituted with a heterocycle group.

The term “heterocycloalkylamino”, as used herein refers to an amino group substituted with a heterocycloalkyl group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

As used herein, the term “oxo” refers to a carbonyl group. When an oxo substituent occurs on an otherwise saturated group, such as with an oxo-substituted cycloalkyl group (e.g., 3-oxo-cyclobutyl), the substituted group is still intended to be a saturated group. When a group is referred to as being substituted by an “oxo” group, this can mean that a carbonyl moiety (i.e., —C(═O)—) replaces a methylene unit (i.e., —CH₂—).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

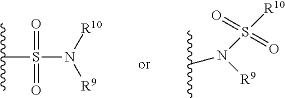

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

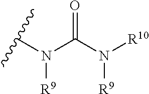

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “prodrug” is intended to encompass compounds which, under physiologic conditions, are converted into the therapeutically active agents of the present invention (e.g., a compound of formula I). A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of formula I in a formulation represented above can be replaced with the corresponding suitable prodrug, e.g., wherein a hydroxyl in the parent compound is presented as an ester or a carbonate or carboxylic acid present in the parent compound is presented as an ester.

### EXAMPLES

Examples of compounds of Formula I or pharmaceutically acceptable salts thereof having useful biological activity are listed above in Table 1. The preparation of these compounds can be realized by one of skilled in the art of organic synthesis using known techniques and methodology.

### Example 1: Chemical Syntheses

The general procedures used in the methods to prepare the compounds of the present invention are described below and are analogous to those described in International Application No. PCT/US2013/032588, incorporated by reference in its entirety, and specifically with respect to the methods of preparing the compounds disclosed therein.

The characterization data of certain compounds of Formula I is summarized in Table 2.

TABLE 2
Characterization Data of Exemplary Compounds of Formula I

| Cmpd | STRUCTURE_NAME | Characterization | MW_EXACT |
|---|---|---|---|
| 1 | 7-(4-isopropoxyphenyl)-3-(thiophen-2- | LCMS: Rt = 0.875 min, M + | 334.11398 |
| | yl)imidazo[1,2-a]pyridine | H = 335.0; >98% @ 215 | |
| | | and 254 nm | |
| 2 | 8-fluoro-4-(7-(4-(4-methylpiperazin-1- | LCMS: Rt = 0.637 min, M + | 437.20157 |
| | yl)phenyl)imidazo[1,2-a]pyridin-3- | H = 438.2; >98% @ 215 | |
| | yl)quinoline | and 254 nm | |
| 3 | 4-(7-(4-(4-methylpiperazin-1- | LCMS: Rt = 0.575 min, M + | 420.20624 |
| | yl)phenyl)imidazo[1,2-a]pyridin-3-yl)- | H = 421.2; >98% @ 215 | |
| | 1,5-naphthyridine | and 254 nm | |
| 4 | 4-[7-[6-(4-isobutylpiperazin-1-yl)-3- | LCMS: Rt = 0.621 min, | 462.2532 |
| | pyridyl]imidazo[1,2-a]pyridin-3- | M = H = 463.4; >98% @ 215 | |
| | yl]quinoline; 2,2,2-trifluoroacetic acid | and 254 nm | |
| 5 | 5-[7-[6-(4-isobutylpiperazin-1-yl)-3- | LCMS: Rt = 0.540 min, | 462.2532 |
| | pyridyl]imidazo[1,2-a]pyridin-3- | M + H = 463.3; >98% @ 254 nm | |
| | yl]quinoline; 2,2,2-trifluoroacetic acid | | |
| 6 | 4-[7-[6-(4-isobutylpiperazin-1-yl)-3- | LCMS: Rt = 0.589 min, | 463.24844 |
| | pyridyl]imidazo[1,2-a]pyridin-3-yl]-1,5- | M + H = 464.4; >98% @ 215 | |
| | naphthyridine; 2,2,2-trifluoroacetic acid | and 254 nm | |
| 7 | 1-[(4-methoxyphenyl)methyl]-4-[5-[3- | LCMS: Rt = 0.749 min, | 540.22736 |
| | (4-quinolyl)imidazo[1,2-a]pyridin-7-yl]- | M + H = 541.3; >96% @ 215 | |
| | 2-pyridyl]piperazin-2-one; 2,2,2- | and >98% @ 254 nm | |
| | trifluoroacetic acid | | |
| 8 | 4-[7-[6-(4-methyl-1-piperidyl)-3- | LCMS: Rt = 0.671 min, | 419.211 |
| | pyridyl]imidazo[1,2-a]pyridin-3- | M + H = 420.4; >98% @ 215 | |
| | yl]quinoline; 2,2,2-trifluoroacetic acid | and 254 nm | |
| 9 | 5-[7-[6-(4-methyl-1-piperidyl)-3- | LCMS: Rt = 0.614 min, | 419.211 |
| | pyridyl]imidazo[1,2-a]pyridin-3- | M + H = 420.4; >98% @ 215 | |
| | yl]quinoline; 2,2,2-trifluoroacetic acid | and 254 nm | |
| 10 | 4-[7-[6-(4-methylpiperazin-1-yl)-3- | LCMS: Rt = 0.541 min, | 420.20624 |
| | pyridyl]imidazo[1,2-a]pyridin-3- | M + H = 421.4; >98% @ 215 | |
| | yl]quinoline; 2,2,2-trifluoroacetic acid | and 254 nm | |
| 11 | 5-[7-[6-(4-methylpiperazin-1-yl)-3- | LCMS: Rt = 0.504 min, | 420.20624 |
| | pyridyl]imidazo[1,2-a]pyridin-3- | M + H = 421.4; >90% @ 215 | |
| | yl]quinoline; 2,2,2-trifluoroacetic acid | and 254 nm | |
| 12 | 4-[7-[6-(4-methylpiperazin-1-yl)-3- | LCMS: Rt = 0.510 min, | 421.20148 |
| | pyridyl]imidazo[1,2-a]pyridin-3-yl]-1,5- | M + H = 422.4; >98% @ 215 | |
| | naphthyridine; 2,2,2-trifluoroacetic acid | and 254 nm | |
| 13 | 8-fluoro-4-[7-[6-(4-methylpiperazin-1- | LCMS: Rt = 0.567 min, | 438.19681 |
| | yl)-3-pyridyl]imidazo[1,2-a]pyridin-3- | M + H = 439.3; >98% @ 215 | |
| | yl]quinoline; 2,2,2-trifluoroacetic acid | and 254 nm | |
| 14 | 4-[5-[3-(4-quinolyl)imidazo[1,2- | LCMS: Rt = 0.518 min, | 420.16986 |
| | a]pyridin-7-yl]-2-pyridyl]piperazin-2- | M + H = 421.4; >98% @ 215 | |
| | one; 2,2,2-trifluoroacetic acid | and 254 nm | |
| 15 | 5-[7-[6-[4-(2-methoxyethyl)piperazin-1- | LCMS: Rt = 0.497 min, | 464.23245 |
| | yl]-3-pyridyl]imidazo[1,2-a]pyridin-3- | M + H = 465.4; >98% @ 215 | |
| | yl]quinoline; 2,2,2-trifluoroacetic acid | and 254 nm | |
| 16 | 4-[7-[6-[4-(2-methoxyethyl)piperazin-1- | LCMS: Rt = 0.542 min, | 464.23245 |
| | yl]-3-pyridyl]imidazo[1,2-a]pyridin-3- | M + H = 465.4; >98% @ 215 | |
| | yl]quinoline; 2,2,2-trifluoroacetic acid | and 254 nm | |
| 17 | 6,8-difluoro-2-methyl-4-[7-(4-piperazin- | LCMS: Rt = 0.689 min, | 455.19214 |
| | 1-ylphenyl)imidazo[1,2-a]pyridin-3- | M + H = 456.2; >98% @ 215 | |
| | yl]quinoline; 2,2,2-trifluoroacetic acid | and 254 nm | |
| 18 | 1-[4-[4-[3-(6,8-difluoro-2-methyl-4- | LCMS: Rt = 0.798 min, | 497.20273 |
| | quinolyl)imidazo[1,2-a]pyridin-7- | M + H = 498.2; >98% @ 215 | |
| | yl]phenyl]piperazin-1-yl]ethanone; | and 254 nm | |
| | 2,2,2-trifluoroacetic acid | | |
| 19 | 6,8-difluoro-4-[7-(4-piperazin-1- | LCMS: Rt = 0.638 min, | 441.17651 |
| | ylphenyl)imidazo[1,2-a]pyridin-3- | M + H = 442.2; >98% @ 215 | |
| | yl]quinoline; 2,2,2-trifluoroacetic acid | and 254 nm | |
| 20 | 6-fluoro-2-methyl-4-[7-(4-piperazin-1- | LCMS: Rt = 0.602 min, | 437.20157 |
| | ylphenyl)imidazo[1,2-a]pyridin-3- | M + H = 438.2; >98% @ 215 | |
| | yl]quinoline; 2,2,2-trifluoroacetic acid | and 254 nm | |
| 21 | 6-fluoro-4-[7-(4-piperazin-1- | LCMS: Rt = 0.608 min, | 423.18591 |
| | ylphenyl)imidazo[1,2-a]pyridin-3- | M + H = 424.2; >98% @ 215 | |
| | yl]quinoline; 2,2,2-trifluoroacetic acid | and 254 nm | |
| 22 | 1-[4-[4-[3-(6-fluoro-4- | LCMS: Rt = 0.737 min, | 465.19647 |
| | quinolyl)imidazo[1,2-a]pyridin-7- | M + H = 466.2; >98% @ 215 | |
| | yl]phenyl]piperazin-1-yl]ethanone; | and 254 nm | |
| | 2,2,2-trifluoroacetic acid | | |
| 23 | 8-fluoro-4-[7-(4-piperazin-1- | LCMS: Rt = 0.609 min, | 423.18591 |
| | ylphenyl)imidazo[1,2-a]pyridin-3- | M + H = 424.2; >98% @ 215 | |
| | yl]quinoline; 2,2,2-trifluoroacetic acid | and 254 nm | |
| 24 | 1-[4-[4-[3-(8-fluoro-4- | LCMS: Rt = 0.740 min, | 465.19647 |
| | quinolyl)imidazo[1,2-a]pyridin-7- | M + H = 466.2; >98% @ 215 | |
| | yl]phenyl]piperazin-1-yl]ethanone; | and 254 nm | |
| | 2,2,2-trifluoroacetic acid | | |
| 25 | 2-methyl-4-[7-(4-piperazin-1- | LCMS: Rt = 0.523 min, | 419.211 |
| | ylphenyl)imidazo[1,2-a]pyridin-3- | M + H = 420.2; >98% @ 215 | |
| | yl]quinoline; 2,2,2-trifluoroacetic acid | and 254 nm | |
| 26 | 1-[4-[4-[3-(2-methyl-4- | LCMS: Rt = 0.637 min, | 461.22156 |
| | quinolyl)imidazo[1,2-a]pyridin-7- | M + H = 462.2; >98% @ 215 | |
| | yl]phenyl]piperazin-1-yl]ethanone; | and 254 nm | |
| | 2,2,2-trifluoroacetic acid | | |
| 27 | 3-[7-(4-piperazin-1- | LCMS: Rt = 0.617 min, | 411.15176 |
| | ylphenyl)imidazo[1,2-a]pyridin-3- | M + H = 412.2; >98% @ 215 | |
| | yl]thieno[2,3-b]pyridine; 2,2,2- | and 254 nm | |
| | trifluoroacetic acid | | |
| 28 | 1-[4-[4-(3-thieno[2,3-b]pyridin-3- | LCMS: Rt = 0.749 min, | 453.16232 |
| | ylimidazo[1,2-a]pyridin-7- | M + H = 454.2; >98% @ 215 | |
| | yl)phenyl]piperazin-1-yl]ethanone; | and 254 nm | |
| | 2,2,2-trifluoroacetic acid | | |
| 29 | 4-[7-(4-piperazin-1- | LCMS: Rt = 0.555 min, | 406.19058 |
| | ylphenyl)imidazo[1,2-a]pyridin-3-yl]- | M + H = 407.2; >98% @ 254 | |
| | 1,5-naphthyridine; 2,2,2-trifluoroacetic | nm | |
| | acid | | |
| 30 | 5-[7-(4-piperazin-1- | LCMS: Rt = 0.498 min, | 405.19534 |
| | ylphenyl)imidazo[1,2-a]pyridin-3- | M + H = 406.2; >98% @ 215 | |
| | yl]quinoline; 2,2,2-trifluoroacetic acid | and 254 nm | |
| 31 | 1-[4-[4-[3-(5-quinolyl)imidazo[1,2- | LCMS: Rt = 0.623 min, | 447.2059 |
| | a]pyridin-7-yl]phenyl]piperazin-1- | M + H = 448.2; >98% @ 215 | |
| | yl]ethanone; 2,2,2-trifluoroacetic acid | and 254 nm | |
| 32 | 4-[7-(4-piperazin-1- | LCMS: Rt = 0.553 min, | 405.19534 |
| | ylphenyl)imidazo[1,2-a]pyridin-3- | M + H = 406.2; >98% @ 215 | |
| | yl]quinoline; 2,2,2-trifluoroacetic acid | and 254 nm | |
| 33 | 1-[4-[4-[3-(4-quinolyl)imidazo[1,2- | LCMS: Rt = 0.689 min, | 447.2059 |
| | a]pyridin-7-yl]phenyl]piperazin-1- | M + H = 448.2; >98% @ 215 | |
| | yl]ethanone; 2,2,2-trifluoroacetic acid | and 254 nm | |
| 34 | 4-[7-(4-piperazin-1- | LCMS: Rt = 0.557 min, | 406.19058 |
| | ylphenyl)imidazo[1,2-a]pyridin-3- | M + H = 407.2; >98% @ 215 | |
| | yl]cinnoline; 2,2,2-trifluoroacetic acid | and 254 nm | |
| 35 | 1-[4-[4-(3-cinnolin-4-ylimidazo[1,2- | LCMS: Rt = 0.685 min, | 448.20117 |
| | a]pyridin-7-yl)phenyl]piperazin-1- | M + H = 449.2; >98 @ 215 | |
| | yl]ethanone; 2,2,2-trifluoroacetic acid | and 254 nm | |
| 36 | 7-fluoro-2-methyl-4-[7-(4-piperazin-1- | LCMS: Rt = 0.588 min, | 437.20157 |
| | ylphenyl)imidazo[1,2-a]pyridin-3- | M + H = 438.2; >98% @ 215 | |
| | yl]quinoline; 2,2,2-trifluoroacetic acid | and 254 nM | |
| 37 | 1-[4-[4-[3-(7-fluoro-2-methyl-4- | LCMS: Rt = 0.713 min, | 479.21213 |
| | quinolyl)imidazo[1,2-a]pyridin-7- | M + H = 480.2; >98% @ 215 | |
| | yl]phenyl]piperazin-1-yl]ethanone; | and 254 nm | |
| | 2,2,2-trifluoroacetic acid | | |
| 38 | 3-fluoro-5-[7-(4-piperazin-1- | LCMS: Rt = 0.638 min, | 423.18591 |
| | ylphenyl)imidazo[1,2-a]pyridin-3- | M + H = 424.2; >98% @ 214 | |
| | yl]quinoline; 2,2,2-trifluoroacetic acid | and 254 nm | |
| 39 | 1-[4-[4-[3-(3-fluoro-5- | LCMS: Rt = 0.770 min, | 465.19647 |
| | quinolyl)imidazo[1,2-a]pyridin-7- | M + H = 466.2; >98% @ 215 | |
| | yl]phenyl]piperazin-1-yl]ethanone; | and 254 nm | |
| | 2,2,2-trifluoroacetic acid | | |
| 40 | 2-methyl-4-[7-(4-piperazin-1- | LCMS: Rt = 0.504 min, | 420.20624 |
| | ylphenyl)imidazo[1,2-a]pyridin-3-yl]- | M + H = 421.2; >98% @ 215 | |
| | 1,7-naphthyridine; 2,2,2-trifluoroacetic | and 254 nm | |
| | acid | | |
| 41 | 1-[4-[4-[3-(2-methyl-1,7-naphthyridin- | LCMS: Rt = 0.629 min, | 462.2168 |
| | 4-yl)imidazo[1,2-a]pyridin-7- | M + H = 463.2; >98% @ 215 | |
| | yl]phenyl]piperazin-1-yl]ethanone; | and 254 nm | |
| | 2,2,2-trifluoroacetic acid | | |
| 42 | 3-[7-(4-piperazin-1- | LCMS: Rt = 0.654 min, | 411.15176 |
| | ylphenyl)imidazo[1,2-a]pyridin-3- | M + H = 412.2; >98% @ 254 nm | |
| | yl]thieno[3,2-b]pyridine; 2,2,2- | | |
| | trifluoroacetic acid | | |
| 43 | 1-[4-[4-(3-thieno[3,2-b]pyridin-3- | LCMS: Rt = 0.764 min, | 453.16232 |
| | ylimidazo[1,2-a]pyridin-7- | M + H = 454.2; >98% @ 215 | |
| | yl]phenyl]piperazin-1-yl]ethanone; | and 254 nm | |
| | 2,2,2-trifluoroacetic acid | | |
| 44 | 3-[7-(4-piperazin-1- | LCMS: Rt = 0.626 min, | 395.17462 |
| | ylphenyl)imidazo[1,2-a]pyridin-3- | M + H = 396.2; >98% @ 215 | |
| | yl]furo[3,2-b]pyridine; 2,2,2- | and 254 nm | |
| | trifluoroacetic acid | | |
| 45 | 3-[7-(4-piperazin-1- | LCMS: Rt = 0.444 min, | 411.15176 |
| | ylphenyl)imidazo[1,2-a]pyridin-3- | M + H = 412.2; >98% @ 215 | |
| | yl]thieno[2,3-c]pyridine; 2,2,2- | and 254 nm | |
| | trifluoroacetic acid | | |
| 46 | 1-[4-[4-(3-thieno[2,3-c]pyridin-3- | LCMS: Rt = 0.572 min, | 453.16232 |
| | ylimidazo[1,2-a]pyridin-7- | M + H = 454.2; >98% @ 215 | |
| | yl)phenyl]piperazin-1-yl]ethanone; | and 254 nm | |
| | 2,2,2-trifluoroacetic acid | | |
| 47 | 6-methoxy-2-methyl-4-[7-(4-piperazin- | LCMS: Rt = 0.578 min, | 449.22156 |
| | 1-ylphenyl)imidazo[1,2-a]pyridin-3- | M + H = 450.2; >98% @ 215 | |
| | yl]quinoline; 2,2,2-trifluoroacetic acid | and 254 nm | |
| 48 | 1-[4-[4-[3-(6-methoxy-2-methyl-4- | LCMS: Rt = 0.665 min, | 491.23212 |
| | quinolyl)imidazo[1,2-a]pyridin-7- | M + H = 492.2; >92% @ 215 nm | |
| | yl]phenyl]piperazin-1-yl]ethanone; | | |
| | 2,2,2-trifluoroacetic acid | | |
| 49 | 4-[7-[4-(1-methyl-4- | LCMS: Rt = 0.602 min, | 419.211 |
| | piperidyl)phenyl]imidazo[1,2-a]pyridin- | M + H = 420.2; >98% @ 215 | |
| | 3-yl]-1,5-naphthyridine; 2,2,2- | and 254 nm | |
| | trifluoroacetic acid | | |
| 50 | 1-[4-[4-[3-(2-methyl-5- | LCMS: Rt = 0.584 min, | 461.22156 |
| | quinolyl)imidazo[1,2-a]pyridin-7- | M + H = 462.2; >98% @ 215 | |
| | yl]phenyl]piperazin-1-yl]ethanone; | and 254 nm | |
| | 2,2,2-trifluoroacetic acid | | |
| 51 | 1-[4-[4-[3-(2,6-dimethyl-4- | LCMS: Rt = 0.665 min, | 475.23721 |
| | quinolyl)imidazo[1,2-a]pyridin-7- | M + H = 476.2; >98% @ 215 | |
| | yl]phenyl]piperazin-1-yl]ethanone; | and 254 nm | |
| | 2,2,2-trifluoroacetic acid | | |
| 52 | 1-[4-[4-[3-(7-chloro-8-methyl-4- | LCMS: Rt = 0.927 min, | 495.18259 |
| | quinolyl)imidazo[1,2-a]pyridin-7- | M + H = 496.2; >98% @ 215 | |
| | yl]phenyl]piperazin-1-yl]ethanone; | and 254 nm | |
| | 2,2,2-trifluoroacetic acid | | |
| 53 | 5-[7-[4-(1-methyl-4- | LCMS: Rt = 0.524 min, | 418.21576 |
| | piperidyl)phenyl]imidazo[1,2-a]pyridin- | M + H = 419.2; >98% @ 215 | |
| | 3-yl]quinoline; 2,2,2-trifluoroacetic acid | and 254 nm | |
| 54 | 6,8-difluoro-2-methyl-4-[7-[4-(1- | LCMS: Rt = 0.688 min, | 468.21256 |
| | methyl-4-piperidyl)phenyl]imidazo[1,2- | M + H = 469.2; >98% @ 215 | |
| | a]pyridin-3-yl]quinoline; 2,2,2- | and 254 nm | |
| | trifluoroacetic acid | | |
| 55 | 1-[4-[4-(3-thieno[3,2-d]pyrimidin-7- | LCMS: Rt = 0.674 min, | 454.15759 |
| | ylimidazo[1,2-a]pyridin-7- | M + H = 455.2; >98% @ 215 | |
| | yl)phenyl]piperazin-1-yl]ethanone; | and 254 nm | |
| | 2,2,2-trifluoroacetic acid | | |
| 56 | 1-[4-[4-[3-(1,6-naphthyridin-4- | LCMS: Rt = 0.620 min, | 448.20117 |
| | yl)imidazo[1,2-a]pyridin-7- | M + H = 449.2; >98% @ 215 | |
| | yl]phenyl]piperazin-1-yl]ethanone; | and 254 nm | |
| | 2,2,2-trifluoroacetic acid | | |
| 57 | 2,8-dimethyl-4-[7-(4-piperazin-1- | LCMS: Rt = 0.636 min, | 433.22665 |
| | ylphenyl)imidazo[1,2-a]pyridin-3- | M + H = 434.2; >98% @ 215 | |
| | yl]quinoline; 2,2,2-trifluoroacetic acid | and 254 nm | |
| 58 | 1-[4-[4-[3-(2,8-dimethyl-4- | LCMS: Rt = 0.774 min, | 475.23721 |
| | quinolyl)imidazo[1,2-a]pyridin-7- | M + H = 476.2; >98% @ 215 | |
| | yl]phenyl]piperazin-1-yl]ethanone; | and 254 nm | |
| | 2,2,2-trifluoroacetic acid | | |
| 59 | 7-chloro-8-methyl-4-[7-(4-piperazin-1- | LCMS: Rt = 0.787 min, | 453.17203 |
| | ylphenyl)imidazo[1,2-a]pyridin-3- | M + H = 454.2; >98% @ 215 | |
| | yl]quinoline; 2,2,2-trifluoroacetic acid | and 254 nm | |
| 60 | 2,6-dimethyl-4-[7-(4-piperazin-1- | LCMS: Rt = 0.553 min, | 433.22665 |
| | ylphenyl)imidazo[1,2-a]pyridin-3- | M + H = 435.2; >98% @ 215 | |
| | yl]quinoline; 2,2,2-trifluoroacetic acid | and 254 nM | |
| 61 | 7-[7-(4-piperazin-1- | LCMS: Rt = 0.597 min, | 412.147 |
| | ylphenyl)imidazo[1,2-a]pyridin-3- | M + H = 413.2; >98% @ 215 | |
| | yl]thieno[3,2-d]pyrimidine; 2,2,2- | and 254 nm | |
| | trifluoroacetic acid | | |
| 62 | 2-methyl-5-[7-(4-piperazin-1- | LCMS: Rt = 0.477 min, | 419.211 |
| | ylphenyl)imidazo[1,2-a]pyridin-3- | M + H = 420.2; >98% @ 215 | |
| | yl]quinoline; 2,2,2-trifluoroacetic acid | and 254 nm | |
| 63 | 2,7-dimethyl-4-[7-(4-piperazin-1- | LCMS: Rt = 0.571 min, | 433.22665 |
| | ylphenyl)imidazo[1,2-a]pyridin-3- | M + H = 434.2; >98% @ 215 | |
| | yl]quinoline; 2,2,2-trifluoroacetic acid | and 254 nm | |
| 64 | 6-fluoro-2-methyl-4-[7-[6-[(1-methyl-4- | LCMS: Rt = 0.626 min, | 467.21213 |
| | piperidyl)oxy]-3-pyridyl]imidazo[1,2- | M + H = 468.2; >98% @ 215 | |
| | a]pyridin-3-yl]quinoline | and 254 nm | |
| 65 | 6,8-difluoro-4-[7-[4-(4- | LCMS: Rt = 0.762 min, | 497.23911 |
| | isopropylpiperazin-1- | M + H = 498.2: >98% @ 215 | |
| | yl)phenyl]imidazo[1,2-a]pyridin-3-yl]- | and 254 nm | |
| | 2-methyl-quinoline; 2,2,2-trifluoroacetic | | |
| | acid | | |
| 66 | 6-fluoro-4-[7-[4-(4-isopropylpiperazin- | LCMS: Rt = 0.673 min, | 479.24854 |
| | 1-yl)phenyl]imidazo[1,2-a]pyridin-3- | M + H = 480.2; >98% @ 215 | |
| | yl]-2-methyl-quinoline; 2,2,2- | and 254 nm | |
| | trifluoroacetic acid | | |
| 67 | 4-[7-[4-(4-isopropylpiperazin-1- | LCMS: Rt = 0.636 min, | 447.24228 |
| | yl)phenyl]imidazo[1,2-a]pyridin-3- | M + H = 448.2; >98% @ 215 | |
| | yl]quinoline | and 254 nm | |
| 68 | 6,8-difluoro-2-methyl-4-[7-[6-[(1- | LCMS: Rt = 0.701 min, | 485.20273 |
| | methyl-4-piperidyl)oxy]-3- | M + H = 486.2; >98% @ 215 | |
| | pyridyl]imidazo[1,2-a]pyridin-3- | and 254 nm | |
| | yl]quinoline | | |
| 69 | 4-[7-(2-ethoxypyrimidin-5- | LCMS: Rt = 0.688 min, | 367.14331 |
| | yl)imidazo[1,2-a]pyridin-3-yl]quinoline | M + H = 368.2; >98% @ 215 | |
| | | and 254 nm | |
| 70 | 6-fluoro-2-methyl-4-[7-[6-[2-(1- | LCMS: Rt = 0.645 min, | 481.22778 |
| | piperidyl)ethoxy]-3- | M + H = 482.2; >98% @ 215 | |
| | pyridyl]imidazo[1,2-a]pyridin-3- | and 254 nm | |
| | yl]quinoline | | |
| 71 | 6,8-difluoro-2-methyl-4-[7-[6-[2-(1- | LCMS: Rt = 0.728 min, | 499.21835 |
| | piperidyl)ethoxy]-3- | M + H = 500.2; >98% @ 215 | |
| | pyridyl]imidazo[1,2-a]pyridin-3- | and 254 nm | |
| | yl]quinoline | | |
| 72 | 4-[7-(2-ethoxypyrimidin-5- | LCMS: Rt = 0.648 min, | 381.15897 |
| | yl)imidazo[1,2-a]pyridin-3-yl]-2- | M + H = 382.2; >98% @ 215 | |
| | methyl-quinoline | and 254 nm | |
| 73 | 5-[7-(2-ethoxypyrimidin-5- | LCMS: Rt = 0.589 min, | 367.14331 |
| | yl)imidazo[1,2-a]pyridin-3-yl]quinoline | M + H = 368.2; >90% @ 215 | |
| | | and >98% @ 254 nm | |
| 74 | 4-[7-(2-ethoxypyrimidin-5- | LCMS: Rt = 0.653 min, | 368.13855 |
| | yl)imidazo[1,2-a]pyridin-3-yl]-1,5- | M + H = 369.2; >98% @ 215 | |
| | naphthyridine | and 254 nm | |
| 75 | 4-[7-(2-ethoxypyrimidin-5- | LCMS: Rt = 0.714 min, | 399.14954 |
| | yl)imidazo[1,2-a]pyridin-3-yl]-7-fluoro- | M + H = 400.2; >98% @ 215 | |
| | 2-methyl-quinoline | and 254 nm | |
| 76 | 4-[7-(2-ethoxypyrimidin-5- | LCMS: Rt = 0.614 min, | 382.15421 |
| | yl)imidazo[1,2-a]pyridin-3-yl]-2- | M + H = 383.2; >98% @ 215 | |
| | methyl-1,7-naphthyridine | and 254 nm | |
| 77 | 4-[7-(2-ethoxypyrimidin-5- | LCMS: Rt = 0.715 min, | 399.14954 |
| | yl)imidazo[1,2-a]pyridin-3-yl]-6-fluoro- | M + H = 400.2; >98% @ 215 | |
| | 2-methyl-quinoline | and 254 nm | |
| 78 | 4-[7-(2-ethoxypyrimidin-5- | LCMS: Rt = 0.699 min, | 395.17462 |
| | yl)imidazo[1,2-a]pyridin-3-yl]-2,6- | M + H = 396.2; >98% @ 215 | |
| | dimethyl-quinoline | and 254 nm | |
| 79 | 4-[7-(2-ethoxypyrimidin-5- | LCMS: Rt = 0.661 min, | 368.13855 |
| | yl)imidazo[1,2-a]pyridin-3-yl]cinnoline | M + H = 369.2; >98% @ 215 | |
| | | and 254 nm | |
| 80 | 4-[7-(2-ethoxypyrimidin-5- | LCMS: Rt = 0.708 min, | 395.17462 |
| | yl)imidazo[1,2-a]pyridin-3-yl]-2,7- | M + H = 396.2; >98% @ 215 | |
| | dimethyl-quinoline | and 254 nm | |
| 81 | 2-methyl-4-[7-[6-[(2R)-2-(pyrrolidin-1- | LCMS: Rt = 0.591 min, | 488.26883 |
| | ylmethyl)pyrrolidin-1-yl]-3- | M + H = 489.4; >98% @ 215 | |
| | pyridyl]imidazo[1,2-a]pyridin-3- | and 254 nm | |
| | yl]quinoline | | |
| 82 | 2-methyl-4-[7-[6-[(2R)-2-(pyrrolidin-1- | LCMS: Rt = 0.603 min, | 489.2641 |
| | ylmethyl)pyrrolidin-1-yl]-3- | M + H = 490.4; >98% @ 215 | |
| | pyridyl]imidazo[1,2-a]pyridin-3-yl]-1,7- | and 254 nm | |
| | naphthyridine | | |
| 83 | 4-[7-[6-(3,4,6,7,8,8a-hexahydro-1H- | LCMS: Rt = 0.647 min, | 496.21869 |
| | pyrrolo[1,2-a]pyrazin-2-yl)-3- | M + H = 497.4; >98% @ 215 | |
| | pyridyl]imidazo[1,2-a]pyridin-3-yl]-6,8- | and 254 nm | |
| | difluoro-2-methyl-quinoline | | |
| 84 | 4-[7-[6-(3,4,6,7,8,8a-hexahydro-1H- | LCMS: Rt = 0.625 min, | 478.22812 |
| | pyrrolo[1,2-a]pyrazin-2-yl)-3- | M + H = 479.2; >98% @ 215 | |
| | pyridyl]imidazo[1,2-a]pyridin-3-yl]-6- | and 254 nm | |
| | fluoro-2-methyl-quinoline | | |
| 85 | 4-[7-[6-(3,4,6,7,8,8a-hexahydro-1H- | LCMS: Rt = 0.520 min, | 460.23755 |
| | pyrrolo[1,2-a]pyrazin-2-yl)-3- | M + H = 461.5; >98% @ 215 | |
| | pyridyl]imidazo[1,2-a]pyridin-3-yl]-2- | and 254 nm | |
| | methyl-quinoline | | |
| 86 | 6,8-difluoro-2-methyl-4-[7-[6-(3- | LCMS: Rt = 0.625 min, | 510.23434 |
| | pyrrolidin-1-ylpyrrolidin-1-yl)-3- | M + H = 511.3; >98% @ 215 | |
| | pyridyl]imidazo[1,2-a]pyridin-3- | and 254 nm | |
| | yl]quinoline | | |
| 87 | 6-fluoro-2-methyl-4-[7-[6-(3-pyrrolidin- | LCMS: Rt = 0.540 min, | 492.24377 |
| | 1-ylpyrrolidin-1-yl)-3- | M + H = 493.4; >98% @ 215 | |
| | pyridyl]imidazo[1,2-a]pyridin-3- | and 254 nm | |
| | yl]quinoline | | |
| 88 | 2-methyl-4-[7-[6-(3-pyrrolidin-1- | LCMS: Rt = 0.528 min, | 474.2532 |
| | ylpyrrolidin-1-yl)-3- | M + H = 475.2; >98% @ 215 | |
| | pyridyl]imidazo[1,2-a]pyridin-3- | and 254 nm | |
| | yl]quinoline | | |
| 89 | 4-[4-[3-(2-methyl-4- | LCMS: Rt = 0.709 min, | 420.19501 |
| | quinolyl)imidazo[1,2-a]pyridin-7- | M + H = 421.2; >98% @ 215 | |
| | yl]phenyl]morpholine; 2,2,2- | and 254 nm | |
| | trifluoroacetic acid | | |
| 90 | 4-[4-[3-(6-fluoro-2-methyl-4- | LCMS: Rt = 0.770 min, | 438.18558 |
| | quinolyl)imidazo[1,2-a]pyridin-7- | M + H = 439.2; >98% @ 215 | |
| | yl]phenyl]morpholine; 2,2,2- | and 254 nm | |
| | trifluoroacetic acid | | |
| 91 | 4-[4-[3-(6,8-difluoro-2-methyl-4- | LCMS: Rt = 0.870 min, | 456.17618 |
| | quinolyl)imidazo[1,2-a]pyridin-7- | M + H = 457.2; >98% @ 215 | |
| | yl]phenyl]morpholine; 2,2,2- | and 254 nm | |
| | trifluoroacetic acid | | |
| 92 | 4-[4-[3-(6-methoxy-2-methyl-4- | LCMS: Rt = 0.732 min, | 450.20557 |
| | quinolyl)imidazo[1,2-a]pyridin-7- | M + H = 451.2; >98% @ 215 | |
| | yl]phenyl]morpholine; 2,2,2- | and 254 nm | |
| | trifluoroacetic acid | | |
| 93 | 4-[4-[3-(2-methyl-1,7-naphthyridin-4- | LCMS: Rt = 0.680 min, | 421.19025 |
| | yl)imidazo[1,2-a]pyridin-7- | M + H = 422.2; >98% @ 215 | |
| | yl]phenyl]morpholine; 2,2,2- | and 254 nm | |
| | trifluoroacetic acid | | |
| 94 | 4-[4-[3-(7-fluoro-2-methyl-4- | LCMS: Rt = 0.781 min, | 438.18558 |
| | quinolyl)imidazo[1,2-a]pyridin-7- | M + H = 439.2; >90% @ 215 | |
| | yl]phenyl]morpholine; 2,2,2- | and >98% @ 254 nm | |
| | trifluoroacetic acid | | |
| 95 | 6-methoxy-2-methyl-4-[7-[4-(1-methyl- | LCMS: Rt = 0.599 min, | 462.24197 |
| | 4-piperidyl)phenyl]imidazo[1,2- | M + H = 463.2; >98% @ 215 | |
| | a]pyridin-3-yl]quinoline; 2,2,2- | and 254 nm | |
| | trifluoroacetic acid | | |
| 96 | 4-[7-(4-isopropoxyphenyl)imidazo[1,2- | LCMS: Rt = 0.853 min, | 423.19467 |
| | a]pyridin-3-yl]-6-methoxy-2-methyl- | M + H = 424.2; >98% @ 215 | |
| | quinoline; 2,2,2-trifluoroacetic acid | and 254 nm | |
| 97 | 7-[4-(1-methyl-4-piperidyl)phenyl]-3- | LCMS: Rt = 0.684 min, | 373.16126 |
| | (2-thienyl)imidazo[1,2-a]pyridine; | M + H = 374.2; >98% @ 215 | |
| | 2,2,2-trifluoroacetic acid | and 254 nm | |
| 98 | 7-[4-(1-methyl-4-piperidyl)phenyl]-3- | LCMS: RT = 0.723 min, | 387.17691 |
| | (5-methyl-2-thienyl)imidazo[1,2- | m + H = 388.2, >98% @215 | |
| | a]pyridine; 2,2,2-trifluoroacetic acid | and 254 nm | |
| 99 | 7-[4-(1-methyl-4-piperidyl)phenyl]-3- | LCMS: RT = 0.644 min, | 373.16126 |
| | (3-thienyl)imidazo[1,2-a]pyridine; | m + H = 374.2, >98% @215 | |
| | 2,2,2-trifluoroacetic acid | and 254 nm | |
| 100 | 7-[4-(1-methyl-4-piperidyl)phenyl]-3- | LCMS: RT = 0.682 min, | 387.17691 |
| | (4-methyl-3-thienyl)imidazo[1,2- | m + H = 388.2, >98% @215 | |
| | a]pyridine; 2,2,2-trifluoroacetic acid | and 254 nm | |
| 101 | 2-butyl-4-[7-[4-(1-methyl-4- | LCMS: RT = 0.692 min, | 474.27835 |
| | piperidyl)phenyl]imidazo[1,2-a]pyridin- | m + H = 475.3, >98% @215 | |
| | 3-yl]quinoline; 2,2,2-trifluoroacetic acid | and 254 nm | |
| 102 | 6-ethoxy-2-methyl-4-[7-[4-(1-methyl-4- | LCMS: RT = 0.616 min, | 476.2576 |
| | piperidyl)phenyl]imidazo[1,2-a]pyridin- | m + H = 477.3, >98% @215 | |
| | 3-yl]quinoline; 2,2,2-trifluoroacetic acid | and 254 nm | |
| 103 | 3-(5-chloro-2-thienyl)-7-(4- | LCMS: RT = 1.033 min, | 368.07501 |
| | isopropoxyphenyl)imidazo[1,2- | m + H 369.4, >98% @215 | |
| | a]pyridine; 2,2,2-trifluoroacetic acid | and 254 nm | |
| 104 | 4-[7-(4-isopropoxyphenyl)imidazo[1,2- | LCMS: RT = 0.873 min, | 378.10382 |
| | a]pyridin-3-yl]thiophene-2-carboxylic | m + H = 379.3, >98% @254 | |
| | acid; 2,2,2-trifluoroacetic acid | and TIC | |
| 105 | 7-(4-isopropoxyphenyl)-3-(5-methyl-2- | LCMS: RT = 1.009 min, | 348.12964 |
| | thienyl)imidazo[1,2-a]pyridine; 2,2,2- | m + H = 349.3, >98% @215 | |
| | trifluoroacetic acid | and 254 nm | |
| 106 | 2-butyl-4-[7-(4- | LCMS: RT = 0.933 min, | 435.23105 |
| | isopropoxyphenyl)imidazo[1,2- | m + H = 436.2, >98% @215 | |
| | a]pyridin-3-yl]quinoline; 2,2,2- | and 254 nm | |
| | trifluoroacetic acid | | |
| 107 | 6-ethoxy-4-[7-(4- | LCMS: RT = 0.878 min, | 437.21033 |
| | isopropoxyphenyl)imidazo[1,2- | m + H = 438.3, >98% @215 | |
| | a]pyridin-3-yl]-2-methyl-quinoline; | and 254 nm | |
| | 2,2,2-trifluoroacetic acid | | |
| 108 | 6-ethyl-4-[7-(4- | LCMS: RT = 0.870 min, | 421.21542 |
| | isopropoxyphenyl)imidazo[1,2- | m + H = 422.4, 98% @215 | |
| | a]pyridin-3-yl]-2-methyl-quinoline; | and 254 nm | |
| | 2,2,2-trifluoroacetic acid | | |
| 109 | 7-(4-isopropoxyphenyl)-3-(4-methyl-3- | LCMS: RT = 0.951 min, | 348.12964 |
| | thienyl)imidazo[1,2-a]pyridine; 2,2,2- | m + H = 349.2, 98% @215 | |
| | trifluoroacetic acid | and 254 nm | |
| 110 | 5-[7-[4-(1-methyl-4- | LCMS: RT = 0.504 min, | 419.211 |
| | piperidyl)phenyl]imidazo[1,2-a]pyridin- | m + H = 420.2, >98% @215 | |
| | 3-yl]quinazoline; 2,2,2-trifluoroacetic | and 254 nm | |
| | acid | | |
| 111 | 8-[7-[4-(1-methyl-4- | LCMS: RT = 0.578 min, | 419.211 |
| | piperidyl)phenyl]imidazo[1,2-a]pyridin- | m + H 420.2, >98% @215 | |
| | 3-yl]quinazoline; 2,2,2-trifluoroacetic | and 254 nm | |
| | acid | | |
| 112 | 2-[7-[4-(1-methyl-4- | LCMS: RT = 0.499 min, | 424.17218 |
| | piperidyl)phenyl]imidazo[1,2-a]pyridin- | m + H = 425.2, >98% @215 | |
| | 3-yl]thieno[2,3-c]pyridine; 2,2,2- | and 254 nm | |
| | trifluoroacetic acid | | |
| 113 | 4-[7-[4-(1-methyl-4- | LCMS: RT = 0.619 min, | 398.15653 |
| | piperidyl)phenyl]imidazo[1,2-a]pyridin- | m + H = 399.2, >98% @215 | |
| | 3-yl]thiophene-2-carbonitrile; 2,2,2- | and 254 nm | |
| | trifluoroacetic acid | | |
| 114 | 2-[7-(4-isopropoxyphenyl)imidazo[1,2- | LCMS: RT = 0.746 min, | 385.12488 |
| | a]pyridin-3-yl]thieno[2,3-c]pyridine; | m + H = 386.2, >98% @215 | |
| | 2,2,2-trifluoroacetic acid | and 254 nm | |
| 115 | 4-[7-(4-isopropoxyphenyl)imidazo[1,2- | LCMS: RT = 0.891 min, | 359.10922 |
| | a]pyridin-3-yl]thiophene-2-carbonitrile; | m + H = 360.2, >98% @215 | |
| | 2,2,2-trifluoroacetic acid | and 254 nm | |
| 116 | 6-ethyl-2-methyl-4-[7-[4-(4- | LCMS: RT = 0.588 min, | 461.25794 |
| | methylpiperazin-1- | m + H = 462.2, >98% @215 | |
| | yl)phenyl]imidazo[1,2-a]pyridin-3- | and 254 nm | |
| | yl]quinoline; 2,2,2-trifluoroacetic acid | | |
| 117 | 2-butyl-4-[7-[4-(4-methylpiperazin-1- | LCMS: RT = 0.656 min, | 475.27359 |
| | yl)phenyl]imidazo[1,2-a]pyridin-3- | m + H = 476.2, >98% @215 | |
| | yl]quinoline; 2,2,2-trifluoroacetic acid | and 254 nm | |
| 118 | 6-ethoxy-2-methyl-4-[7-[4-(4- | LCMS: RT = 0.585 min, | 477.25287 |
| | methylpiperazin-1- | m + H = 478.2, >98% @215 | |
| | yl)phenyl]imidazo[1,2-a]pyridin-3- | and 254 nm | |
| | yl]quinoline; 2,2,2-trifluoroacetic acid | | |
| 119 | 7-[4-(4-methylpiperazin-1-yl)phenyl]-3- | LCMS: RT = 0.684 min, | 388.17218 |
| | (5-methyl-2-thienyl)imidazo[1,2- | m + H = 389.2, >98% @215 | |
| | a]pyridine; 2,2,2-trifluoroacetic acid | and 254 nm | |
| 120 | 3-(5-chloro-2-thienyl)-7-[4-(4- | LCMS: RT = 0.716 min, | 408.11755 |
| | methylpiperazin-1- | m + H = 109.2, >98% @215 | |
| | yl)phenyl]imidazo[1,2-a]pyridine; 2,2,2- | and 254 nm | |
| | trifluoroacetic acid | | |
| 121 | 8-[7-[4-(4-methylpiperazin-1- | LCMS: RT = 0.580 min, | 435.2059 |
| | yl)phenyl]imidazo[1,2-a]pyridin-3- | m + H = 436.2, >98% @215 | |
| | yl]quinolin-2-ol; 2,2,2-trifluoroacetic | and 254 nm | |
| | acid | | |
| 122 | 4-[7-[6-(4-cyclopropylpiperazin-1-yl)-3- | LCMS: RT = 0.513 min, | 446.22189 |
| | pyridyl]imidazo[1,2-a]pyridin-3- | m + H = 447.2, >98% @215 | |
| | yl]quinoline; 2,2,2-trifluoroacetic acid | and 254 nm | |
| 123 | 4-[7-[6-(6,6-difluoro-2- | LCMS: RT = 0.600 min, | 453.17651 |
| | azaspiro[3.3]heptan-2-yl)-3- | m + H = 454.2, >95% @215 | |
| | pyridyl]imidazo[1,2-a]pyridin-3- | and 254 nm | |
| | yl]quinoline; 2,2,2-trifluoroacetic acid | | |
| 124 | 4-[7-[6-[(3R)-3-methylpiperazin-1-yl]- | LCMS: RT = 0.485 min, | 420.20624 |
| | 3-pyridyl]imidazo[1,2-a]pyridin-3- | m + H = 421.2, >98% @215 | |
| | yl]quinoline; 2,2,2-trifluoroacetic acid | and 254 nm | |
| 125 | 4-[7-[6-[(3S)-3-methylpiperazin-1-yl]-3- | LCMS: RT = 0.497 min, | 420.20624 |
| | pyridyl]imidazo[1,2-a]pyridin-3- | m + H = 421.2, >98% @215 | |
| | yl]quinoline; 2,2,2-trifluoroacetic acid | and 254 nm | |
| 126 | (2S)-2-methyl-4-[5-[3-(4- | LCMS: RT = 0.573 min, | 421.19025 |
| | quinolyl)imidazo[1,2-a]pyridin-7-yl]-2- | m + H = 422.2, >98% @215 | |
| | pyridyl]morpholine; 2,2,2- | and 254 nm | |
| | trifluoroacetic acid | | |
| 127 | 4-[7-[6-(1,7-diazaspiro[4.4]nonan-7-yl)- | LCMS: RT = 0.514 min, | 446.22189 |
| | 3-pyridyl]imidazo[1,2-a]pyridin-3- | m + H = 447.2, >98% @215 | |
| | yl]quinoline; 2,2,2-trifluoroacetic acid | and 254 nm | |
| 128 | 4-[7-[6-(2-piperazin-1-ylethoxy)-3- | LCMS: RT = 0.460 min, | 450.2168 |
| | pyridyl]imidazo[1,2-a]pyridin-3- | m + H = 451.2, >98% @215 | |
| | yl]quinoline; 2,2,2-trifluoroacetic acid | and 254 nm | |
| 129 | (2R,6S)-2,6-dimethyl-4-[5-[3-(4- | LCMS: RT = 0.624 min, | 435.2059 |
| | quinolyl)imidazo[1,2-a]pyridin-7-yl]-2- | m + H = 436.2, >98% @215 | |
| | pyridyl]morpholine; 2,2,2- | and 254 nm | |
| | trifluoroacetic acid | | |
| 130 | 2,2,2-trifluoroacetic acid; 4-[7-[6- | LCMS: RT = 0.557 min, | 448.23755 |
| | [(3S,5R)-3,4,5-trimethylpiperazin-1-yl]- | m + H = 449.2, 90% @215 | |
| | 3-pyridyl]imidazo[1,2-a]pyridin-3- | and 254 nm | |
| | yl]quinoline | | |
| 131 | 4-[7-[6-(2,3,3a,4,6,6a-hexahydro-1H- | LCMS: RT = 0.382 min, | 432.20624 |
| | pyrrolo[3,4-c]pyrrol-5-yl)-3- | m + H = 433.2, >90% @215 | |
| | pyridyl]imidazo[1,2-a]pyridin-3- | and 254 nm | |
| | yl]quinoline; 2,2,2-trifluoroacetic acid | 1H NMR d-DMSO: | |
| | | 400 MHz, >90% | |
| 132 | 8-[7-[4-(4-methylpiperazin-1- | LCMS: Rt = 0.537 min, | 421.20148 |
| | yl)phenyl]imidazo[1,2-a]pyridin-3- | M + H 422.2: 98% @ 215 | |
| | yl]pyrido[3,4-b]pyrazine; 2,2,2- | and 254 nm | |
| | trifluoroacetic acid | | |
| 133 | 4-[7-[4-(4-methylpiperazin-1- | LCMS: Rt = 0.580 min, | 435.2059 |
| | yl)phenyl]imidazo[1,2-a]pyridin-3- | M + H = 436.2; >98% @ 215 | |
| | yl]quinolin-2-ol; 2,2,2-trifluoroacetic | and 254 nm | |
| | acid | | |
| 134 | 7-[7-[4-(4-methylpiperazin-1- | LCMS: Rt = 0.608 min, | 425.16742 |
| | yl)phenyl]imidazo[1,2-a]pyridin-3-yl]- | M + H = 426.2; >98% @ 215 | |
| | 1,3-benzothiazole; 2,2,2-trifluoroacetic | and 254 nm | |
| | acid | | |
| 135 | 6-methyl-8-[7-[4-(4-methylpiperazin-1- | LCMS: Rt = 0.556 min, | 423.21713 |
| | yl)phenyl]imidazo[1,2-a]pyridin-3-yl]- | M + H = 424.2; >98% @ 215 | |
| | [1,2,4]triazolo[1,5-a]pyridine; 2,2,2- | an 254 nm | |
| | trifluoroacetic acid | | |
| 136 | 8-[7-[4-(1-methyl-4- | LCMS: Rt = 0.601 min, | 420.20624 |
| | piperidyl)phenyl]imidazo[1,2-a]pyridin- | M + H = 421.2; >98% @ 254 | |
| | 3-yl]pyrido[3,4-b]pyrazine; 2,2,2- | nm | |
| | trifluoroacetic acid | | |
| 137 | 7-[7-[4-(1-methyl-4- | LCMS: Rt = 0.562 min, | 425.16742 |
| | piperidyl)phenyl]imidazo[1,2-a]pyridin- | M + H = 426.2; >98% @ 215 | |
| | 3-yl]thieno[3,2-d]pyrimidine; 2,2,2- | and 254 nm | |
| | trifluoroacetic acid | | |
| 138 | 2-methyl-4-[7-[4-(4-methylpiperazin-1- | LCMS: Rt = 0.700 min, | 501.21402 |
| | yl)phenyl]imidazo[1,2-a]pyridin-3-yl]- | M + H = 502.2; >98% @ 215 | |
| | 6-(trifluoromethyl)quinoline; 2,2,2- | and 254 nm | |
| | trifluoroacetic acid | | |
| 139 | 6-methoxy-2-methyl-4-[7-[4-(4- | LCMS: Rt = 0.755 min, | 527.24335 |
| | pyrimidin-2-ylpiperazin-1- | M + H = 528.2; >98% @ 215 | |
| | yl)phenyl]imidazo[1,2-a]pyridin-3- | and 254 nm | |
| | yl]quinoline; 2,2,2-trifluoroacetic acid | | |
| 140 | 4-[7-[4-[4-(4,6-dimethoxypyrimidin-2- | LCMS: Rt = 0.900 min, | 587.26447 |
| | yl)piperazin-1-yl]phenyl]imidazo[1,2- | M + H = 588.2; >98% @215 | |
| | a]pyridin-3-yl]-6-methoxy-2-methyl- | and 254 nm | |
| | quinoline; 2,2,2-trifluoroacetic acid | | |
| 141 | 6-methoxy-2-methyl-4-[7-[4-[4-(5- | LCMS: Rt = 0.843 min, | 569.29028 |
| | propylpyrimidin-2-yl)piperazin-1- | M + H = 570.2; >98% @15 | |
| | yl]phenyl]imidazo[1,2-a]pyridin-3- | and 254 nm | |
| | yl]quinoline; 2,2,2-trifluoroacetic acid | | |
| 142 | 4-[7-[4-[4-(5-fluoropyrimidin-2- | LCMS Rt = 0.858 MIN, | 545.23395 |
| | yl)piperazin-1-yl]phenyl]imidazo[1,2- | M + H = 546.2; >98% @ 215 | |
| | a]pyridin-3-yl]-6-methoxy-2-methyl- | AND 254 nm | |
| | quinoline; 2,2,2-trifluoroacetic acid | | |
| 143 | 4-[7-[6-(4-cyclopropylpiperazin-1-yl)-3- | LCMS: Rt = 0.484 min, | 460.23755 |
| | pyridyl]imidazo[1,2-a]pyridin-3-yl]-2- | M + H = 461.2; >98% @ 215 | |
| | methyl-quinoline; 2,2,2-trifluoroacetic | and 254 nm | |
| | acid | | |
| 144 | 4-[7-[6-(4-isopropylpiperazin-1-yl)-3- | LCMS: Rt = 0.490 min, | 462.2532 |
| | pyridyl]imidazo[1,2-a]-alpyridin-3-yl]-2- | M + H = 463.2; >98% @ 215 | |
| | methyl-quinoline; 2,2,2-trifluoroacetic | and 254 nm | |
| | acid | | |
| 145 | 4-[7-[6-(4-ethyl-3,3-dimethyl-piperazin- | LCMS: Rt = 0.514 min, | 476.26883 |
| | 1-yl)-3-pyridyl]imidazo[1,2-a]pyridin-3- | M + H = 477.3; >98% @ 215 | |
| | yl]-2-methyl-quinoline; 2,2,2- | and 254 nm | |
| | trifluoroacetic acid | | |
| 146 | 4-[7-[6-(6,6-difluoro-2- | LCMS: Rt = 0.577 min, | 467.19214 |
| | azaspiro[3.3]heptan-2-yl)-3- | M + H = 468.2; >98% @ 215 | |
| | pyridyl]imidazo[1,2-a]pyridin-3-yl]-2- | and 254 nm | |
| | methyl-quinoline; 2,2,2-trifluoroacetic | | |
| | acid | | |
| 147 | (2S,6R)-2,6-dimethyl-4-[5-[3-(2- | LCMS: Rt = 0.586 min, | 449.22156 |
| | methyl-4-quinolyl)imidazo[1,2- | M + H = 450.2; >98% @ 215 | |
| | a]pyridin-7-yl]-2-pyridyl]morpholine; | and 254 nm | |
| | 2,2,2-trifluoroacetic acid | | |
| 148 | 2-methyl-4-[7-[5-methyl-6-[(3S)-3- | LCMS: Rt = 0.558 min, | 448.23755 |
| | methylpiperazin-1-yl]-3- | M + H = 449.2; >94% @ 215 | |
| | pyridyl]imidazo[1,2-a]pyridin-3- | and >96% @ 254 nm | |
| | yl]quinoline; 2,2,2-trifluoroacetic acid | | |
| 149 | 4-[7-(6-methoxy-5-methyl-3- | LCMS: Rt = 0.442 min, | 380.1637 |
| | pyridyl)imidazo[1,2-a]pyridin-3-yl]-2- | M + H = 369.0; >98% @ 215 | |
| | methyl-quinoline; 2,2,2-trifluoroacetic | and 254 nm | |
| | acid | | |
| 150 | 2-methyl-4-[7-[5-methyl-6-[(3R)-3- | LCMS: Rt = 0.551 min, | 448.23755 |
| | methylpiperazin-1-yl]-3- | M + H = 449.2; >98% @ 215 | |
| | pyridyl]imidazo[1,2-a]pyridin-3- | and 254 nm | |
| | yl]quinoline; 2,2,2-trifluoroacetic acid | | |
| 151 | 4-[7-[6-(4-isopropylpiperazin-1-yl)-5- | LCMS: Rt = 0.580 min, | 476.26883 |
| | methyl-3-pyridyl]imidazo[1,2-a]pyridin- | M + H = 477.2; >98% @ 215 | |
| | 3-yl]-2-methyl-quinoline; 2,2,2- | and 254 nm | |
| | trifluoroacetic acid | | |
| 152 | 4-[7-[6-(4-cyclopropylpiperazin-1-yl)-5- | LCMS: Rt = 0.568 min, | 474.2532 |
| | methyl-3-pyridyl]imidazo[1,2-a]pyridin- | M + H = 475.2; >98% @215 | |
| | 3-yl]-2-methyl-quinoline; 2,2,2- | and 254 nm | |
| | trifluoroacetic acid | | |
| 153 | 4-[7-[6-(6,6-difluoro-2- | LCMS: Rt = 0.610 min, | 481.20779 |
| | azaspiro[3.3]heptan-2-yl)-5-methyl-3- | M + H = 482.2; >98% @15 | |
| | pyridyl]imidazo[1,2-a]pyridin-3-yl]-2- | and 254 nm | |
| | methyl-quinoline; 2,2,2-trifluoroacetic | | |
| | acid | | |
| 154 | 4-[7-[6-(2,6-diazaspiro[3.4]octan-6-yl)- | LCMS(basic method): | 446.22189 |
| | 3-pyridyl]imidazo[1,2-a]pyridin-3-yl]-2- | Rt = 1.121 min, | |
| | methyl-quinoline; 2,2,2-trifluoroacetic | M + H = 447.2; >98% @ 215 | |
| | acid | and 254 nm | |
| 155 | 4-[7-[6-(2,3,3a,4,6,6a-hexahydro-1H- | LCMS: Rt = 0.464 min, | 446.22189 |
| | pyrrolo[3,4-c]pyrrol-5-yl)-3- | M + H = 447.2; >98% @ 215 | |
| | pyridyl]imidazo[1,2-a]pyridin-3-yl]-2- | and 254 nm | |
| | methyl-quinoline; 2,2,2-trifluoroacetic | | |
| | acid | | |
| 156 | 4-[7-[6-(1,7-diazaspiro[4.4]nonan-7-yl)- | LCMS: Rt = 0.422 min, | 460.23755 |
| | 3-pyridyl]imidazo[1,2-a]pyridin-3-yl]-2- | M + H = 461.2; >98% @ 215 | |
| | methyl-quinoline; 2,2,2-trifluoroacetic | and 254 nm | |
| | acid | | |
| 157 | 4-[7-[6-(2,6-diazaspiro[3.3]heptan-2- | LCMS: Rt = 0.461 min, | 432.20624 |
| | yl)-3-pyridyl]imidazo[1,2-a]pyridin-3- | M + H = 433.2; >98% @ 215 | |
| | yl]-2-methyl-quinoline; 2,2,2- | and 254 nm | |
| | trifluoroacetic acid | | |
| 158 | 2-methyl-4-[7-(5-methyl-6-piperazin-1- | LCMS: Rt = 0.567 min, | 434.22189 |
| | yl-3-pyridyl)imidazo[1,2-a]pyridin-3- | M + H = 434.2; >98% @215 | |
| | yl]quinoline; 2,2,2-trifluoroacetic acid | and 254 nm | |
| 159 | 4-[7-[6-(1,7-diazaspiro[4.4]nonan-7-yl)- | LCMS: Rt = 0.455 min, | 474.2532 |
| | 5-methyl-3-pyridyl]imidazo[1,2- | M + H = 475.2; >98% @ 215 | |
| | a]pyridin-3-yl]-2-methyl-quinoline; | and 254 nm | |
| | 2,2,2-trifluoroacetic acid | | |
| 160 | 4-[7-[6-(2,3,3a,4,6,6a-hexahydro-1H- | LCMS: Rt = 0.501 min, | 460.23755 |
| | pyrrolo[3,4-c]pyrrol-5-yl)-5-methyl-3- | M + H = 461.2; >98% @ 215 | |
| | pyridyl]imidazo[1,2-a]pyridin-3-yl]-2- | and 254 nm | |
| | methyl-quinoline; 2,2,2-trifluoroacetic | | |
| | acid | | |
| 161 | 2-methyl-4-[7-[4-(1-methyl-4- | LCMS: Rt = 0.746 min, | 500.21878 |
| | piperidyl)phenyl]imidazo[1,2-a]pyridin- | M + H = 501.2; >91% @ 215 | |
| | 3-yl]-6-(trifluoromethyl)quinoline; | nm and >96% @ 254 nm | |
| | 2,2,2-trifluoroacetic acid | | |
| 162 | 6-fluoro-2-methyl-4-[7-[6-[(3R)-3- | LCMS: Rt = 0.558 min, | 452.21246 |
| | methylpiperazin-1-yl]-3- | M + H = 453.2; >98% @ 215 | |
| | pyridyl]imidazo[1,2-a]pyridin-3- | and 254 nm | |
| | yl]quinoline; 2,2,2-trifluoroacetic acid | | |
| 163 | 4-[7-[6-(1,7-diazaspiro[4.4]nonan-7-yl)- | LCMS: Rt = 0.542 min, | 478.22812 |
| | 3-pyridyl]imidazo[1,2-a]pyridin-3-yl]-6- | M + H = 479.2; >98% @ 215 | |
| | fluoro-2-methyl-quinoline; 2,2,2- | and 254 nm | |
| | trifluoroacetic acid | | |
| 164 | 4-[7-[6-(4-ethyl-3,3-dimethyl-piperazin- | LCMS: Rt = 0.603 min, | 494.25943 |
| | 1-yl)-3-pyridyl]imidazo[1,2-a]pyridin-3- | M + H = 495.2; >98% @ 215 | |
| | yl]-6-fluoro-2-methyl-quinoline; 2,2,2- | and 254 nm | |
| | trifluoroacetic acid | | |
| 165 | 4-[7-[6-(4-cyclopropylpiperazin-1-yl)-3- | LCMS: Rt = 0.576 min, | 478.22812 |
| | pyridyl]imidazo[1,2-a]pyridin-3-yl]-6- | M + H = 479.2; >95% @ 215 | |
| | fluoro-2-methyl-quinoline; 2,2,2- | and >94% @ 254 nm | |
| | trifluoroacetic acid | | |
| 166 | 4-[7-[6-(2,3,3a,4,6,6a-hexahydro-1H- | LCMS: Rt = 0.500 min, | 464.21246 |
| | pyrrolo[3,4-c]pyrrol-5-yl)-3- | M + H = 465.2; >98% @ 215 | |
| | pyridyl]imidazo[1,2-a]pyridin-3-yl]-6- | and 254 nm | |
| | fluoro-2-methyl-quinoline; 2,2,2- | | |
| | trifluoroacetic acid | | |
| 167 | 6-fluoro-4-[7-[4-[1-(5-fluoropyrimidin- | LCMS: RT = 1.028 min, | 532.21869 |
| | 2-yl)-4-piperidyl]phenyl]imidazo[1,2- | m + H = 533.2, >98% @215 | |
| | a]pyridin-3-yl]-2-methyl-quinoline; | and 254 nm | |
| | 2,2,2-trifluoroacetic acid | | |
| 168 | 6-fluoro-4-[7-[4-(1-isopropyl-4- | LCMS: RT = 0.675 min, | 478.25327 |
| | piperidyl)phenyl]imidazo[1,2-a]pyridin- | m + H = 479.2, >98% @215 | |
| | 3-yl]-2-methyl-quinoline; 2,2,2- | and 254 nm | |
| | trifluoroacetic acid | | |
| 169 | 6-fluoro-4-[7-[4-(1-isobutyl-4- | LCMS: RT = 0.702 min, | 492.26892 |
| | piperidyl)phenyl]imidazo[1,2-a]pyridin- | m + H = 493.2, >98% | |
| | 3-yl]-2-methyl-quinoline; 2,2,2- | @215and 254 nm | |
| | trifluoroacetic acid | | |
| 170 | 7-[7-[4-(1-isobutyl-4- | LCMS: RT = 0.672 min, | 467.21436 |
| | piperidyl)phenyl]imidazo[1,2-a]pyridin- | m + H = 468.2, >98% @215 | |
| | 3-yl]thieno[3,2-d]pyrimidine; 2,2,2- | and 254 nm | |
| | trifluoroacetic acid | | |
| 171 | 6-fluoro-4-[7-[4-[1-(2-methoxyethyl)-4- | LCMS: RT = 0.651 min, | 494.2482 |
| | piperidyl]phenyl]imidazo[1,2-a]pyridin- | m + H = 495.2, >94% @215 | |
| | 3-yl]-2-methyl-quinoline; 2,2,2- | and 254 nm | |
| | trifluoroacetic acid | | |
| 172 | 7-[7-[4-[1-(2-methoxyethyl)-4- | LCMS: RT = 0.616 min, | 469.19363 |
| | piperidyl]phenyl]imidazo[1,2-a]pyridin- | m + H = 470.2, >98% @215 | |
| | 3-yl]thieno[3,2-d]pyrimidine; 2,2,2- | and 254 nm | |
| | trifluoroacetic acid | | |
| 173 | 6-fluoro-4-[7-[5-(4-isopropylpiperazin- | LCMS: RT = 0.608 min, | 481.23901 |
| | 1-yl)pyrazin-2-yl]imidazo[1,2- | m + H = 482.2, >98% @215 | |
| | a]pyridin-3-yl]-2-methyl-quinoline | and 254 nm | |
| 174 | 6,8-difluoro-4-[7-[5-(4- | LCMS: RT = 0.664 min, | 499.22958 |
| | isopropylpiperazin-1-yl)pyrazin-2- | m + H = 500.2, >98% @215 | |
| | yl]imidazo[1,2-a]pyridin-3-yl]-2- | and 254 nm | |
| | methyl-quinoline | | |
| 175 | 6-fluoro-2-methyl-4-[7-[6-(4- | M + H = 453.3; >98% by | 452.21246 |
| | methylpiperazin-1-yl)-3- | LCMS @215 and @254 | |
| | pyridyl]imidazo[1,2-a]pyridin-3- | HPLC RT = 0.614 min. | |
| | yl]quinoline | | |
| 176 | 4-[7-[6-[(3R)-3,4-dimethylpiperazin-1- | M + H = 467.3; >98% by | 466.22812 |
| | yl]-3-pyridyl]imidazo[1,2-a]pyridin-3- | LCMS @215 and @254 | |
| | yl]-6-fluoro-2-methyl-quinoline | HPLC RT = 0.610 min. | |
| 177 | 6-fluoro-2-methyl-4-[7-[6-[(3R,5S)- | M + H = 481.3; >98% by | 480.24377 |
| | 3,4,5-trimethylpiperazin-1-yl]-3- | LCMS @215 and @254 | |
| | pyridyl]imidazo[1,2-a]pyridin-3- | HPLC RT = 0.629 min. | |
| | yl]quinoline | | |
| 178 | 2-[5-[3-(6-fluoro-2-methyl-4- | M + H = 494.3; >98% by | 493.22778 |
| | quinolyl)imidazo[1,2-a]pyridin-7-yl]-2- | LCMS @215 and @254 | |
| | pyridyl]-8-oxa-2-azaspiro[4.5]decane | HPLC RT = 0.622 min. | |
| 179 | 1-[5-[3-(6-fluoro-2-methyl-4- | M + H = 435.3; >98% by | 434.16553 |
| | quinolyl)imidazo[1,2-a]pyridin-7-yl]-2- | LCMS @215 and @254 | |
| | pyridyl]azetidine-3-carbonitrile | HPLC RT = 0.572 min. | |
| 180 | 6-fluoro-2-methyl-4-[7-[6-(4- | M + H = 516.3; >98% by | 515.17914 |
| | methylsulfonyl-1-piperidyl)-3- | LCMS @215 and @254 | |
| | pyridyl]imidazo[1,2-a]pyridin-3- | HPLC RT = 0.587 min. | |
| | yl]quinoline | | |
| 181 | 3-[5-[3-(6-fluoro-2-methyl-4- | M + H = 466.3; >98% by | 465.19647 |
| | quinolyl)imidazo[1,2-a]pyridin-7-yl]-2- | LCMS @215 and @254 | |
| | pyridyl]-8-oxa-3- | HPLC RT = 0.641 min. | |
| | azabicyclo[3.2.1]octane | | |
| 182 | 1-[4-[5-[3-(6-fluoro-2-methyl-4- | M + H = 481.3; >98% by | 480.2074 |
| | quinolyl)imidazo[1,2-a]pyridin-7-yl]-2- | LCMS @215 and @254 | |
| | pyridyl]piperazin-1-yl]ethanone | HPLC RT = 0.578 min. | |
| 183 | 4-[7-[6-(2,6-diazaspiro[3.3]heptan-2- | M + H = 451.2; >98% by | 450.19681 |
| | yl)-3-pyridyl]imidazo[1,2-a]pyridin-3- | LCMS @215 and @254 | |
| | yl]-6-fluoro-2-methyl-quinoline | HPLC RT = 0.633 min. | |
| 184 | N-[(1R,2R)-2-hydroxycyclohexyl]-4-[7- | M + H = 516.4; >98% by | 515.23547 |
| | [4-(4-methylpiperazin-1- | LCMS @254 HPLC RT = | |
| | yl)phenyl]imidazo[1,2-a]pyridin-3- | 0.700 min. | |
| | yl]thiophene-2-carboxamide | | |
| 185 | N-[(1R,2R)-2-hydroxycyclopentyl]-4- | M + H = 502.3; >98% by | 501.21985 |
| | [7-[4-(4-methylpiperazin-1- | LCMS @215 and @254 | |
| | yl)phenyl]imidazo[1,2-a]pyridin-3- | HPLC RT = 0.652 min. | |
| | yl]thiophene-2-carboxamide | | |
| 186 | N-(1-cyclopropyl-2-hydroxy-ethyl)-4- | M + H = 502.3; >98% by | 501.21985 |
| | [7-[4-(4-methylpiperazin-1- | LCMS @215 and @254 | |
| | yl)phenyl]imidazo[1,2-a]pyridin-3- | HPLC RT = 0.564 min. | |
| | yl]thiophene-2-carboxamide | | |
| 187 | 6-fluoro-2-methyl-4-[7-[4-[(3S,5R)- | M + H = 480.4; >98% by | 479.24854 |
| | 3,4,5-trimethylpiperazin-1- | LCMS @215 and @254 | |
| | yl]phenyl]imidazo[1,2-a]pyridin-3- | HPLC RT = 0.638 min. | |
| | yl]quinoline | | |
| 188 | 4-[7-[6-(7-azabicyclo[2.2.1]heptan-7- | M + H = 450.3; >95% by | 449.20157 |
| | yl)-3-pyridyl]imidazo[1,2-a]pyridin-3- | LCMS @215 and @254 | |
| | yl]-6-fluoro-2-methyl-quinoline | HPLC RT = 0.650 min. | |
| 189 | (2R,6S)-4-[5-[3-(6-fluoro-2-methyl-4- | M + H = 468.3; >98% by | 467.21213 |
| | quinolyl)imidazo[1,2-a]pyridin-7-yl]-2- | LCMS @215 and @254 | |
| | pyridyl]-2,6-dimethyl-morpholine | HPLC RT = 0.649 min. | |
| 190 | 6-fluoro-2-methyl-4-[7-[6-(4-pyrimidin- | M + H = 517.3; >90% by | 516.21863 |
| | 2-ylpiperazin-1-yl)-3- | LCMS @215 and @254 | |
| | pyridyl]imidazo[1,2-a]pyridin-3- | HPLC RT = 0.655 min. | |
| | yl]quinoline | | |
| 191 | 6-fluoro-4-[7-[6-(4-isobutylpiperazin-1- | M + H = 495.4; >95% by | 494.25943 |
| | yl)-3-pyridyl]imidazo[1,2-a]pyridin-3- | LCMS @215 and @254 | |
| | yl]-2-methyl-quinoline | HPLC RT = 0.597 min. | |
| 192 | 6-fluoro-4-[7-[6-[4-(2-methoxyethyl)-1- | M + H = 496.4; >99% by | 495.24344 |
| | piperidyl]-3-pyridyl]imidazo[1,2- | LCMS @215 and @254 | |
| | a]pyridin-3-yl]-2-methyl-quinoline | HPLC RT = 0.656 min. | |
| 193 | 6,8-difluoro-2-methyl-4-[7-[4-[(3S,5R)- | M + H = 498.4; >95% by | 497.23911 |
| | 3,4,5-trimethylpiperazin-1- | LCMS @215 and @254 | |
| | yl]phenyl]imidazo[1,2-a]pyridin-3- | HPLC RT = 0.659 min. | |
| | yl]quinoline | | |
| 194 | 7-[7-[4-[(3S,5R)-3,4,5- | M + H = 455.3; >98% by | 454.19397 |
| | trimethylpiperazin-1- | LCMS @215 and @254 | |
| | yl]phenyl]imidazo[1,2-a]pyridin-3- | HPLC RT = 0.602 min. | |
| | yl]thieno[3,2-d]pyrimidine | | |
| 195 | 2-methyl-4-[7-[4-[(3S,5R)-3,4,5- | M + H = 462.4; >90% by | 461.25794 |
| | trimethylpiperazin-1- | LCMS @215 and @254 | |
| | yl]phenyl]imidazo[1,2-a]pyridin-3- | HPLC RT = 0.500 min. | |
| | yl]quinoline | | |
| 196 | 1-[(2S)-4-[4-[3-(6-fluoro-2-methyl-4- | M + H = 494.3; >98% by | 493.22778 |
| | quinolyl)imidazo[1,2-a]pyridin-7- | LCMS @215 and @254 | |
| | yl]phenyl]-2-methyl-piperazin-1- | HPLC RT = 0.760 min. | |
| | yl]ethanone | | |
| 197 | 1-[(2S)-2-methyl-4-[4-[3-(2-methyl-4- | M + H = 476.4; >98% by | 475.23721 |
| | quinolyl)imidazo[1,2-a]pyridin-7- | LCMS @215 and @254 | |
| | yl]phenyl]piperazin-1-yl]ethanone | HPLC RT = 0.624 min. | |
| 198 | 1-[(2S)-4-[4-[3-(6,8-difluoro-2-methyl- | M + H = 512.4; >90% by | 511.21835 |
| | 4-quinolyl)imidazo[1,2-a]pyridin-7- | LCMS @215 and @254 | |
| | yl]phenyl]-2-methyl-piperazin-1- | HPLC RT = 0.794 min. | |
| | yl]ethanone | | |
| 199 | 1-[(2S)-2-methy1-4-[4-(3-thieno[3,2- | M + H = 469.3; >90% by | 468.17322 |
| | d]pyrimidin-7-ylimidazo[1,2-a]pyridin- | LCMS @215 and @254 | |
| | 7-yl)phenyl]piperazin-1-yl]ethanone | HPLC RT = 0.682 min. | |
| 200 | 5-[7-(4-piperazin-1- | M + H = 422.3; >98% by | 421.19025 |
| | ylphenyl)imidazo[1,2-a]pyridin-3-yl]- | LCMS @215 and @254 | |
| | 1H-quinolin-2-one | HPLC RT = 0.504 min. | |
| 201 | (2S,6R)-4-[4-[3-(6,8-difluoro-2-methyl- | M + H = 485.3; >98% by | 484.20746 |
| | 4-quinolyl)imidazo[1,2-a]pyridin-7- | LCMS @215 and @254 | |
| | yl]phenyl]-2,6-dimethyl-morpholine | HPLC RT = 0.894 min. | |
| 202 | (2S,6R)-4-[4-[3-(6-fluoro-2-methyl-4- | M + H = 469.5; >98% by | 466.21689 |
| | quinolyl)imidazo[1,2-a]pyridin-7- | LCMS @215 and @254 | |
| | yl]phenyl]-2,6-dimethyl-morpholine | HPLC RT = 0.977 min. | |
| 203 | (2S,6R)-2,6-dimethyl-4-[4-[3-(2- | M + H = 449.2; >96% by | 448.22632 |
| | methyl-4-quinolyl)imidazo[1,2- | LCMS @215 and @254 | |
| | a]pyridin-7-yl]phenyl]morpholine | HPLC RT = 0.998 min. | |
| 204 | 6,8-difluoro-2-methyl-4-[7-[4-[(3R)-3- | M + H = 470.3; >98% by | 469.20779 |
| | methylpiperazin-1- | LCMS @215 and @254 | |
| | yl]phenyl]imidazo[1,2-a]pyridin-3- | HPLC RT = 0.751 min. | |
| | yl]quinoline | | |
| 205 | 7-[7-[4-[(3R)-3-methylpiperazin-1- | M + H = 427.3; >98% by | 426.16266 |
| | yl]phenyl]imidazo[1,2-a]pyridin-3- | LCMS @215 and @254 | |
| | yl]thieno[3,2-d]pyrimidine | HPLC RT = 0.623 min. | |

### Example 2: Biological Assays

Tables 3-5 summarize the results of assays used to identify and evaluate embodiments of the present invention.

TABLE 3
Biological Assay data for selected compounds

| | Compound | Compound | Compound | Compound | Compound | Compound |
|---|---|---|---|---|---|---|
| | 17 | 20 | 25 | 48 | 95 | 168 |
| | nM | | | | | |
| BMP4 | 28.5 | 74 | 16.5 | 185 | 26.5 | 392 |
| caALK2 | 1.8 | 30.2 | 1 | 572.5 | 87 | 127 |
| caALK1 | >1,000 | 2,165 | 113.2 | 5,565 | 594 | 627 |
| Selectivity | Ki (nM) | | | | | |
| ALK1 | 308.8 | 166 | 39.1 | 4,275 | 831 | 179 |
| ALK2 | 32.2 | 10.9 | 8.0 | 338 | 9.3 | 7.2 |
| ALK2 | 8.8 | 5.6 | 3.8 | nd | | |
| (R206H) | | | | | | |
| ALK3 | <5.1 | <5.1 | <5.1 | <5.1 | <5.1 | 34.6 |
| ALK4 | 596.1 | 559 | 464 | 144 | 292 | nd |
| ALK5 | 1,285 | 1,267 | 1,260 | 319 | 822 | nd |
| ALK6 | 40.9 | 29.4 | 18.4 | 965 | 97.8 | nd |
| BMPR2 | >50,000 | >50,000 | >10,000 | >30,000 | nd | nd |
| TGFβR2 | 170.7 | 587.2 | 1250 | 196 | 134 | nd |
| AMPK | 18,600 | 13,590 | >10,000 | 8,703 | nd | nd |
| KDR | 20,560 | 13,410 | 5,930 | 8,730 | 3,260 | nd |
| PDGFβ | >10,000 | 7,450 | 3,750 | nd | nd | nd |

nd = not determined

TABLE 4
In vitro DMPK data for selected compounds.

| Compound | Compound | Compound | Compound | Compound | Compound |
|---|---|---|---|---|---|
| 17 | 20 | 25 | 48 | 95 | 168 |

Intrinsic clearance (mL/min/kg)

| | | | | | | |
|---|---|---|---|---|---|---|
| hCL_{INT} | 0.82 | 12.3 | 28.9 | 294 | 36.6 | 8.6 |
| hCL_{HEP} | 0.79 | 7.7 | 12.2 | 19.6 | 13.3 | 6.1 |
| mCL_{INT} | 131 | 207 | 87.7 | 349 | 107 | 0.0 |
| mCL_{HEP} | 53.4 | 62.7 | 44.4 | 71.5 | 48.9 | 0.0 |
| rCL_{INT} | 347 | 370 | 174 | nd | nd | 18.2 |
| rCL_{HEP} | 58.2 | 58.9 | 49.9 | nd | nd | 14.5 |

Plasma protein binding (% Fᵤ)

| | | | | | | |
|---|---|---|---|---|---|---|
| Human | 1.4 | 1.0 | 1.4 | 1.2 | 1.5 | 2.1 |
| Mouse | 2.2 | 1.0 | 1.8 | 5.8 | 0.6 | 0.9 |
| Rat | 3.0 | 0.9 | 0.8 | nd | nd | 1.4 |
| Dog | 6.2 | 4.5 | 4.3 | nd | nd | nd |
| Cyno | 2.7 | 4.0 | 3.1 | nd | nd | nd |

CYP Inhibition (μM)

| | | | | | | |
|---|---|---|---|---|---|---|
| CYP2C9 | >20 | >20 | >20 | nd | nd | nd |
| CYP2D6 | >20 | >20 | >20 | nd | nd | nd |
| CYP3A4 | 5.7 | 12.7 | 16.8 | nd | nd | nd |

nd = not determined

TABLE 5
In vivo PK data for selected compounds.

| | | |
|---|---|---|
| Compound 17 | Compound 20 | Compound 25 |

In vivo PK (rat)
IV PK (0.5 mg/kg)

| | | | |
|---|---|---|---|
| CL (mL/min/kg) | 16.7 | 12.9 | 17.1 |
| T_{1/2} (h) | 5.4 | 5.9 | 5.9 |
| Cₘₐₓ (ng/mL) | 83.6 | 144 | 133.2 |
| C₀ (ng/mL) | 93.8 | 151 | 143.0 |
| Vd (L/kg) | 7.7 | 5.8 | 8.8 |
| Vdss (L/kg) | 6.8 | 5.5 | 7.3 |
| AUC₀₋ₜ (hr*ng/mL) | 456.3 | 435 | 464.1 |
| AUC_{0-∞} (hr*ng/mL) | 503.9 | 701 | 489.6 |

PO PK (3 mg/kg)

| T_{1/2} (h) | 3.8 | | 3.9 | 4.7 |
|---|---|---|---|---|
| Cₘₐₓ (ng/mL) | 95.7 | | 163 | 127.7 |
| Tmax (hr) | 4 | | 4 | 4 |
| AUC₀₋ₜ (hr*ng/mL) | 905.4 | | 1,637 | 1,235.9 |
| AUC_{0-∞} (hr*ng/mL) | 920.5 | | 1,683 | 1,289 |
| % F | 29.8 | | 40 | 45.3 |
| Solubility (μg/mL) | 1 h | 24 h | | |
| FaSSIF | 20 | 17 | | |
| SGF | 914 | 893 | | |
| Kinetic | 17.8 | | | |

### Example 3—Mutagenic Potential of Compounds of Formula 1

Compound 17 was evaluated in the Ames test to assess the mutagenic potential. The substance was tested at 3000, 300, 30 and 3 μg/plate for possible activity to induce reversion of mutations at the histidine loci of two His-auxotrophic strains of Salmonella typhimurium: TA98 and TA100. Reverse mutation of the strains to histidine (His+) prototrophy is detected as growth on minimal medium that is deficient for histidine. In this assay, induction of reversion (positive mutagenicity) is indicated by 3-fold increase in the reversion frequency of compound treated groups compared to the spontaneous reversion of the vehicle control group. Colonies counts ≤50% of the vehicle control indicated cytotoxicity. The results are summarized in Table 6.

TABLE 6
Summary of Mutagenic Potential

| | | TA98 | |
|---|---|---|---|
| Treatment | Concentration | With S9 | Without S9 |

| | | | | | | |
|---|---|---|---|---|---|---|
| compound 17 | 3000 | μg/plate | + | − | − | − |
| | 300 | μg/plate | − | − | − | − |
| | 30 | μg/plate | − | − | − | − |
| | 3 | μg/plate | − | − | − | − |

| | | TA100 | |
|---|---|---|---|
| Treatment | Concentration | With S9 | Without S9 |

| | | | | | | |
|---|---|---|---|---|---|---|
| compound 17 | 3000 | μg/plate | − | − | − | − |
| | 300 | μg/plate | − | − | − | − |
| | 30 | μg/plate | − | − | − | − |
| | 3 | μg/plate | − | − | − | − |

−: No significant mutagenicity or cytotoxicity
+: Significant mutagenicity or cytotoxicity

### 1. Test Substance and Dosing Pattern

Test substance, compound 17, was dissolved in DMSO and serially diluted in ten-fold steps before adding to the top agar medium. The final concentrations of the test substances were 3000, 300, 30 and 3 μg/plate. The formulations are summarized in Table 7.

TABLE 7
Formulations of test compound

| Test | | | | Light | | Formulation |
|---|---|---|---|---|---|---|
| Compound | Vehicle | Solubility | Color | Protection | Temperature | (mg/mL) |
| Cmpd 17 | 100% | slightly | light | Formulation is | Prepared fresh | 30, 3, 0.3 and |
| | DMSO | soluble | yellow | kept in tube or | and stored | 0.03 |
| | | | | vial with | between 20-25° C. | |
| | | | | brown color, | | |
| | | | | or covered | | |
| | | | | with | | |
| | | | | aluminum foil | | |

### 2. Organisms

Two Salmonella typhimurium strains (TA98 and TA100) were obtained from Dr. Bruce N. Ames, University of California, Berkeley, USA as summarized in Table 8.

TABLE 8
Salmonella typhimurium strains

| Strain | Species | Genotype | Reverse mutation |
|---|---|---|---|
| TA 98 | S. typhimurium | hisD3052 rfa ΔuvrB- | Frame shift |
| | | gal pKM101 | |
| TA100 | S. typhimurium | hisG46 rfa ΔuvrB- | Base-pair substitution |
| | | gal pKM101 | |

### 3. Chemicals

2-Aminoanthracene (2-Anthramine) (Sigma, USA), Aroclor 1254-induced male Sprague Dawley rat liver S9 (Molecular Tox., Cat#11-01L.2, USA), β-Nicotinamide adenine dinucleotide phosphate (NADP, Sigma, USA), D-Biotin (Sigma, USA), Dimethyl sulfoxide (Merck, Germany), Glucose-6-phosphate (Merck, Germany), Glucose (Merck, Germany), L-Histidine HCl (Sigma, USA), Magnesium chloride (Wako, Japan), 4-Nitro-o-phenylenediamine (Sigma, USA), Potassium phosphate dibasic (Wako, Japan), Potassium chloride (Wako, Japan), Sodium azide (Sigma, USA), Sodium dihydrogen phosphate (Wako, Japan) and Sodium chloride (Wako, Japan).

### 4. Media

Bacto agar (DIFCO, USA), Bottom agar (containing 1.5% Bacto agar, 2% glucose and 2% vogel-bonner salt), Nutrient broth (Oxoid, England) and Top agar (0.6% Bacto agar and 0.5% NaCl supplemented with 0.05 mM histidine/0.05 mM biotin).

### 5. Equipment

Biological safety cabinet (NuAire, USA), Orbital shaking incubator (Firstek Scientific, Taiwan), Petri dishes (Gelman, USA), pH meter (SunTex, Taiwan), Pipetman (Rainin, USA) and Ultra-low temperature freezer (NuAire, USA).

### 6. Assays # [671200-671300] Ames Test, Salmonella Mutagenicity

Two histidine auxotrophic mutants (TA98 and TA100) of Salmonella typhimurium were used. Test strains were obtained from the frozen working stock and thawed at room temperature. A 0.2 mL aliquot was inoculated into 25 mL nutrient broth medium and then incubated at 35-37° C. with shaking (120 rpm) for 16-18 hr. Test substance was dissolved in DMSO with 10-fold dilutions to obtain 4 stock concentrations at 30,000, 3,000, 300 and 30 μg/mL. Rat liver microsome enzyme homogenate (S9) mixture was prepared containing 8 mM MgCl₂, 33 mM KCl, 4 mM NADP, 5 mM glucose-6-phosphate, 100 mM NaH₂PO₄ (pH 7.4) and 4% (v/v) Aroclor 1254-induced male rat liver microsome enzyme homogenate (S9). A 0.1 mL aliquot of test substance stock solution was combined with 0.1 mL strain culture and with 0.5 mL rat liver enzyme homogenate (S9) mixture or 0.5 mL PBS, and then the mixture was incubated at 35-37° C. with shaking (120 rpm) for 20 min. Molten top agar (2 mL containing 0.05 mM histidine and 0.05 mM biotin) was added, and then the mixture was poured onto the surface of a minimal glucose agar plate (30 mL of bottom agar per petri plate) to obtain final test concentrations at 3000, 300, 30 and 3 μg/plate. The plates were incubated at 37° C. for 72 hours, and then the numbers of His+ revertant colonies were counted. Treatments resulting in a three-fold increase (≥3×) in revertant colonies compared to the vehicle control were considered mutagenic. Treatments that reduce the colony counts to ≤50% of the vehicle control were considered cytotoxic. Assays were performed in triplicate. The individual plate count results are summarized in Tables 9 and 1.

TABLE 9
Results of Ames Test Assay, Salmonella Mutagenicity (Individual Plate Count)

| | | | Revertant Colonies/Plate, Mean ± SD, (n = 3) | | | | | | | |
|---|---|---|---|---|---|---|---|---|---|---|
| | | | Salmonella Mutagenicity | | | | Salmonella Mutagenicity | | | |
| | | | (TA98) | | | | (TA100) | | | |
| Treatment | Conc. | S9 | Individual | | | Mean ± SD | Individual | | | Mean ± SD |
| Spontaneous | — | (+) | 45 | 46 | 37 | 43 ± 5 | 344 | 324 | 318 | 329 ± 14 |
| Revertant | | | | | | | | | | |
| DMSO (Solvent) | 100 μL/plate | (+) | 39 | 34 | 39 | 37 ± 3 | 244 | 318 | 276 | 279 ± 37 |
| 2-Anthramine | 3 μg/plate | (+) | 7616 | 6096 | 7648 | 7120 ± 887* | 6432 | 7296 | 6288 | 6672 ± 545* |
| PT# 1200196 | 3000 μg/plate | (+) | 558 | 504 | 568 | 543 ± 34* | 356 | 284 | 316 | 319 ± 36 |
| Cmpd 17 | | | | | | | | | | |
| | 300 μg/plate | (+) | 118 | 106 | 96 | 107 ± 11 | 308 | 316 | 268 | 297 ± 26 |
| | 30 μg/plate | (+) | 55 | 63 | 48 | 55 ± 8 | 300 | 320 | 288 | 303 ± 16 |
| | 3 μg/plate | (+) | 40 | 43 | 42 | 42 ± 2 | 312 | 296 | 320 | 309 ± 12 |

Note:
Significant mutagenic activity (≥3× of control) denotes as (*);
S9 (+): Presence of S9. Number of revertants/plate was shown for each individual plate.
Mean ± SD value for triplcate plates of each treatment was calculated and shown behind the individual plate counts.

TABLE 10
Results of Ames Test, Salmonella Mutagenicity (Individual Plate Count)

| | | | Revertant Colonies/Plate, Mean ± SD, (n = 3) | | | | | | | |
|---|---|---|---|---|---|---|---|---|---|---|
| | | | Salmonella Mutagenicity | | | | Salmonella Mutagenicity | | | |
| | | | (TA98) | | | | (TA100) | | | |
| Treatment | Conc. | S9 | Individual | | | Mean ± SD | Individual | | | Mean ± SD |
| Spontaneous | — | (−) | 25 | 35 | 28 | 29 ± 5 | 296 | 307 | 302 | 302 ± 6 |
| revertant | | | | | | | | | | |
| DMSO (Solvent) | 100 μl/plate | (−) | 30 | 32 | 38 | 33 ± 4 | 270 | 272 | 260 | 267 ± 6 |
| 4-NPD | 30 μg/plate | (−) | 1128 | 1344 | 960 | 1144 ± 192* | — | — | — | — |
| Sodium Azide | 3 μg/plate | (−) | — | — | — | — | 1160 | 1120 | 944 | 1075 ± 115* |
| PT# 1200196 | 3000 μg/plate | (−) | 63 | 45 | 47 | 52 ± 10 | 222 | 218 | 234 | 225 ± 8 |
| Cmpd 17 | 300 μg/plate | (−) | 31 | 34 | 23 | 29 ± 6 | 314 | 310 | 244 | 289 ± 39 |
| | 30 μg/plate | (−) | 49 | 31 | 33 | 38 ± 10 | 308 | 262 | 272 | 281 ± 24 |
| | 3 μg/plate | (−) | 27 | 23 | 27 | 26 ± 2 | 202 | 150 | 144 | 165 ± 32 |

Note:
Significant mutagenic activity (≥3× of control) denotes as (*);
DMSO: Dimethyl Sulfoxide;
S9 (−): Absence of S9;
4-NPD: 4-Nitro-o-phenylenediamine;
—: No assay. Number of revertants/plate was shown for each individual plate.
Mean ± SD value for triplicate plates of each treatment was calculated and shown behind the individual plate counts.

### REFERENCES

1. Mortelmans K, Zeiger E. The Ames Salmonella/microsome mutagenicity assay. Mutat Res. 2000 Nov. 20; 455(1-2):29-60.
2. Maron, D. M. and Ames, B. N. Revised methods for the Salmonella mutagenicity test.Mutat. Res. 113: 173-215, 1983.
3. Levin, D. E., Yamasaki, E. and Ames, B. N. A new Salmonella tester strain, TA97, for the detection of frameshift mutagens. A run of cytosines as a mutational hot-spot. Mutat. Res. 94: 315-330, 1982.
4. Venitt S., Crofton-Sleigh C. and Forster R. Bacterial mutation assays using reverse mutation. In Mutagenecity testing: a practical approach. Oxford: IRL Press, 1984: 45-98.
5. James M. Parry and Elizabeth M. Parry (eds.), Genetic Toxicology: principles and methods, Methods in molecular biology, vol. 817. New York: Humana Press, c2012.

### Example 4—Tissue Distribution of Compounds after Oral Administration in Rat

The tissue distribution of compounds 17, 20, and 25 was assessed in the plasma and brain of Male SD rates. The compounds were delivered in a dose of 10 mg/kg in a vehicle comprising 10% Tween80 in 5% MC. The results are summarized in Table 11.

TABLE 11
Assessment of Plasma/Brain Levels after PO dosing

| Animal # | Time (hr) | Plasma (ng/mL) | Brain (ng/g) | B:P Ratio |
|---|---|---|---|---|
| | Compound 17 | | | |
| A1 | 1 | 16.2 | 7.78 | 0.48 |
| A2 | 1 | 47.0 | 15.5 | 0.33 |
| | Compound 25 | | | |
| B1 | 1 | 36.6 | 11.2 | 0.31 |
| B2 | 1 | 18.6 | 11.0 | 0.59 |
| | Compound 20 | | | |
| C1 | 1 | 70.2 | 30.6 | 0.44 |
| C2 | 1 | 101 | 41.5 | 0.41 |

### Example 5—In Vitro Metabolism of Compound 17 in Rat and Human Liver Microsomes

Preliminary assays were performed to tentatively identify metabolites of Compound 17 in Rat and Human Liver Microsomes following 10 μM incubation for 60 minutes. The results are summarized in Table 12.

TABLE 12
Metabolites of Compound 17

| | Tentative | Ion | Retention | | |
|---|---|---|---|---|---|
| Peak | Metabolite | Found | Time | | Species/ |
| ID | Identification | m/z | (min) | Product Ions | Matrix |
| Cmpd | Parent (P) | 456 | 7.36 | 439, 413, 399, | RLM, |
| 17 | | | | 386, 371, 359 | HLM |
| A | P + O | 472 | 7.66 | 454, 413, 399, | RLM, |
| | | | | 385, 371 | HLM |
| B | P + O − 2 H | 470 | 6.64 | 415, 399 | RLM, |
| | | | | | HLM |
| C | P + O + O | 488 | 4.19 | 470, 442, 430, | RLM |
| | | | | 412, 399, 386, | |
| | | | | 136 | |
| D | P + O + O − | 486 | 5.64 | 415, 386 | RLM, |
| | 2 H | | | | HLM |

Briefly, oxidation appears to be centralized on the piperazine ring of Compound 17. Metabolite B was the major component observed in the rat liver microsome incubation (with NADPH) based on MS and UV peak intensities. Parent and metabolites A and B were the major components observed in human. Metabolite C was not observed in human. Metabolites A and B were observed in the rat and human incubations without NADPH, however they were minor.

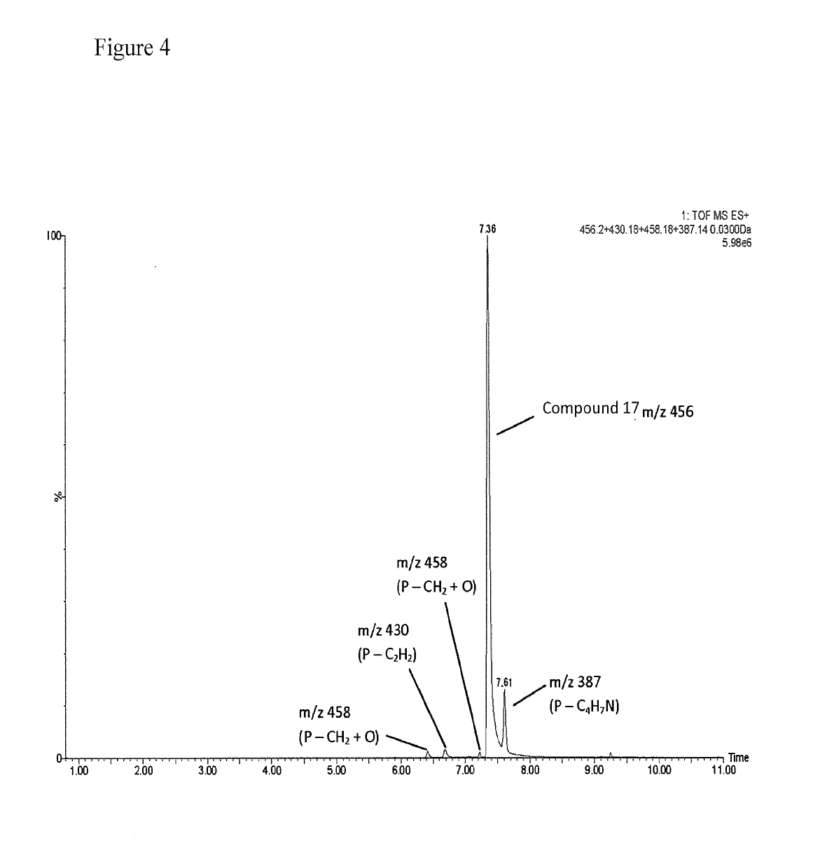

Several impurities were observed in the neat standard of Compound 17. An extracted ion chromatogram of the neat standard of Compound 17 is shown in FIG. 4. The impurity masses were not used to produce the extracted ion chromatograms for metabolite profiling as there was no obvious increase in MS peak intensities for the impurities between the without and with NADPH samples. The impurity for m/z 458 was observed in the UV chromatograms for both rat and human.

Instrumentation—

UPLC System: Waters Acquity System (SN's: D12USM306G, G12CPO360N, D12BUR530M, E12CMP754G) Mass Spectrometer: Waters Synapt G2-S(SN UEB102).

UPLC Conditions—

Mobile Phase A: 10 mM Ammonium Bicarbonate; Mobile Phase B: Acetonitrile; Gradient (Minutes/% B): 0/5, 0.5/5, 8/45, 9/95, 10.25/95, 10.5/5, 12/stop; HPLC Column: Waters Acquity BEH C18, 2.1×100 mm, 1.7 μm particle size. Column Temp: 60° C. Flow Rate

(mL/min): 0.5

### Injection Volume (4): 5

### Divert: 0.8 min

Mass Spectrometry Conditions—

Ionization: Positive ESI

### Capillary: 1.0 kV

Sample cone: 30 V

### Source Temperature: 120° C. MS/MS Collision Energy: 25 V

### Desolvation Temperature: 500° C. Acquired TOF MS Range: 100 to 1000 Da

Sample Preparation—

Compound 17: The microsomal incubations were prepared by adding cryopreserved liver microsomes to 100 mM sodium phosphate buffer (pH 7.4) to give a final incubated protein concentration of 1 mg/mL. The stock solution of Compound 17 (10 mM in DMSO) was diluted in phosphate buffer to 0.2 mM and then added to the microsomes to provide a final incubated concentration of 10 μM. Verapamil was used as a positive control in each species. The test article and positive control were incubated with and without NADPH (2 mM final concentration) for 60 minutes at 37° C. after which reactions were quenched 1:1 with acetonitrile. Samples were vortexed and centrifuged at 5,000 rpm for 15 minutes to remove proteins. Supernatants were analyzed as received.

The verapamil controls were analyzed along with the samples. Acceptance of the microsomal incubations is based on the qualitative formation of Phase I metabolites in the verapamil control samples for each species.

LC-MS/UV raw data files and the Discovery Biotransformation Summary Reports are archived electronically at Q Squared Solutions.

Rat liver microsomes: Gentest, lot 4220006 (210 male donors)

Human liver microsomes: Xenotech, lot 1410230 (100 male and 100 female donors)

### Example 6—Cardiac Profile for Compounds of Formula I

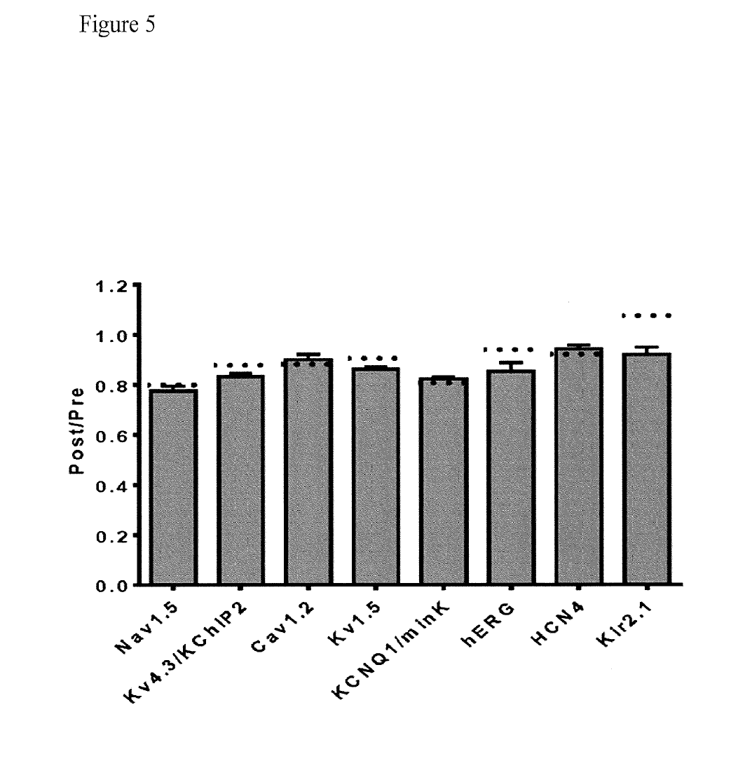

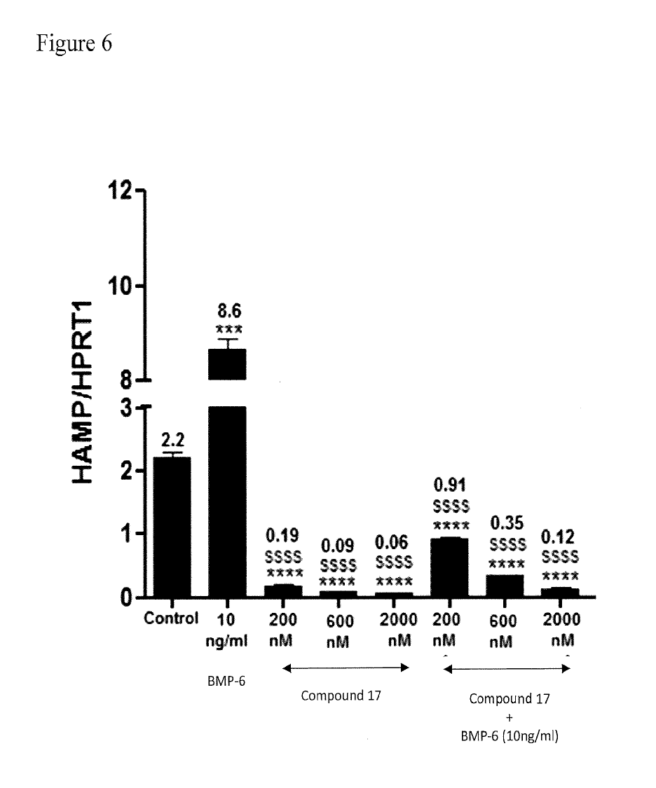

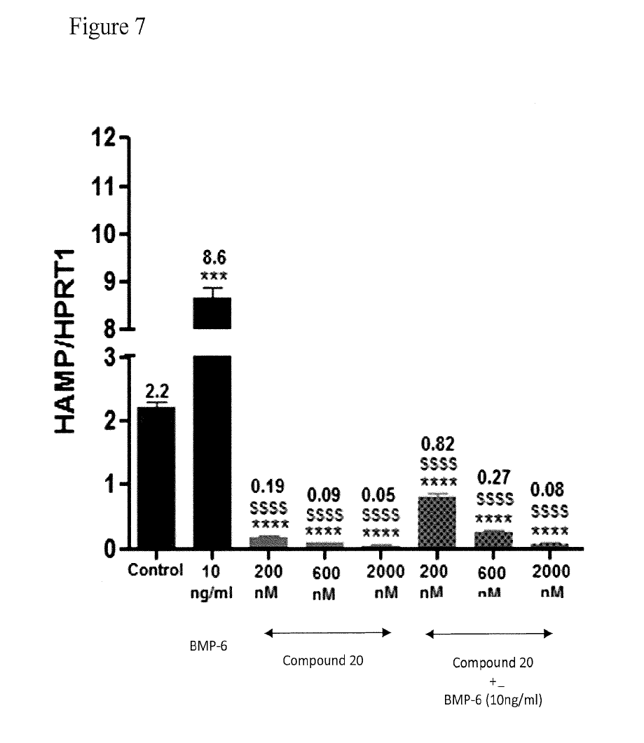

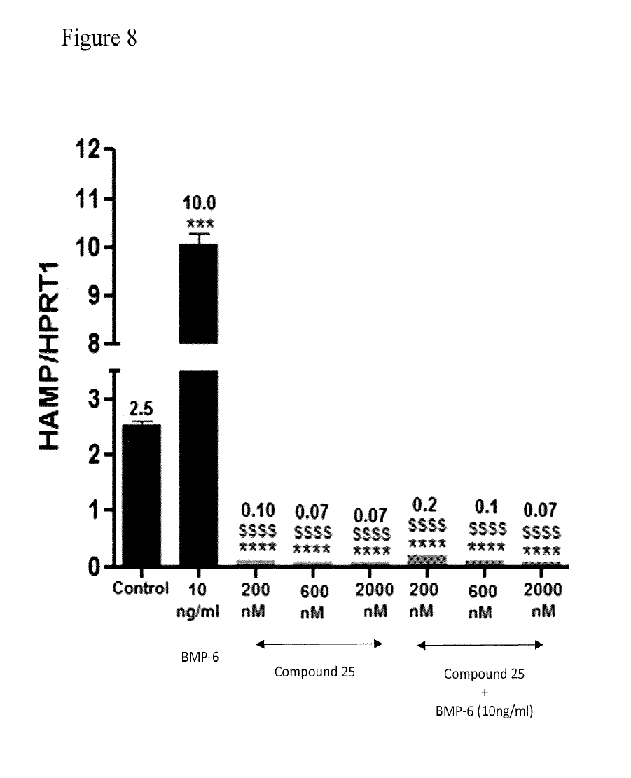

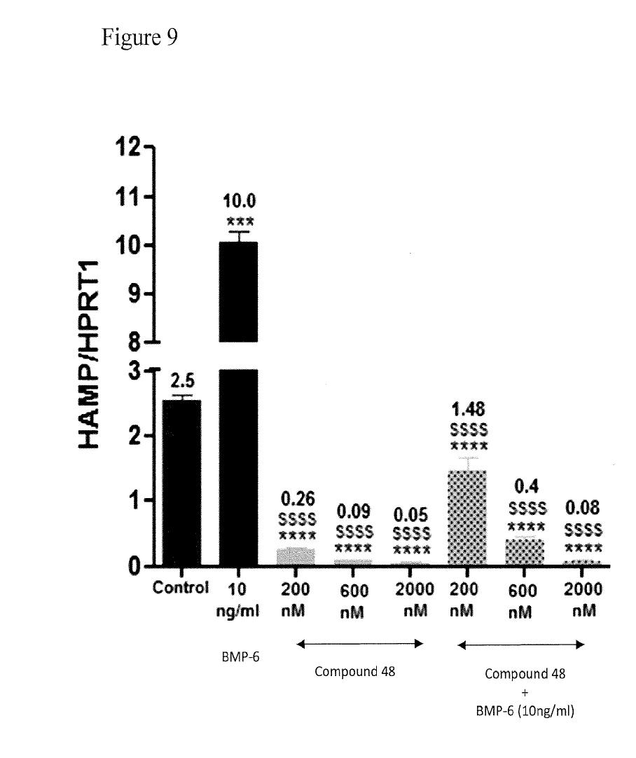

To gain an insight into the potential cardiac risks for certain compounds of the invention, the effects of compound 17 on profiled ion channels were assayed. The results are shown in FIG. 5 and Table 13 below. The parameters measured for each cell line are described in the data analysis sections below. The dashed horizontal line associated with each bar indicates the mean effect of vehicle control wells (0.3% DMSO). For those channels where two parameters were measured, the data and DMSO control line shown in FIG. 5 represent the data from the second parameter.

TABLE 13
Cardiac Profiler Panel

| | % Inhibition | | | | | | | |
|---|---|---|---|---|---|---|---|---|
| | Nav1.5 | Kv4.3 | Cav1.2 | Kv1.5 | KCNQ | hERG | HCN4 | Kir2.1 |
| Cmpd 17 | 20.2 | 16.6 | 10.3 | 14.8 | 17.7 | 14.6 | 5.6 | 7.7 |

### Materials and Methods

Compound 17 was prepared as a 10 mM stock solution in DMSO and was further diluted to 300× the final assay concentration of 10 μM. All 300× DMSO stock solution was transferred to a master plate and into assay plates where 2 μl per well of each 300× solution were placed. All assay plates were stored at −80° C. until the day of assay.

On the day of the assay, the appropriate assay plate was thawed at room temperature, centrifuged, and 198p1 of external solution was added and mixed thoroughly. This provided a 1:100 dilution. A further 1:3 dilution occurred upon addition to the cells in the IonWorks, giving a 1:300 dilution in total.

On each assay plate, at least 8 wells were reserved for vehicle control (0.3% DMSO) and at least 8 wells for each positive control specific to the cell line tested. The positive controls were tested at a maximal blocking and an approximate IC₅₀ concentration. The positive control compounds are outlined in Table 14.

TABLE 14
Controls for Cardiac Profile

| Ion Channel | Positive Control & Concentrations |
|---|---|
| Nav1.5 | 100 μM and 5 mM Lidocaine |
| Kv4.3/KChIP2 | 20 μM and 500 μM Quinidine |
| Cav1.2 | 50 μM and 333 μM Verapamil |
| Kv1.5 | 300 μM and 10 mM 4-AP |
| KCNQ1/minK | 10 μM and 100 μM Chromanol 293B |
| hERG | 0.1 μM and 1 μM Cisapride |
| HCN4 | 50 μM and 3 mM Cesium Chloride |
| Kir2.1 | 20 μM and 500 μM Barium |

The solutions for recording potassium currents (e.g. hERG, Kv1.5, Kv4.3/KChIP2, KCNQ1/minK, Kir2.1) were as follows: External Recording Solution (NaGluconate 130 mM, NaCl 20 mM, KCl 4 mM, MgCl₂ 1 mM, CaCl₂ 1.8 mM, HEPES 10 mM, glucose 5 mM, pH 7.3 (titrated with 10 M NaOH)) and Internal Recording Solution (K gluconate 100 mM, KCl 40 mM, MgCl₂ 1 mM, HEPES 10 mM, EGTA 1 mM, pH 7.3 (titrated with 10 M NaOH)).

The solutions for recording HCN4 currents were as follows: External Recording Solution (NaGluconate 104 mM, NaCl 10 mM, KCl 30 mM, MgCl₂ 1 mM, CaCl₂ 1.8 mM, HEPES 10 mM, glucose 5 mM, pH 7.3 (titrated with 10M NaOH)) and Internal Recording Solution (K Gluconate 130 mM, NaCl 10 mM, MgCl₂ 1 mM, HEPES 10 mM, EGTA 1 mM, pH 7.3 (titrated with 10M KOH)).

The solutions for recording Nav1.5 currents were as follows: External Recording Solution (NaCl 137 mM, KCl 4 mM, MgCl₂ 1 mM, CaCl₂ 1.8 mM, HEPES 10 mM, glucose 5 mM, pH 7.3 (titrated with 10M NaOH)) and Internal Recording Solution (CsF 90 mM, CsCl 45 mM, HEPES 10 mM, EGTA 1 mM, pH 7.3 (titrated with 1M CsOH)).

The solutions for recording Cav 1.2 currents were as follows: External Recording Solution (NaGluconate 130 mM, NaCl 20 mM, KCl 5 mM, MgCl₂ 2 mM, BaCl₂ 10 mM, HEPES 10 mM, glucose 5 mM, pH 7.3 (titrated with 10M NaOH)) and Internal Recording Solution (K Gluconate 100 mM, CsCl 40 mM, MgCl₂ 0.2 mM, HEPES 10 mM, EGTA 1 mM, pH 7.3 (titrated with 1M CsOH), osmolality adjusted with sucrose).

Amphotericin B was used to obtain electrical access to the cell interior at a final concentration of 200 μg/ml in internal recording solution.

### Experimental Protocols & Data Analysis

Nav1.5 Experimental Protocol—Human Nav1.5 currents were repetitively evoked by stepping from a holding potential of −120 mV to −20 mV for 150 ms (50 ms inter-pulse interval) for a total of 26 pulses. The voltage protocol was applied (Pre), compounds added, incubated for 600 seconds, and the voltage protocol was applied a final time (Post) on the IonWorks Quattro.

Nav1.5 Data Analysis—The parameters measured were the maximum inward current evoked on stepping to −20 mV from the 1st and 26th pulse. All data were filtered for seal quality, seal drop, and current amplitude. The peak current amplitude (Peak) was calculated before (Pre) and after (Post) compound addition and the amount of block was assessed by dividing the Post-compound current amplitude by the Pre-compound current amplitude. These procedures were implemented for the 1st and 26th pulse.

Kv4.3/KChIP2 Experimental Protocol—Human Kv4.3/KChIP2 currents were evoked from a holding potential of −80 mV by a series of four 500 ms pulses to 0 mV using a 1000 ms interval between pulses. The voltage protocol was applied (Pre), compounds added, incubated for 600 seconds, and the voltage protocol was applied a final time (Post) on the IonWorks Quattro.

Kv4.3/KChIP2 Data Analysis—The parameter measured was the amplitude of the outward current at a time point of 50 ms after the onset of the 4th voltage step to 0 mV. All data were filtered for seal quality, seal drop, and current amplitude. The current amplitude of the 4th pulse was calculated before (Pre) and after (Post) compound addition and the amount of block assessed by dividing the Post-compound current amplitude by the Pre-compound current amplitude.

Cav1.2 Experimental Protocol—Human Cav1.2 currents were evoked by two pulses to −10 mV from a holding potential of −100 mV. The duration of the first pulse at −10 mV is 500 ms and the 2nd pulse is 100 ms. The voltage protocol was applied (Pre), compounds added, incubated for 292 seconds, and the voltage protocol was applied a final time (Post) on the IonWorks Quattro.

Cav1.2 Data Analysis—The parameters measured were the maximum inward currents evoked on stepping to −10 mV from the holding potential of −100 mV for the 1st and 2nd pulse. All data were filtered for seal quality, seal drop, and current amplitude. The peak current amplitude (Peak) was calculated before (Pre) and after (Post) compound addition and the amount of block was assessed by dividing the Post-compound current amplitude by the Pre-compound current amplitude. These procedures were implemented for the 1st and 2nd pulse.

Kv1.5 Experimental Protocol—Human Kv1.5 currents were evoked by a single pulse from a holding potential of −80 mV to a potential of 0 mV for a period of four seconds before returning to −80 mV. The voltage protocol was applied (Pre), compounds added, incubated for 600 seconds, and the voltage protocol was applied a final time (Post) on the IonWorks Quattro.

Kv1.5 Data Analysis—The parameters measured were the maximum amplitude of outward currents evoked at the beginning of the voltage pulse (Peak) and at the end of the voltage step from −80 mV to 0 mV (End). All data were filtered for seal quality, seal drop, and current amplitude. The current amplitudes (Peak & End) were calculated before (Pre) and after (Post) compound addition and the amount of block was assessed by dividing the Post-compound current amplitude by the Pre-compound current amplitude. These procedures were implemented for the peak and end of the single depolarizing pulse to 0 mV.

KCNQ1/minK Experimental Protocol—Human KCNQ1/minK currents were evoked by a single pulse delivered from a holding potential of −80 mV to +60 mV for four seconds before returning to −80 mV. The voltage protocol was applied (Pre), compounds added, incubated for 600 seconds, and the voltage protocol was applied a final time (Post) on the IonWorks Quattro.

KCNQ1/minK Data Analysis—The parameter measured was the maximum outward current evoked on stepping to +60 mV from the holding potential of −80 mV. All data were filtered for seal quality, seal drop, and current amplitude. The maximum current amplitude of the single pulse was calculated before (Pre) and after (Post) compound addition and the amount of block assessed by dividing the Post-compound current amplitude by the Pre-compound current amplitude.

hERG Experimental Protocol—hERG currents were evoked by a three pulse protocol where voltage was first stepped to +40 mV for two seconds from a holding potential of −80 mV to inactivate hERG channels. The voltage is then stepped back to −50 mV for two seconds to evoke a tail current prior to returning to the holding potential for 1 second. The voltage protocol was applied (Pre), compounds added, incubated for 300 seconds, and the voltage protocol was applied a final time (Post) on the IonWorks Quattro.

hERG Data Analysis—The parameter measured was the amplitude of the 3rd pulse tail current upon stepping back to −50 mV after the step to +40 mV. All data were filtered for seal quality, seal drop, and current amplitude. The maximum current amplitude of the 3rd pulse tail current was calculated before (Pre) and after (Post) compound addition and the amount of block assessed by dividing the Post-compound current amplitude by the Pre-compound current amplitude.

HCN4 Experimental Protocol—Human HCN4 currents were evoked by a single pulse from a holding potential of −30 mV to a potential of −110 mV for a duration of four seconds prior to returning to −30 mV. The voltage protocol was applied (Pre), compounds added, incubated for 600 seconds, and the voltage protocol was applied a final time (Post) on the IonWorks Quattro.

HCN4 Data Analysis—The parameter measured was the maximum inward current evoked upon stepping to −110 mV from the holding potential of −30 mV. All data were filtered for seal quality, seal drop, and current amplitude. The maximum current amplitude of the single hyperpolarizing pulse was calculated before (Pre) and after (Post) compound addition and the amount of block assessed by dividing the Post-compound current amplitude by the Pre-compound current amplitude.

Kir2.1 Experimental Protocol—Human Kir2.1 currents were evoked from a holding potential of −20 mV by a series of ten 500 ms pulses to −120 mV using a 200 ms interval between pulses. The voltage protocol was applied (Pre), compounds added, incubated for 600 seconds, and the voltage protocol was applied a final time (Post) on the IonWorks Quattro.

Kir2.1 Data Analysis—The parameters measured were the amplitudes of the instantaneous inward currents evoked on stepping to −120 mV for the 1st pulse and the maximum inward current at the end of the 10th hyperpolarizing pulse. All data were filtered for seal quality, seal drop, and current amplitude. The peak current amplitude (P1ᵢₙᵢₜᵢₐₗ & P10_{end}) was calculated before (Pre) and after (Post) compound addition and the amount of block was assessed by dividing the Post-compound current amplitude by the Pre-compound current amplitude. These procedures were implemented for the 1st and 10th pulse

### Example 7—Additional Drug Metabolism and Pharmacokinetic Studies

Data from additional drug metabolism and pharmacokinetic studies are summarized in the below tables. Briefly, Tables 15 and 16 summarize the Cytochrome p450 inhibition data which suggest that CYP inhibition is moderate. Additionally, phenotyping shows mutltiple CYPs participate in metabolism. Tables 17 and 18 summarize the Metabolic Stability data in Microsomes and Hepatocytes. Tables 19 and 20 summarize the in vivo IV/PO cross-over data in rats showing that compounds of the invention have low-to-moderate clearance (CL<20 ml/min/kg), a t_{1/2}>5 h in rats, and good bioavailability (F>25%). Tables 21-23 summarize the in vivo dose escalation studies of orally (PO) administered compounds in rats. Tables 24 and 25 summarize the Plasma:Brain level studies and MDR1-MDCK permeability data.

TABLE 15
CYP inhibition data

| | | |
|---|---|---|
| Cmpd 17 | Cmpd 20 | Cmpd 25 |

CYP (% inh. @ 3 mM), NI—no inhibition

| | | | |
|---|---|---|---|
| 3A4 | 24 | NI | 1 |
| 2D6 | 6 | 11 | 19 |
| 2C9 | 10 | 7 | 15 |
| 2C19 | 7 | NI | 2 |
| 1A2 | NI | NI | NI |

CYP IC₅₀ (mM)

| | | | |
|---|---|---|---|
| 3A4 | 5.7 | 12.7 | 16.8 |
| 2D6 | >20 | >20 | >20 |
| 2C9 | >20 | >20 | >20 |

CYP Phenotyping (% metabolized in rCYPs)

| | | | |
|---|---|---|---|
| 3A4 | 21 | 27 | 37 |
| 2D6 | na | 30 | 18 |
| 2C9 | 19 | 19 | 39 |
| 2C19 | 15 | 25 | 34 |
| 1A2 | 11 | 30 | 47 |

TABLE 16
Percent contribution by isoenzymes in metabolism
of certain compounds of Formula I

| | Cmpd 17 | Cmpd 20 | Cmpd 25 |
|---|---|---|---|
| 3A4 | 67 | 64 | 58 |
| 2D6 | −1 | 3 | 1 |
| 2C9 | 21 | 16 | 20 |
| 2C19 | 4 | 4 | 6 |
| 1A2 | 7 | 13 | 15 |

TABLE 17
Metabolic Stability - Microsomes

| | | | | Cmpd 17 | | | Cmpd 20 | | | Cmpd 25 | | |
|---|---|---|---|---|---|---|---|---|---|---|---|---|
| Human | % | T_{1/2} | mCL_{INT} | 27 | 80 | 19 | 19 | 86 | 16 | 33 | 58 | 24 |
| | met | | | | | | | | | | | |
| Mouse | % | T_{1/2} | mCL_{INT} | 40 | 44 | 33 | 46 | 37 | 37 | 46 | 33 | 42 |
| | met | | | | | | | | | | | |
| Rat | % | T_{1/2} | mCL_{INT} | 53 | 30 | 47 | 61 | 23 | 61 | 65 | 21 | 66 |
| | met | | | | | | | | | | | |
| Dog | % | T_{1/2} | mCL_{INT} | 71 | 16 | 86 | 82 | 12 | 116 | 84 | 11 | 123 |
| | met | | | | | | | | | | | |
| Monkey | % | T_{1/2} | mCL_{INT} | 43 | 42 | 33 | 47 | 32 | 44 | 60 | 24 | 59 |
| | met | | | | | | | | | | | |

TABLE 18
Metabolic Stability - Hepatocytes

| | | |
|---|---|---|
| Cmpd 17 | Cmpd 20 | Cmpd 25 |

Metabolic Stability - Hepatocytes (% met. @ 180 min.;
CL_{HEP}, (mL/min/kg), T_{1/2}, (min))

| | | | | | | | | |
|---|---|---|---|---|---|---|---|---|
| Human | % met | T_{1/2} | 24 | 443.2 | 29.1 | 394.2 | 36.1 | 283.1 |
| CL_{HEP} | | CL blood | 9.7 | 0.46 | 10.3 | 0.49 | 12.0 | 0.57 |
| | | flow | | | | | | |
| Rat | % met | T_{1/2} | 22 | 530.8 | 32.2 | 343.6 | 22.1 | 443.6 |
| CL_{HEP} | | CL blood | 8.8 | 0.42 | 11.0 | 0.53 | 9.7 | 0.46 |
| | | flow | | | | | | |
| Mouse | % met | T_{1/2} | 21 | 582.5 | 21 | 527.9 | 30 | 384.1 |
| CL_{HEP} | | CL blood | 10.9 | 0.52 | 11.5 | 0.55 | 13.1 | 0.62 |
| | | flow | | | | | | |

TABLE 19
In vivo - Discrete IV PK (0.5 mg/kg) Crossover - Rat

| | Cmpd 17 | Cmpd 20 | Cmpd 25 |
|---|---|---|---|
| CL (mL/min/kg) | 16.7 ± 2.1 | 12.9 ± 3.7 | 17.1 ± 1.6 |
| T_{1/2} (h) | 5.4 ± 0.6 | 5.9 ± 3.5 | 5.9 ± 0.6 |
| Cₘₐₓ (ng/mL) | 83.6 ± 19.4 | 144 ± 5.7 | 133.2 ± 24.0 |
| C₀ (ng/mL) | 93.8 ± 23.3 | 151 ± 5.2 | 143.0 ± 21.7 |
| Vd (L/kg) | 7.7 ± 0.6 | 5.8 ± 1.2 | 8.8 ± 1.7 |
| Vdₛₛ (L/kg) | 6.8 ± 0.8 | 5.5 ± 1.2 | 7.3 ± 1.4 |
| AUC₀₋ₜ (hr*ng/mL) | 456.3 ± 99.6 | 435 ± 29.9 | 464.1 ± 48.5 |
| AUC_{0-∞} (hr*ng/mL) | 503.9 ± 59.4 | 701 ± 267.3 | 489.6 ± 45.5 |

TABLE 20
In vivo - Discrete oral PK (3 mg/kg) Crossover - Rat

| | Cmpd 17 | Cmpd 20 | Cmpd 25 |
|---|---|---|---|
| T_{1/2} (h) | 3.8 ± 0.4 | 3.9 ± 0.7 | 4.7 ± 0.5 |
| Cₘₐₓ (ng/mL) | 95.7 ± 41.5 | 163 ± 11.8 | 127.7 ± 53.3 |
| Tₘₐₓ (hr) | 4.00 | 4.00 | 4.00 |
| AUC₀₋ₜ (hr*ng/mL) | 905.4 ± 365.4 | 1637 ± 532 | 1235.9 ± 620.9 |
| AUC_{0-∞} (hr*ng/mL) | 920.5 ± 369.0 | 1683 ± 579 | 1289.0 ± 650.7 |
| % F | 29.8 ± 9.1 | 40 | 45.3 ± 26.2 |

TABLE 21
In vivo dose escalation - Rat (oral) - Cmpd 17

Cmpd 17
Dose (mg/Kg)

| PK Parameters | 3 | 10 | 30 | 100 |
|---|---|---|---|---|
| AUC₀₋ₜ (ng*h/mL) | 905 | 3126 | 14678 | 33706 |
| Cₘₐₓ (ng/mL) | 95 | 294 | 1392 | 3475 |
| Tₘₐₓ (h) | 4.0 | 4.0 | 5.0 | 4.0 |
| T_{1/2} (h) | 3.78 | 4.5 | 6.7 | 15.9 |
| % F | 30.7 | 32.2 | 54.3 | 51.2 |

TABLE 22
In vivo dose escalation - Rat (oral) - Cmpd 20

Cmpd 20
Dose (mg/Kg)

| PK Parameters | 3 | 10 | 30 | 100 |
|---|---|---|---|---|
| AUC₀₋ₜ (ng*h/mL) | 1637 | 6612 | 31370 | 58398 |
| Cₘₐₓ (ng/mL) | 163 | 652 | 2493 | 4153 |
| Tₘₐₓ (h) | 4.0 | 3.5 | 4.0 | 3.5 |
| T_{1/2} (h) | 3.9 | 4.8 | 6.3 | 9.5 |
| % F | 40 | 49 | 82 | 52 |

TABLE 23
In vivo dose escalation - Rat (oral) - Cmpd 25

Cmpd 25
Dose (mg/Kg)

| PK Parameters | 3 | 10 | 30 | 100 |
|---|---|---|---|---|
| AUC₀₋ₜ (ng*h/mL) | 1235 | 3955 | 20602 | 38633 |
| Cₘₐₓ (ng/mL) | 127 | 338 | 1735 | 2980 |
| Tₘₐₓ (h) | 4.0 | 5.0 | 5.0 | 2.5 |
| T_{1/2} (h) | 4.7 | 4.2 | 6.1 | 7.6 |
| % F | 45 | 42 | 76 | 46 |

TABLE 24
Plasma Brain Level Study Data

| Cmpd | | | | | | |
|---|---|---|---|---|---|---|
| 10 mg/kg | Plasma | Brain | B:P | Plasma | Brain | B:P |
| | 1 h, PO | | | 5 h, PO | | |
| 17 | 145 ± 53 | 41 ± 17 | 0.29 | 542 ± 252 | 320 ± 174 | 0.59 |
| 20 | 102 ± 85 | 163 ± 99 | 1.61 | 288 ± 28 | 1351 ± 210 | 4.68 |
| 25 | 240 ± 69 | 84 ± 8 | 0.35 | 575 ± 95 | 322 ± NA | 0.56 |
| | 1 h, IV | | | 5 h, IV | | |
| 17 | 602 ± 117 | 633 ± 413 | 1.05 | 297 ± 60 | 440 ± 177 | 1.48 |
| 20 | 773 ± 96 | 618 ± 146 | 0.8 | 484 ± 52 | 311 ± 47 | 0.64 |
| 25 | 682 ± 11 | 450 ± 295 | 0.66 | 367 ± 96 | 466 ± 210 | 1.27 |

TABLE 25
MDR1-MDCK Permeability Data

| | MDCK Permeability Pₐₚₚ (10⁻⁶ cm/s) | | |
|---|---|---|---|
| Compound | A-B | B-A | ER (B-A/A-B) |
| 17 | 2.2 | 9.1 | 4.14 |
| 20 | 2.54 | 8.4 | 3.31 |
| 25 | 2.41 | 10.84 | 4.5 |

### Example 8—Efficacy of Compounds in an In Vitro Model of Hepcidin Expression

The efficacy of compounds 17, 20, 25, and 48 was shown in an in vitro model of Hepcidin expression. Briefly, compounds 17, 20, 25, and 48 reduced Hepcidin expression as compared to control (See Figures, 6, 7, 8, and 9).

### Example 9—Efficacy of Compounds in an IRIDA Animal Model

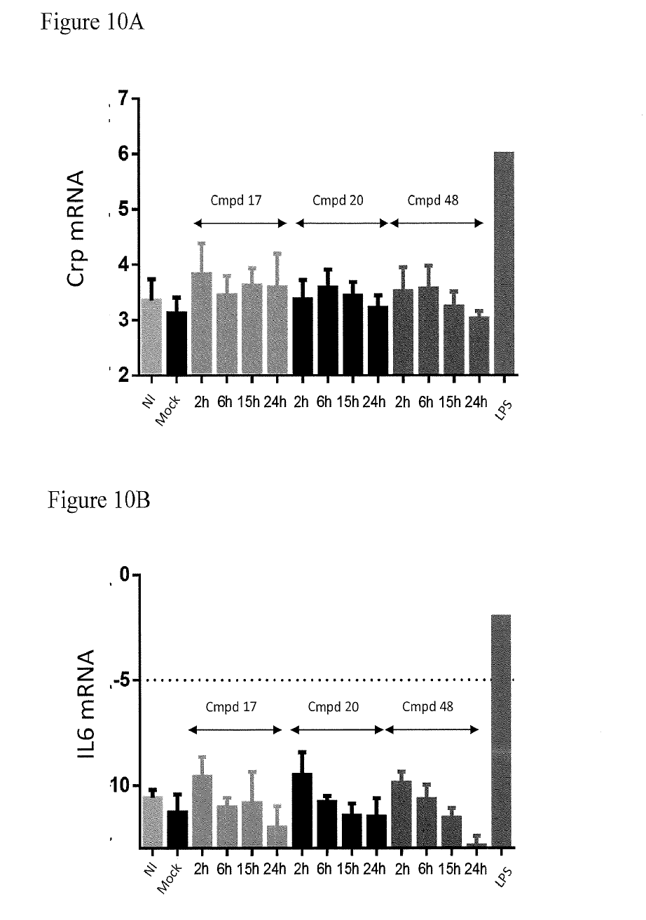

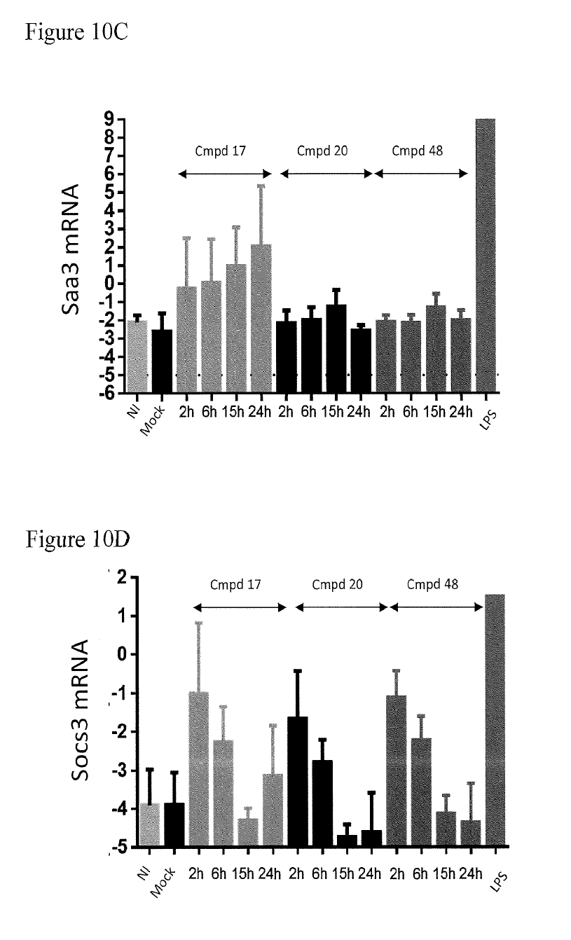

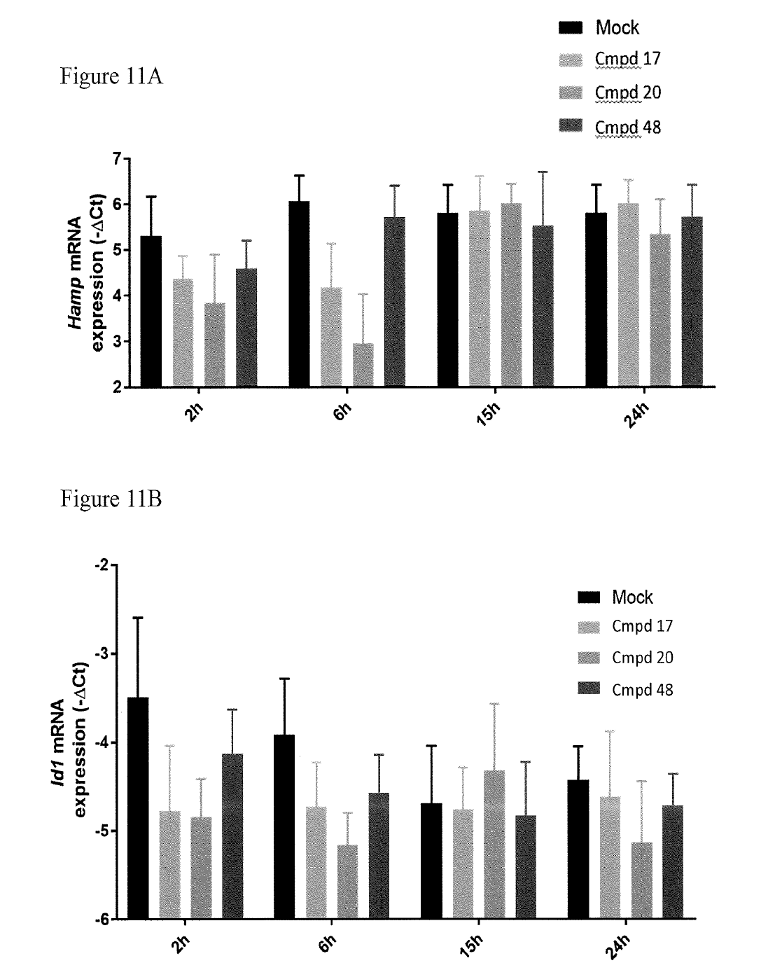

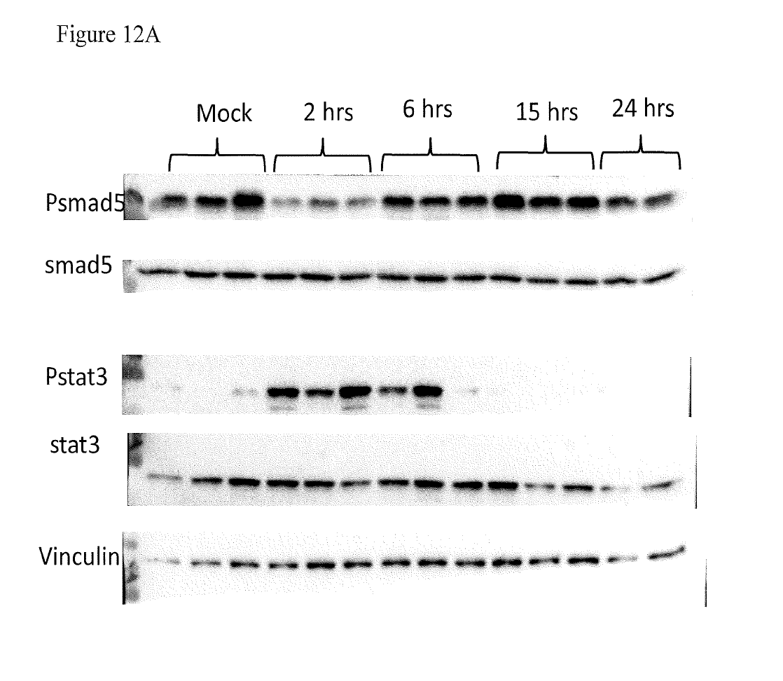

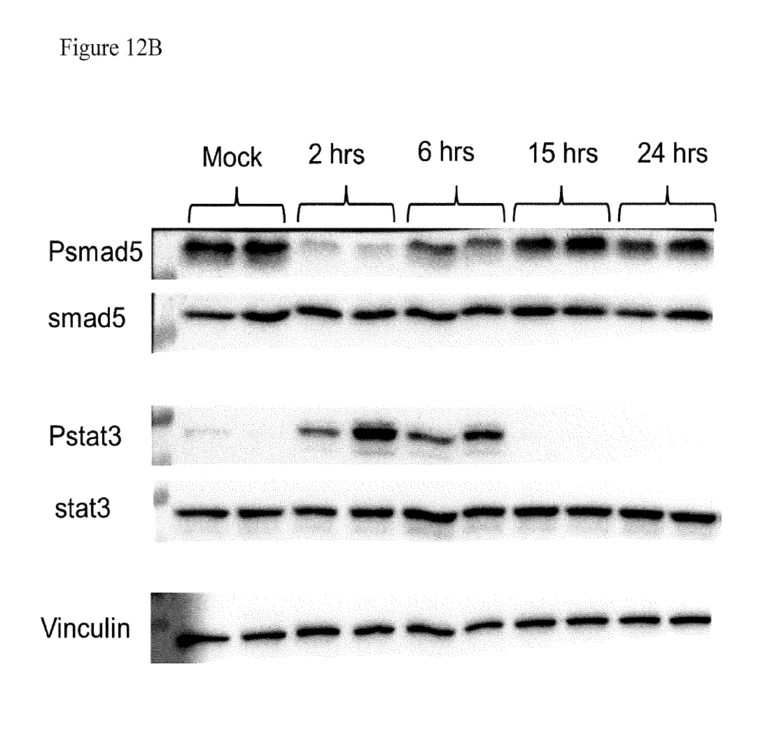

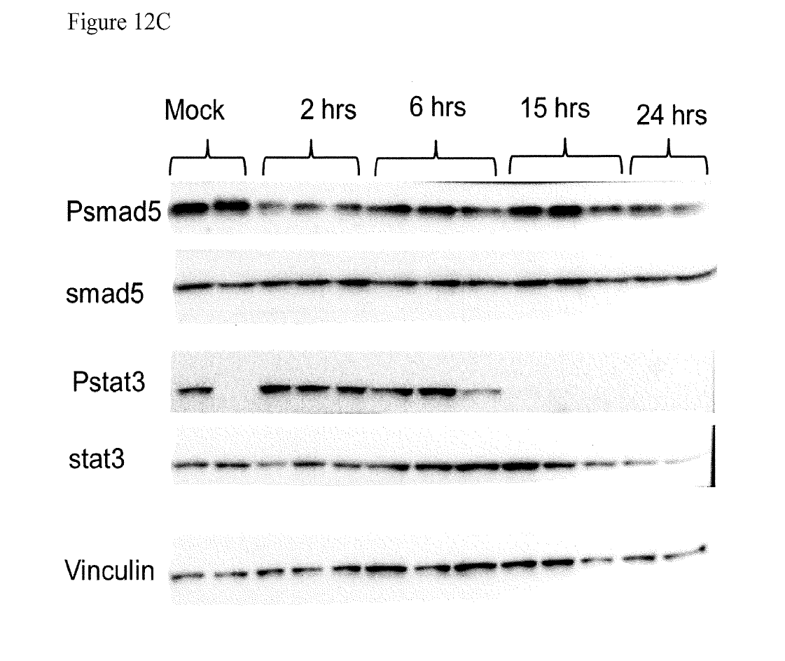

The efficacy of compounds was shown in an IRIDA animal model. Initially, an exemplary dose and time point was determined in wild type mice. Briefly, liver mRNA and proteins were extracted. Hepcidin and Id1 mRNA expression was measured by real-time PCR and Smad 1-5-8 phosphorylation was assayed by Western blot analysis. FIGS. 10A-10D depict the RT-PCR data for various inflammatory markers in the liver. FIGS. 11A and 11B depict the RT-PCR data for Hamp mRNA and Id1 mRNA expression. FIG. 12A-12C depict the results of the Western blot analysis.

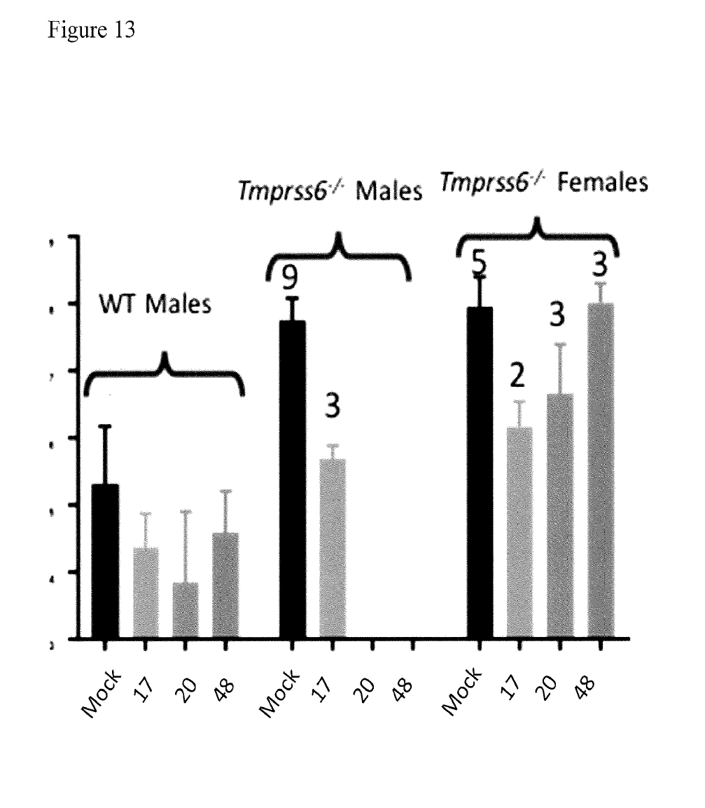

From the data obtained above, the reduction of hepcidin expression and SMAD signaling in Tmprss6−/− mice was demonstrated. FIG. 13 show Liver hepcidin mRNA expression levels 2 hours after injecting Tmprss6−/− mice with compounds 17, 20, or 48. The treatment did not affect the body weight or the global health of the Tmprss6−/− mice. Notably, daily injections of compound 20 improved the alopecia of the Tmprss6−/− mice suggesting an improvement in iron deficiency.

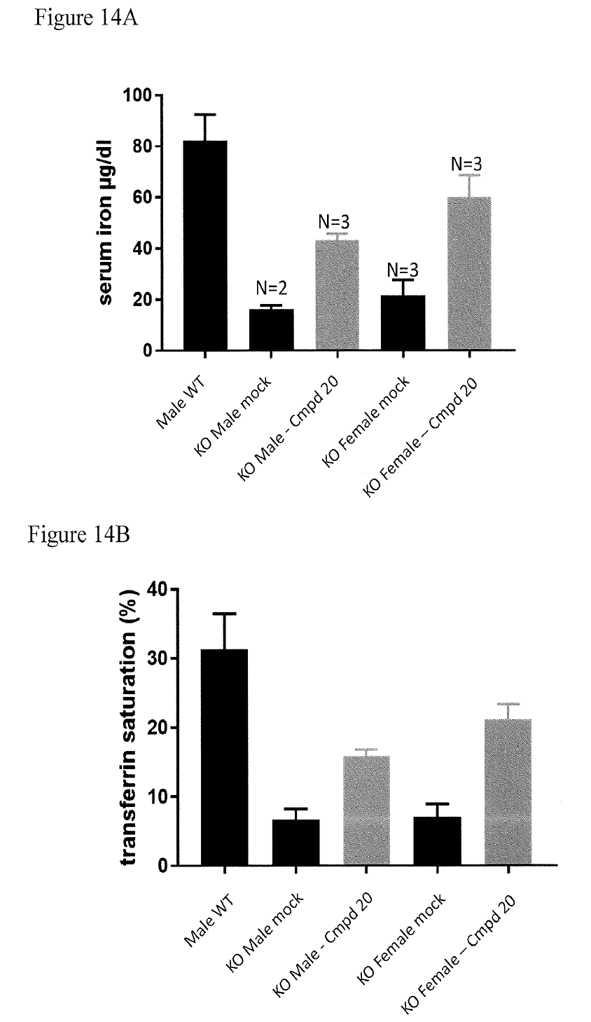

Further studies assayed whether iron deficiency improved after 4 or 7 weeks of treatment. The results after 4 weeks of treatment are summarized in FIGS. 14A and 14B. For the 7 week study, Tmprss6−/− mice were treated with compound 20 by two IP injections (8 a.m. and 5:30 p.m.) at a dose of 20 mg/kg in 100 μl. Mock treated mice similarly received two IP injections per day of 100 μl of (2-hydroxypropyl)-B-cyclodextrin solution. Mice were sacrificed 6 hours after the last injection. For each group, n=5. The treatment did not affect the body weight of the mice or their overall global health. Notably, compound 20 injected twice a day for 7 weeks improved the Tmprss6−/− mice alopecia suggesting an improvement of iron deficiency.

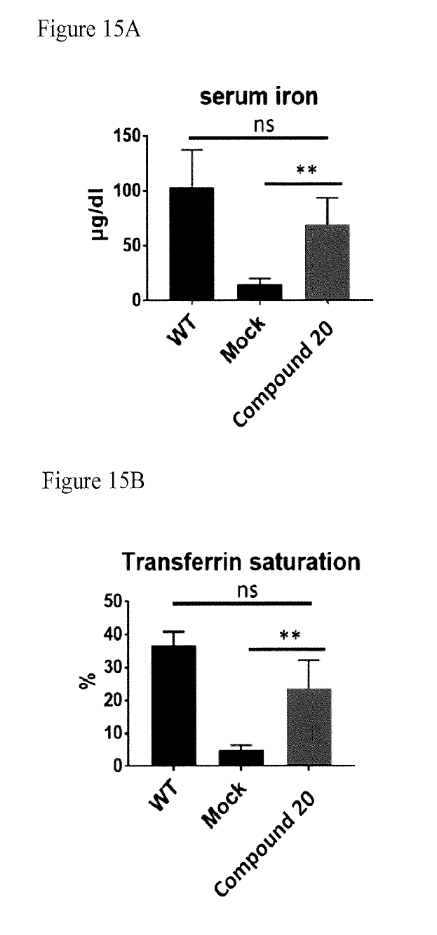

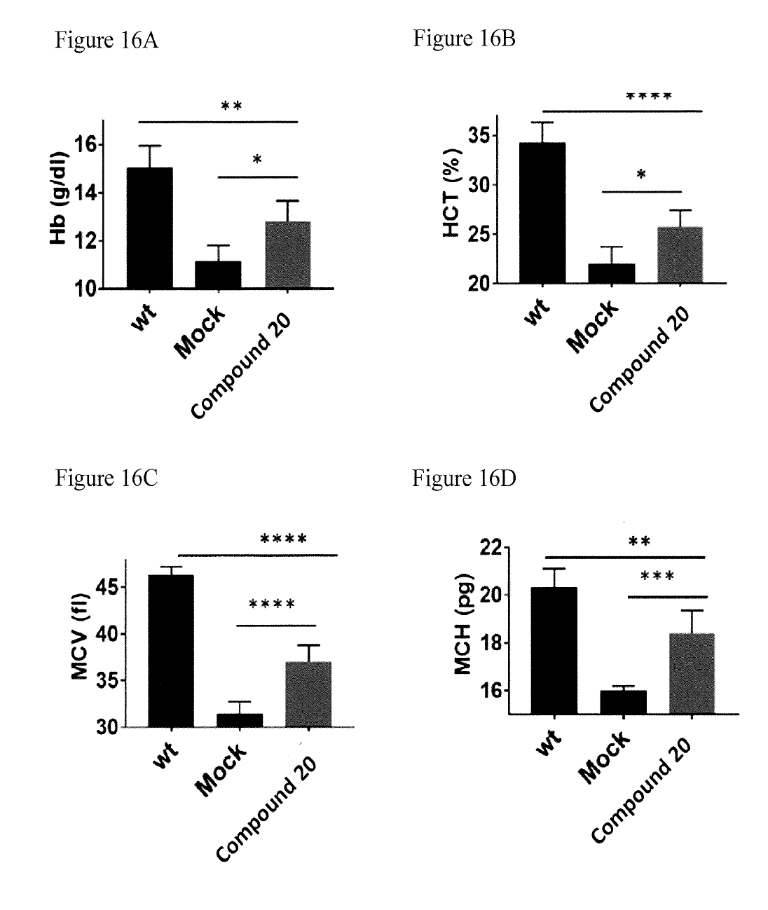

After sacrifice, serum iron and transferrin saturation were measured. The results were compared with data from WT C57BL6 mice to assess if the iron deficiency is improved and if the iron levels reach the same levels as WT mice. The serum iron and transferrin results after 7 weeks of treatment are summarized in FIGS. 15A and 15B. Additionally, FIGS. 16A-16D include data showing the compound 20 improves anemia in IRIDA model.

Collectively, the data suggest that treatment with compound 20 corrects serum iron deficiency, improves anemia, and shows no induction of inflammation. In addition, treatment is not toxic for the mice as no gross abnormality was observed in hematoxylin staining analysis.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound having a structure of Formula I or a pharmaceutically acceptable salt thereof: 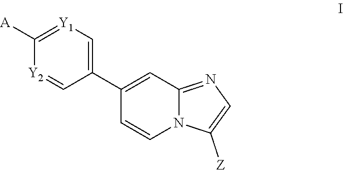
wherein
Y¹ and Y² are each independently CR¹ or N;
R¹ is, independently for each occurrence, H or alkyl;
A is optionally substituted alkoxy, cycloalkylalkoxy, heterocyclyl, heterocyclylalkoxy, or amino; and
Z is optionally substituted heteroaryl.

2. The compound of claim 1, wherein Y¹ is N and Y² is CR¹.

3. The compound of claim 1, wherein Y¹ and Y² are CR¹.

4. The compound of claim 3, wherein one R¹ is H and the other R¹ is alkyl.

5. The compound of claim 1, wherein Y¹ and Y² are N.

6. The compound of claim 1, wherein R¹ is H.

7. The compound of claim 1, wherein R¹ is alkyl, preferably lower alkyl.

8. The compound of claim 1, wherein
Z is 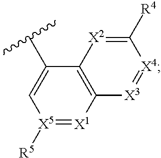
X¹, X², and X³ are each independently CR² or N, provided that at least one of X¹, X², and X³ is N;
X⁴ is CR³ or N;
X⁵ is C or N;
R², independently for each occurrence, is H, halo, or alkyl; and
R³, R⁴, and R⁵ are each independently H, halo, hydroxyl, cyano, optionally substituted alkyl, or optionally substituted alkoxy.

9. The compound of claim 8, wherein X¹ is N.

10. The compound of claim 8, wherein X² is N.

11. The compound of claim 8, wherein X³ is N.

12. The compound of claim 8, wherein X⁴ is N.

13. The compound of claim 8, wherein Z is

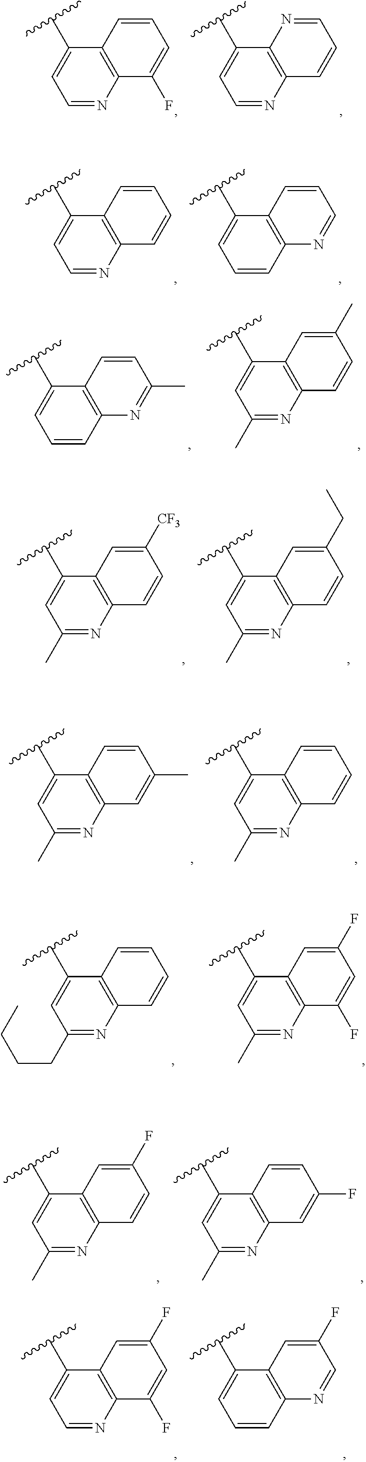

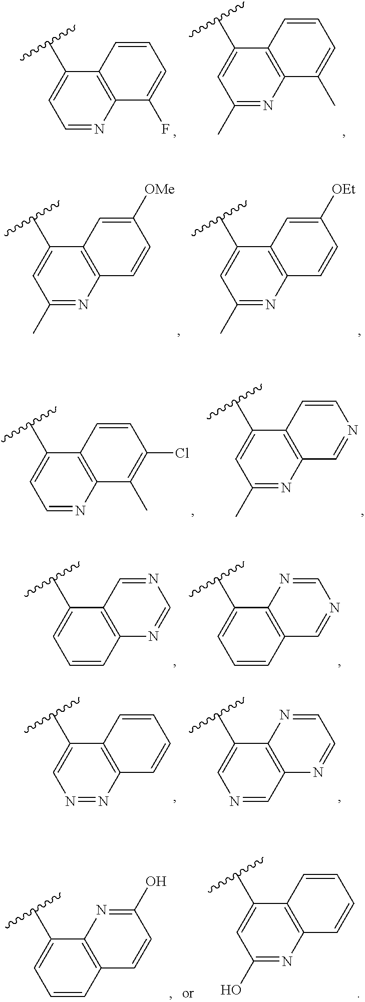

14. The compound of claim 1, wherein
Z is 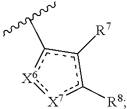
X⁶ and X⁷ are each independently CR⁵, S, or O provided that one of X⁶ and X⁷ is CR⁵;
each R⁵ is independently H, halo, cyano, or optionally substituted alkyl, acyl, carboxy, or carbonyl; and
R⁷ and R⁸ are each independently H, halo, cyano, optionally substituted alkyl, or amide; or
R⁷ and R⁸ combine to form an optionally substituted 6-membered heteroaryl ring.

15. The compound of claim 14, wherein X⁶ is S.

16. The compound of claim 14, wherein X⁷ is S.

17. The compound of claim 14, wherein X⁷ is O.

18. The compound of claim 14, wherein Z is,

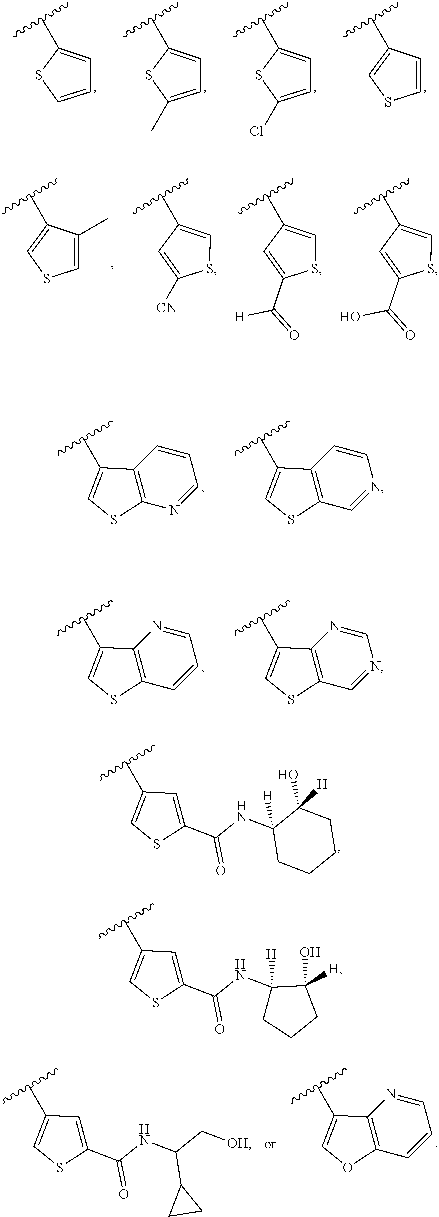

19. The compound of claim 1, wherein Z is

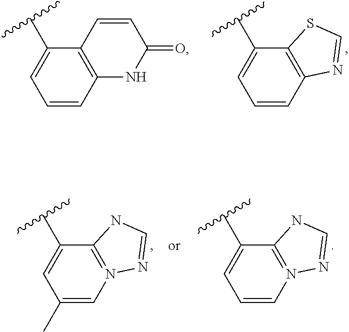

20. The compound of claim 1, wherein A is optionally substituted alkoxy, cycloalkylalkoxy, or heterocyclalkoxy.

21. The compound of claim 21, wherein A is,

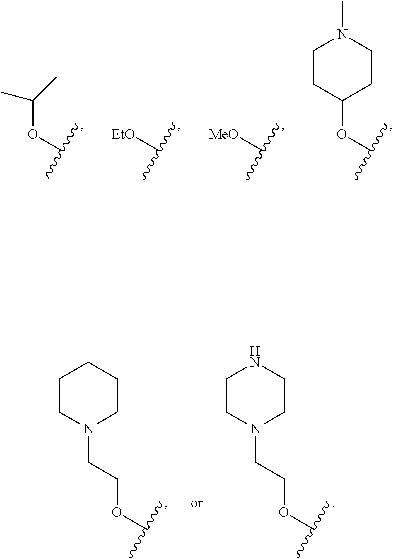

22. The compound of claim 1, wherein A is amino, alkylamino, heteroalkylamino, cycloalkylamino, cycloalkylalkylamino, heterocyclylamino, or heterocycloalkylamino.

23. The compound of claim 1, wherein A is an optionally substituted nitrogen-containing heterocyclyl.

24. The compound of claim 1, wherein
A is 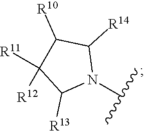
and
R¹⁰, R¹¹, R¹², R¹³, and R¹⁴ are each independently H, optionally substituted alkyl, or optionally substituted heterocyclyl, alkylaminoalkyl, or heterocycloalkyl; or
R¹⁰ and R¹² combine to form an optionally substituted 5-membered ring; or
R¹⁰ and R¹⁴ combine to form an optionally substituted 5-membered ring; or
R¹¹ and R¹² combine to form an optionally substituted 4-, 5-, or 6-membered ring.

25. The compound of claim 24, wherein the optionally substituted 4-, 5-, or 6-membered ring comprises a heteroatom.

26. The compound of claim 25, wherein the heteroatom is N.

27. The compound of claim 24, wherein A is

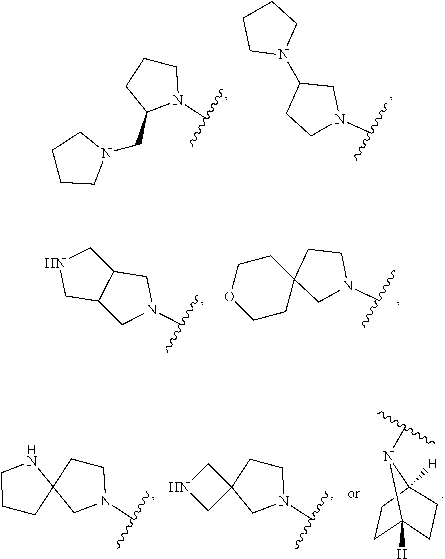

28. The compound of claim 1, wherein
A is 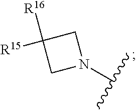
and
R¹⁵ and R¹⁶ are each independently H, halo, cyano, or alkyl; or
R¹⁵ and R¹⁶ combine to form an optionally substituted 4-, 5-, or 6-membered ring

29. The compound of claim 28, wherein A is

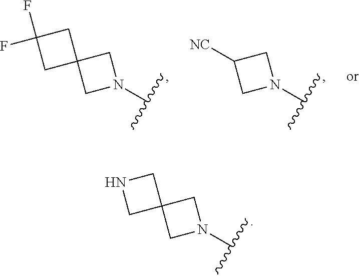 [SMILES: *N1CC2(C1)CC(F)(F)C2 ; *N1CC(C#N)C1 ; *N1CC2(CNC2)C1]

30. The compound of claim 1, wherein A is

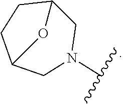 [SMILES: *N1CC2CCC(C1)O2]

31. The compound of claim 1, wherein
A is 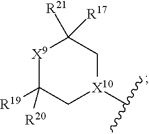
X⁹ is CHR¹⁸, NR¹⁸, or O;
X¹⁰ is CH or N;
R¹⁸ is H, optionally substituted alkyl, aryl, heteroaryl, cycloalkyl, heterocyclyl, cycloalkylalkyl, heterocycloalkyl, alkoxyalkyl, aralkyl, heteroaralkyl, —C(O)-alkyl, or sulfone; and
R²¹, R¹⁷, R¹⁸, and R¹⁹ are each independently H, halo, cyano, or alkyl; or
R²¹ and R¹⁸ combine to form an optionally substituted 4-, 5-, or 6-membered ring; or
R²¹ and R¹⁷ combine to form a carbonyl.

32. The compound of claim 31, wherein X⁹ is N.

33. The compound of claim 31, wherein X¹⁰ is N.

34. The compound of claim 31, wherein X⁹ is O and R¹⁸ is absent.

35. The compound of claim 31, wherein A is

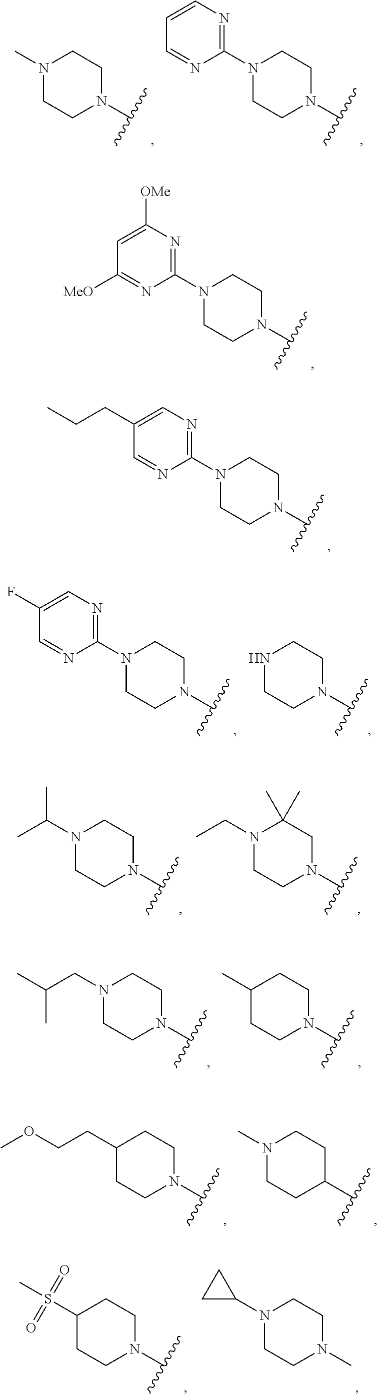

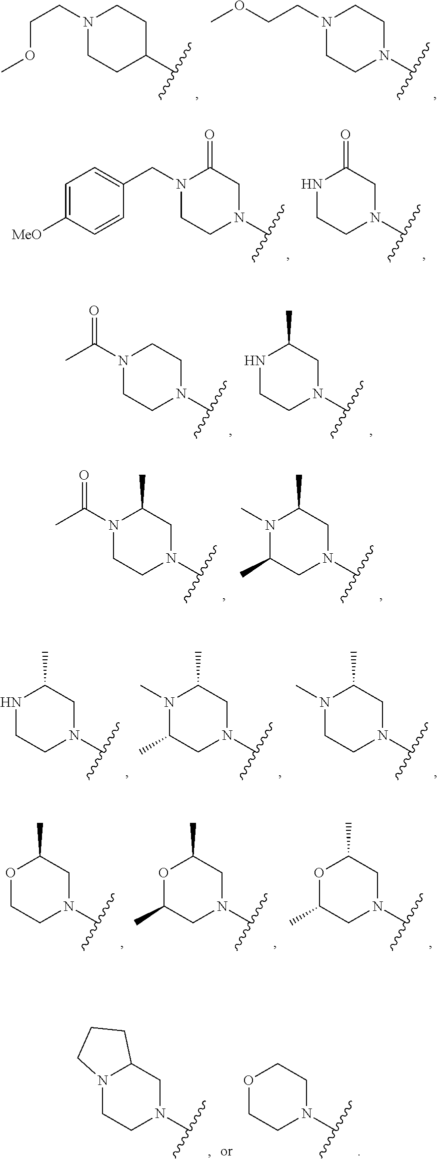

36. A compound selected from Table 1 or a pharmaceutically acceptable salt thereof.

37. The compound of claim 36, wherein the compound has the structure:

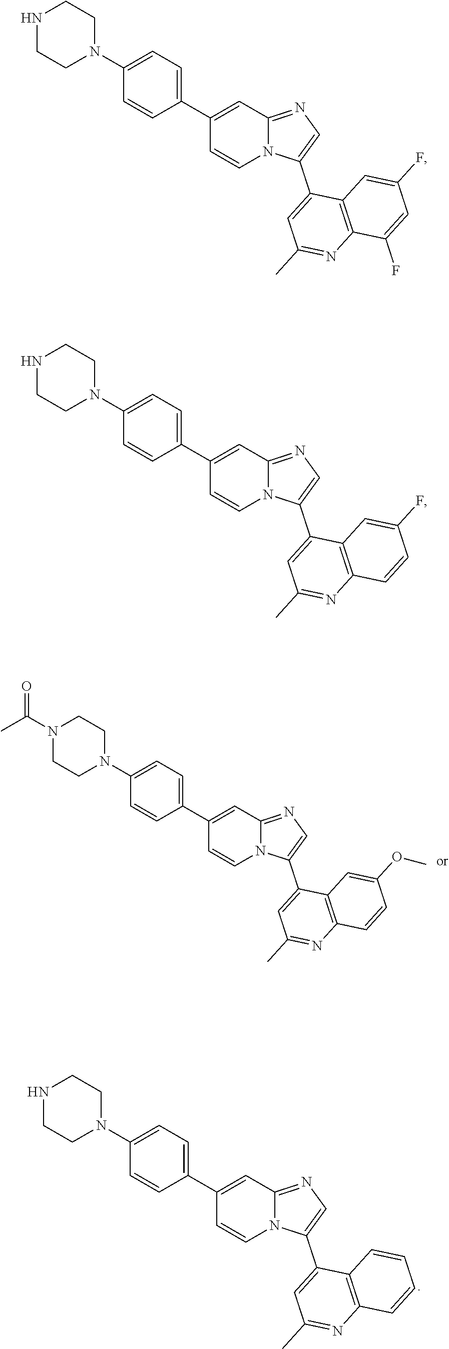 [SMILES: Cc1cc(-c2cnc3cc(-c4ccc(N5CCNCC5)cc4)ccn23)c2cc(F)cc(F)c2n1 ; Cc1cc(-c2cnc3cc(-c4ccc(N5CCNCC5)cc4)ccn23)c2cc(F)ccc2n1 ; COc1ccc2nc(C)cc(-c3cnc4cc(-c5ccc(N6CCN(C(C)=O)CC6)cc5)ccn34)c2c1 ; Cc1cc(-c2cnc3cc(-c4ccc(N5CCNCC5)cc4)ccn23)c2ccccc2n1]

38. A pharmaceutical composition comprising a compound of claim 1 and a pharmaceutically acceptable carrier.

39. A method of treating or preventing a disease or condition, comprising administering to a subject a composition of claim 38.

40. The method of claim 39, wherein the disease is cancer.

41. The method of claim 40, wherein the cancer is colorectal cancer, juvenile polyposis syndrome, sporadic colorectal cancer, leukemia, acute myeloid leukemia, acute megakaryoblastic leukemia (AMKL), non-Down syndrome AMKL, Down syndrome AMKL, chronic myelogenous leukemia, lung cancer, non-small cell lung cancer (NSCLC), pancreatic cancer, ovarian cancer, serous ovarian cancer, epithelial ovarian cancer, osteosarcomas, prostate cancer, bone cancer, renal cell cancer, breast cancer, melanoma, or head and neck squamous cell carcinoma (HNSCC).

42. The method of claim 40, wherein the cancer is a cancer of the central nervous system.

43. The method of claim 41, wherein the cancer is a glioma, astrocytic glioma, diffuse intrinsic pontine glioma (DIPG), high grade glioma (HGG), germ cell tumor, glioblastoma multiform (GBM), oligodendroglioma, pituitary tumor, or ependymoma.

44. The method of claim 39, wherein the disease is anemia, iron-refractory iron-deficient anemia (IRIDA), iron deficiency anemia, anemia of chronic disease, heterotopic ossification, nonhereditary myositis ossificans, myositis ossificans traumatica, myositis ossificans circumscripta, fibrodysplasia ossificans progressiva (FOP), inflammation, pathologic bone function, ectopic or maladaptive bone formation, a skin disease, hypertension, ventricular hypertrophy, atherosclerosis, spinal cord injury and neuropathy, heart disease, heart damage, liver damage, or liver disease.

45. A method of modulating the BMP signaling pathway, comprising contacting a cell with a composition of claim 38.

46. A method of inhibiting proliferation of a cancer cell, comprising contacting a cancer cell with a composition of claim 38.

47. A method for propagating, engrafting, or differentiating a progenitor cell, comprising contacting the cell with a composition of claim 38 in an amount effective to propagate, engraft, or differentiate the progenitor cell.
